(19) United States
(12) Patent Application Publication
(10) Pub. No.: US 2021/0093539 A1
(43) Pub. Date: Apr. 1, 2021

(54) TOPICAL COSMETIC

(71) Applicant: Concept Matrix Solutions, Woodland Hills, CA (US)

(72) Inventors: Tony LaRosa, Woodland Hills, CA (US); Robert Davidson, Woodland Hills, CA (US); David Reid, Woodland Hills, CA (US)

(21) Appl. No.: 16/946,999

(22) Filed: Jul. 14, 2020

Related U.S. Application Data
(60) Provisional application No. 62/907,780, filed on Sep. 30, 2019.
(60) Provisional application No. 62/977,854, filed on Feb. 18, 2020.

(51) Int. Cl. A61K 8/67; A61Q 19/00; A61K 8/34; A61K 8/37; A61K 8/31; A61K 8/24; A61K 8/42; A61K 8/81; A61K 8/891; A61K 8/41; A61K 8/73; A61K 8/63; A61K 8/44; A61K 8/9789; A61K 8/9794; A61K 8/49; A61K 8/96; A61Q 19/08; A61Q 19/02

(52) U.S. Cl. CPC A61K 8/678; A61K 2800/28; A61K 8/345; A61K 8/375; A61K 8/31; A61K 8/34; A61K 8/24; A61K 8/42; A61K 8/8147; A61K 8/891; A61K 8/416; A61K 8/37; A61K 8/735; A61K 8/63; A61K 8/41; A61K 8/73; A61K 8/44; A61K 8/9789; A61K 8/9794; A61K 8/49; A61K 8/965; A61Q 19/08; A61K 8/675; A61Q 19/02; A61K 2800/30; A61K 2800/48; A61Q 19/007

(57) ABSTRACT

Provided herein is a topical cosmetic composition that includes an external cosmetic agent, one or more pharmaceutically acceptable excipients, and at least one of a cannabinoid, terpene, and flavonoid. Also provided is a method that includes topically administering to a skin surface of a subject (e.g., human) the topical cosmetic composition.

### RELATED U.S. APPLICATION DATA

This application claims priority to provisional patent application number 62/907,780 filed on Sep. 30, 2019, and provisional patent application no. 62/977,854 filed Feb. 18, 2020, the contents of which are incorporated by reference herein in their entirety.

### SUMMARY OF THE INVENTION

The present invention provides for a topical cosmetic composition that includes an external cosmetic agent; solvent; emulsifier; thickening agent; emollient; and at least one of a cannabinoid, terpene, and flavonoid.

The present invention also provides for a method that includes topically administering to a skin surface of a subject (e.g., human adolescent or adult) the topical composition described herein.

The present invention also provides for a method that includes topically administering to a skin surface of a subject (e.g., human adolescent or adult) the topical composition described herein to treat or prevent at least one of: (i) wrinkles, (ii) fine lines, (iii) blemishes, (iv) skin discoloration, (v) dry skin, (vi) skin irritation, (vii) saggy skin, (viii) inelastic skin, (ix) enlarged pores, (x) acne scars, (xi) inflammation, (xii) crow's feet, (xiii) laugh lines, (xiv) drooping eyelids, (xv) crepey skin, (xvi) frown lines, (xvii) dull skin tone, (xviii) dark circles under the eyes, (xix) lackluster skin, (xx) itchy skin, (xxi) hyperpigmentation, (xxii) uneven skin tone, and (xxiii) collagen loss.

The present invention also provides for a method that includes topically administering to a skin surface of a subject (e.g., human adolescent or adult) the topical composition described herein to: help prevent or reduce signs of aging, cleanse the skin, remove excess oil, reduce the size and redness of acne scars, renew skin, normalize skin, exfoliate the skin, provide smooth & soft skin texture, invigorate skin by lifting away dead skin cells, clear skin, reduce appearance of pores, help balance skin's pH level, provide clearer complexion, and/or provide softer & smoother skin.

### DETAILED DESCRIPTION OF THE INVENTION

Compositions of the present invention may be beneficial in preventing, treating, managing, and/or ameliorating a variety of skin conditions due to aging. Specifically, the compositions may be beneficial in preventing, treating, managing, and/or ameliorating at least one of: (i) wrinkles, (ii) fine lines, (iii) blemishes, (iv) skin discoloration, (v) dry skin, (vi) skin irritation, (vii) saggy skin, (viii) inelastic skin, (ix) enlarged pores, (x) acne scars, (xi) inflammation, (xii) crow's feet, (xiii) laugh lines, (xiv) drooping eyelids, (xv) crepey skin, (xvi) frown lines, (xvii) dull skin tone, (xviii) dark circles under the eyes, (xix) lackluster skin, (xx) itchy skin, (xxi) hyperpigmentation, (xxii) uneven skin tone, and (xxiii) collagen loss. More specifically, the compositions may be beneficial to prevent or reduce signs of aging, cleanse the skin, remove excess oil, reduce the size and redness of acne scars, renew skin, normalize skin, exfoliate the skin, provide smooth & soft skin texture, invigorate skin by lifting away dead skin cells, clear skin, reduce appearance of pores, help balance skin's pH level, provide clearer complexion, and/or provide softer & smoother skin.

In another aspect, the invention relates to a method of preventing, treating or ameliorating signs of aging skin. The method includes administering to a person suffering from at least one of said conditions (or is at risk thereof), an effective amount of one or more compositions as described herein.

The compositions of the present invention include at least one of a cannabinoid, terpene, and flavonoid. Without wishing to be limited to any particular theory, it is currently believed that the cannabinoid, terpene, and/or flavonoid provides stability to the composition, which serves to prevent phase separation of an aqueous and a lipid phase in the composition at elevated temperatures (e.g. temperatures of more than about 25° C.), which might promote improved or prolonged contact to the skin, resulting in the observed increased retention times of the active(s) in the dermis and epidermis.

Across multiple topical dosage forms (e.g., creams, gels, lotions, ointments, foams, etc.) the cannabinoid, terpene, and/or flavonoid can be present in an amount, such that it exhibits activity as an active ingredient for the intended purpose (e.g., approving the appearance of skin). In doing so, the cannabinoid, terpene, and/or flavonoid may further have a synergistic effect with the active ingredient(s) present therein. In other aspects, the cannabinoid, terpene, and/or flavonoid can be present in sub-therapeutic amounts.

It is currently believed that the topical use of the cannabinoid, terpene, and/or flavonoid provides additional benefits, which include: smoothing skin, strengthening underlying epidermal tissue, removing dead skin cells, balancing oil production, and helping the skin retain moisture levels. It is further believed that the cannabinoid, terpene, and/or flavonoid helps cleanse and moisturize the skin. Healthy skin is just like any other organ in your body: It continuously needs oxygen and nutrients to be brought to the cells, and toxins need to be washed away. It is further believed that the cannabinoid, terpene, and/or flavonoid contributes to the optimal skin health while leaving skin feeling and looking youthful.

It is also currently believed that some consumers have a preference to use a topical skin product that is environmentally friendly to produce and includes fewer toxic chemicals that are otherwise put into the environment when creating topical skin products. Inclusion of substances, such as cannabinoid, terpene, and flavonoid (which are natural products), are viable options for such consumers.

### Definitions:

The term “topical cosmetic composition” refers to a topical composition containing an external topical cosmetic agent, various inactive ingredients or excipients, and at least one of a terpene and cannabinoid. The composition is suitable for the topical treatment of aging skin.

The term “cosmetic” refers to a topical treatment intended to restore or improve a person's appearance, such as preventing or reducing the signs of aging skin. Representative signs of aging skin includes, for example wrinkles, fine lines, blemishes, skin discoloration, dry skin, skin irritation, saggy skin, inelastic skin, enlarged pores, acne scars, inflammation, crow's feet, laugh lines, drooping eyelids, crepey skin, frown lines, dull skin tone, dark circles under the eyes, lackluster skin, itchy skin, hyperpigmentation, uneven skin tone, and collagen loss).

The term “external cosmetic agent” or “cosmetic agent” refers to any chemical and/or biological agent (i.e. an antimicrobial peptide) that when topically administered at the intended site on the skin, effectively treats and/or leads to a visible reduction of signs associated with aging skin. Representative cosmetic agents include, for example retinol (vitamin A), green tea extract, acai oil, alpha-lipoic acid, Coenzyme Q10, caffeine, glycolic acid, lactic acid, citric acid, tartaric acid, salicylic acid, L-ascorbic acid, hyaluronic acid, grape seed extract, niacinamide, hydroquinone, jojoba oil, kojic acid, malic acid, resveratrol, vitamin E (tocopherol, tocopheryl acetate), vitamin K, ceramides, raspberry seed oil, plantain extract, collagen, Centella Asiatica, acetyl hexapeptide-3, acetyl hexapeptide-8, beta glucan, carrot root extract, edelweiss extract, ginger root extract, Ginkgo biloba leaf extract, Goji berry extract, Helichrysum extract, polygonum aviculare extract, Vaccinium Vitis Idaea (lingonberry) fruit extract, orange callus culture extract, ferulic acid, tomato extract, teprenone, watermelon extract, turmeric, curcumin, dimethylaminoethanol, elastin, vitamin B5, magnesium oil, colloidal oatmeal, cocoa butter, squalane oil, argan oil, cupuacu butter, avocado oil, Hawaiian kukui nut oil, white tea extract, goat milk, glycerin, allantoin, lanolin and dimethicone.

The term “solvent” refers to a substance, typically a liquid at ambient conditions, capable of dissolving another substance (a solute), resulting in a solution. When one substance is dissolved into another, a solution is formed. This is opposed to the situation when the compounds are insoluble like sand in water. In solution, all of the ingredients are uniformly distributed at a molecular level and no residue remains. The mixing is referred to as miscibility, whereas the ability to dissolve one compound into another is known as solubility. However, in addition to mixing, both substances in the solution interact with each other. When something is dissolved, molecules of the solvent arrange themselves around molecules of the solute. Heat is involved and entropy is increased making the solution more thermodynamically stable than the solute alone. This arrangement is mediated by the respective chemical properties of the solvent and solute, such as hydrogen bonding, dipole moment and polarizability.

The term “emulsifier” refers to a substance capable of forming or promoting an emulsion. An emulsion is a mixture of two or more liquids that are normally immiscible (nonmixable or unblendable). Emulsions are part of a more general class of two-phase used systems of matter called colloids. Although the terms colloid and emulsion are sometimes interchangeable, emulsion should be used when both the dispersed and the continuous phase are liquids. In an emulsion, one liquid (the dispersed phase) is dispersed in the other (the continuous phase). Examples of emulsions include vinaigrettes, milk, mayonnaise, and some cutting fluids for metal working. The photo-sensitive side of photographic film is an example of a colloid.

The term “thickening agent” or “viscosity-increasing agent” refers to a substance which can increase the thickness or viscosity of a liquid without substantially changing its other properties. Some thickening agents are gelling agents (gellants), forming a gel, dissolving in the liquid phase as a colloid mixture that forms a weakly cohesive internal structure. Others act as mechanical thixotropic additives with discrete particles adhering or interlocking to resist strain. Typical gelling agents include natural gums, starches, pectins, agar-agar and gelatin. Often they are based on polysaccharides or proteins. Examples include: Alginic acid, sodium alginate, potassium alginate, ammonium alginate, calcium alginate—polysaccharides from brown algae; Agar (polysaccharide obtained from red algae); Carrageenan (polysaccharide obtained from red seaweeds); Locust bean gum (natural gum polysaccharide from the seeds of the carob tree); Pectin (polysaccharide obtained from apple or citrus-fruit); and Gelatin (made by partial hydrolysis of animal collagen). The thickening agent or viscosity-increasing agent also includes cellulose thickeners, such as e.g., hydroxypropyl cellulose, hydroxyethyl cellulose, hydroxypropyl cellulose, hydroxypropyl methyl cellulose, methyl cellulose, carbopols, polyacrylic acid, and polyvinyl alcohol.

The term “emollient” refers to lubricating ingredients (i.e., fats, phospholipids and sterols) that soften and smooth skin while helping it to retain moisture. Emollients are typically nonpolar and can come from natural or synthetic sources in the form of plant oils, mineral oils, shea butter, cocoa butter, petrolatum, cholesterol, silicones or animal oils (including emu, mink and lanolin).

The term “cannabinoid” refers to a class of diverse chemical compounds that act on cannabinoid receptors on cells that repress neurotransmitter release in the brain. These receptor proteins include the endocannabinoids (produced naturally in the body by humans and animals), the phytocannabinoids (found in Cannabis and some other plants), and synthetic cannabinoids (manufactured chemically). The most notable cannabinoid is the phytocannabinoid Δ9-tetrahydrocannabinol (THC), the primary psychoactive compound of Cannabis. Cannabidiol (CBD) is another major constituent of the plant, representing up to 40% in extracts of the plant resin. There are at least 85 different cannabinoids isolated from Cannabis, exhibiting varied effects. The cannabinoid can be synthetically prepared, or alternatively, can be obtained naturally (e.g., from plant matter). Either way, the cannabinoid can have the requisite purity (e.g., at least 95 wt. % pure, at least 98 wt. % pure, at least 99 wt. % pure, or at least 99.5 wt. % pure).

Cannabinoids isolated from Cannabis

| | |
|---|---|
| 1. | Cannabigerol ((E)-CBG-C5) |
| 2. | Cannabigerol monomethyl ether ((E)-CBGM-C5 A) |
| 3. | Cannabinerolic acid A ((Z)-CBGA-C5 A) |
| 4. | Cannabigerovarin ((E)-CBGV-C3) |
| 5. | Cannabigerolic acid A ((E)-CBGA-C5 A) |
| 6. | Cannabigerolic acid A monomethyl ether ((E)-CBGAM-C5 A) |
| 7. | Cannabigerovarinic acid A ((E)-CBGVA-C3 A) |
| 8. | (±)-Cannabichromene (CBC-C5) |
| 9. | (+)-Cannabichromenic acid ACBCA-C5 A |
| 10. | (+)-Cannabivarichromene or (+)-Cannabichromevarin (CBCV-C3) |
| 11. | (+)-Cannabichromevarinic acid A (CBCVA-C3 A) |
| 12. | (−)-Cannabidiol (CBD-C5) |
| 13. | Cannabidiol momomethyl ether (CBDM-C5) |
| 14. | Cannabidiol-C4 (CBD-C4) |
| 15. | (−)-Cannabidivarin CBDV-C3 |
| 16. | Cannabidiorcol (CBD-C1) |
| 17. | Cannabidiolic acid (CBDA-C5) |
| 18. | Cannabidivarinic acid (CBDVA-C3) |
| 19. | Cannabinodiol (CBND-C5) |
| 20. | Cannabinodivarin (CBND-C3) |
| 21 | Δ9-Tetrahydrocannabinol (Δ9-THC-C5) |
| 22. | Δ9-Tetrahydrocannabinol-C4 (Δ9-THC-C4) |
| 23. | Δ9-Tetrahydrocannabivarin (Δ9-THCV-C3) |
| 24. | Δ9-Tetrahydrocannabiorcol (Δ9-THCO-C1) |
| 25. | Δ9-Tetrahydro- cannabinolic acid A (Δ9-THCA-C5 A) |
| 26. | Δ9-Tetrahydro- cannabinolic acid B (Δ9-THCA-C5 B) |
| 27. | Δ9-Tetrahydro- cannabinolic acid-C4A and/or B (Δ9-THCA-C4A |
| | and/or B) |
| 28. | Δ9-Tetrahydro- cannabivarinic acid A (Δ9-THCVA-C3A) |
| 29. | Δ9-Tetrahydro- cannabiorcolic acid A and/or B (Δ9-THCOA-C1A |
| | and/or B) |
| 30. | (−)-Δ8-trans-(6aR,10aR)-Δ8-Tetrahydrocannabinol (Δ8-THC-C5) |
| 31. | (−)-Δ8-trans-(6aR,10aR)- Tetrahydrocannabinolic acid A (Δ8- |
| | THCA-C5 A) |
| 32. | (−)-(6aS,10aR)-Δ9-Tetrahydrocannabinol ((−)-cis-Δ9-THC-C5) |
| 33. | Cannabinol (CBN-C5) |
| 34. | Cannabinol-C4 (CBN-C4) |
| 35. | Cannabivarin (CBN-C3) |
| 36. | Cannabinol-C2 (CBN-C2) |
| 37. | Cannabiorcol (CBN-C1) |
| 38. | Cannabinolic acid A (CBNA-C5 A) |
| 39. | Cannabinol methyl ether (CBNM-C5) |
| 40. | (−)-(9R,10R)-trans- Cannabitriol ((−)-trans-CBT-C5) |
| 41. | (+)-(9S,10S)-Cannabitriol ((+)-trans-CBT-C5) |
| 42. | (±)-(9R,10S/9S,10R)- Cannabitriol ((±)-cis-CBT-C5) |
| 43. | (−)-(9R,10R)-trans- 10-O-Ethyl-cannabitriol ((−)-trans-CBT-OEt-C5) |
| 44. | (±)-(9R,10R/9S,10S)- Cannabitriol-C3 ((±)-trans-CBT-C3) |
| 45. | 8,9-Dihydroxy-Δ6a(10a)- tetrahydrocannabinol (8,9-Di-OH-CBT-C5) |
| 46. | Cannabidiolic acid A cannabitriol ester (CBDA-C5 9-OH-CBT-C5 |
| | ester) |
| 47. | (−)-(6aR,9S,10S,10aR)- 9,10-Dihydroxy- hexahydrocannabinol, |
| | Cannabiripsol (Cannabiripsol-C5) |
| 48. | (−)-6a,7,10a-Trihydroxy-Δ9-tetrahydrocannabinol ((−)-Cannabitetrol) |
| 49. | 10-Oxo-Δ6a(10a)- tetrahydrocannabinol (OTHC) |
| 50. | (5aS,6S,9R,9aR)- Cannabielsoin (CBE-C5) |
| 51. | (5aS,6S,9R,9aR)- C3-Cannabielsoin (CBE-C3) |
| 52. | (5aS,6S,9R,9aR)- Cannabielsoic acid A (CBEA-C5 A) |
| 53. | (5aS,6S,9R,9aR)- Cannabielsoic acid B (CBEA-C5 B) |
| 54. | (5aS,6S,9R,9aR)- C3-Cannabielsoic acid B (CBEA-C3 B) |
| 55. | Cannabiglendol-C3 (OH-iso-HHCV-C3) |
| 56. | Dehydrocannabifuran (DCBF-C5) |
| 57. | Cannabifuran (CBF-C5) |
| 58. | (−)-Δ7-trans-(1R,3R,6R)-Isotetrahydrocannabinol |
| 59. | (±)-A7-1,2-cis- (1R,3R,6S/1S,3S,6R)- Isotetrahydro- cannabivarin |
| 60. | (−)-Δ7-trans-(1R,3R,6R)- Isotetrahydrocannabivarin |
| 61. | (±)-(1aS,3aR,8bR,8cR)- Cannabicyclol (CBL-C5) |
| 62. | (±)-(1aS,3aR,8bR,8cR)- Cannabicyclolic acid A (CBLA-C5 A) |
| 63. | (±)-(1aS,3aR,8bR,8cR)- Cannabicyclovarin (CBLV-C3) |
| 64. | Cannabicitran (CBT-C5) |
| 65. | Cannabichromanone (CBCN-C5) |
| 66. | Cannabichromanone-C3 (CBCN-C3) |
| 67. | Cannabicoumaronone (CBCON-C5) |
| 68. | Cannabielsoin acid A (CBEA-A) |
| 69. | 10-Ethoxy-9-hydroxy-delta-6a-tetrahydrocannabinol |
| 70. | Cannabitriolvarin (CBTV) |
| 71. | Delta-9-tetrahydrocannabiorcolic acid (THCA-C1) |
| 72. | Delta-7-cis-iso-tetrahydrocanna |
| 73. | Cannabichromanon (CBCF) |

```
Structure of common cannaboinoids
  CBD
  CBDA
  CBDVA-OMe
  THC
  THCA
  THCV
  CBG
  CBGA
  CBGV
  CBN
  CBQ
  CBC
  CBL
  CBGB
  CBGBA
  CVDBA
  CBDVA
  THCVA
```

Synthetically prepared cannabinoids, that are commercially available (e.g., Purisys™ of Athens, Ga.), are provided below.

| | | | |
|---|---|---|---|
| | | Alkyl Tail | |
| Common Name | Other Names | Length | CAS # |

TETRAHYDROCANNABIVARIN FAMILY

| | | | |
|---|---|---|---|
| Δ9-Tetrahydrocannabivarin | Δ9-THCV | C3 | 31262-37-0 |
| Δ8-Tetrahydrocannabivarin | Δ8-THCV | C3 | 31262-38-1 |
| Δ9-Tetrahydrocannabivarin | Δ9-THCV-NE | C3 | N/A |
| Naphtoylester | | | |
| Δ8-Tetrahydrocannabivarin | Δ8-THCV-NE | C3 | N/A |
| Naphtoylester | | | |
| Δ9-Tetrahydrocannabivarinic | Δ9-THCVA-A, | C3 | 39986-26-0 |
| Acid | Δ9-THC-VA-B | | |

| | | | |
|---|---|---|---|
| | | Alkyl Tail | |
| Common Name | Other Names | Length | CAS # |

CANNABIDIOLVARIN FAMILY

| | | | |
|---|---|---|---|
| (−)-Cannabidivarin | (−)-CBDV | C3 | 24274-48-4 |
| (+)-Cannabidivarin | (+)-CBDV | C3 | 1637328-94-9 |
| Cannabidivarinic Acid | CBDVA | C3 | 31932-13-S |
| Cannabidivarin Quinone | CBQV | C3 | N/A |

| | | | |
|---|---|---|---|
| | | Alkyl Tail | |
| Common Name | Other Names | Length | CAS # |

TETRAHYDROCANNABIBUTOL FAMILY

| | | | |
|---|---|---|---|
| Δ9-Tetrahydrocannabibutol | Δ9-THCB | C4 | 60008-00-6 |
| Δ8-Tetrahydrocannabibutol | Δ8-THCB | C4 | 51768-59-3 |
| Δ9-Tetrahydrocannabibutol | Δ9-THCB-NE | C4 | 60007-98-9 |
| Naphtoylester | | | |
| Δ8-Tetrahydrocannabibutol | Δ8-THCB-NE | C4 | N/A |
| Naphtoylester | | | |
| Δ9-Tetrahydrocannabibutolic | Δ9-THCBA-A, | C4 | 60007-98-9 |
| Acid | Δ9-THC-BA-B | | |

| | | | |
|---|---|---|---|
| | | Alkyl Tail | |
| Common Name | Other Names | Length | CAS # |

CANNABIDIBUTOL FAMILY

| | | | |
|---|---|---|---|
| (−)-Cannabidibutol | (−)-CBDB | C4 | 60113-11-3 |
| (+)-Cannabidibutol | (+)-CBDB | C4 | N/A |
| Cannabidibutolic Acid | CBDBA | C4 | N/A |

| | | | |
|---|---|---|---|
| | | Alkyl Tail | |
| Common Name | Other Names | Length | CAS # |

RARE CANNABINOIDS

| | | | |
|---|---|---|---|
| Cannabinol | CBN, USP | C5 | 521-35-7 |
| | Impurity | | |
| Cannabinolic Acid | CBNA | C5 | 2808-39-1 |
| Cannabigerol | CBG | C5 | 25654-31-3 |
| Cannabigerolic Acid | CBGA | C5 | 25555-57-1 |
| Cannabichromene | CBC | C5 | 20675-51-8 |
| Cannabichromenic Acid | CBCA | C5 | 185505-15-1 |
| Cannbicyclol | CBL | C5 | 21366-63-2 |
| Cannabicyclolic Acid | CBLA | C5 | 2283314-84-9 |
| Cannabivarin | CBNV | C3 | 33745-21-0 |
| Cannabivarinic Acid | CBNVA | C3 | 64846-02-2 |
| Cannbigerivarin | CBGV | C3 | 55824-11-8 |
| Cannabigerivarinic Acid | CBGVA | C3 | 64924-07-8 |
| Cannbichromevarin | CBCV | C3 | 57130-04-8 |
| Cannabichromevarinic Acid | CBCVA | C3 | 64898-02-8 |
| Cannabicyclolvarin | CBLV | C3 | 55870-47-8 |
| Cannabicyclolvarinic Acid | CBLVA | C3 | 2281847-63-8 |
| 3-Butylcannabinol | CBNB | C4 | 60007-99-0 |
| 3-Butylcannabinolic Acid | CBNBA | C4 | N/A |
| Cannabigerol Butyl | CBGB | C4 | N/A |
| Cannabigerol Butyric Acid | CBGBA | C4 | N/A |
| Cannabichromene Butyl | CBCB | C4 | N/A |
| Cannabichromene Buytric | CBCBA | C4 | N/A |
| Acid | | | |
| Cannabicyclol Butyl | CBLB | C4 | N/A |
| Cannabicyclol Butyric Acid | CBLBA | C4 | N/A |

The term “terpene” refers to a hydrocarbon or derivative thereof, found as a natural product and biosynthesized by oligomerization of isoprene units. A terpene can be acyclic, monocyclic, bicyclic, or multicyclic. Examples include, e.g., sesquiterpenes (e.g., (−)-β-caryophyllene, humulene, vetivazulene, guaiazulene, longifolene, copaene, and patchoulol), monoterpenes (e.g., limonene and pulegone), monoterpenoids (e.g., carvone), diterpenes (e.g., taxadiene), and triterpenes (e.g., squalene, betulin, betulinic acid, lupane, lupeol, betulin-3-caffeate, allobetulin, and cholesterol). The terpene can be synthetically prepared, or alternatively, can be obtained naturally (e.g., from plant matter). Either way, the terpene can have the requisite purity (e.g., at least 95 wt. % pure, at least 98 wt. % pure, at least 99 wt. % pure, or at least 99.5 wt. % pure).

| Terpene | Plant | Genus | Species |
|---|---|---|---|
| Myrcene | Myrtles | Myrtus | communis; nivellei; |
| | | | phyllireaefolia |
| | Cannabis | Cannabis | sativa; ruderalis; indica |
| Linalool | Mint | Mentha | spicata; arvensis; |
| | | | canadensis |
| | Lavender | Lavandula(subgenus: | spica; angustifolia; |
| | | Fabricia; Sabaudia) | latifolia; lanata; |
| | | | dentata; stoechas; |
| | | | pedunculata; viridis |
| Terpineol | Orange peel | Citrus | reticulata |
| | Junipers | Juniperus | communis; chinensis; |
| | | | conferta; rigida |
| Camphene | Chrysanthemum | Chrysanthemum | indicum |
| | Ginger | Zingiber | officinale |
| Bisabolol | Chamomile | Matricaria (or | chamomilla (or nobile) |
| | | Chamaemelum) | |
| | Figwort | Myoporum | crassifolium |
| Nerolidol | Cannabis | Cannabis | sativa; ruderalis; indica |
| Limonene | Citrus Lemon | Citrus | limon |
| Humulene | Hops | Humulus | lupulus; japonicus; |
| | | | yunnanensis |
| Terpinolene | Cannabis | Cannabis | sativa; ruderalis; indica |
| Carene | Rosemary | Salvia | rosmarinus; jordanii |
| | Cedar | Cedrus | atlantica; brevifolia; |
| | | | deodara; libani |
| Eucalyptol | Eucalyptus | Eucalyptus | obliqua |
| | Cannabis | Cannabis | sativa; ruderalis; indica |
| | Camphor laurel | Cinnamomum | camphora |
| | Bay leaves | Laurus | nobilis |
| | Wormwood | Artemisia | vulgaris |
| Ocimene | Hops | Humulus | lupulus; japonicus; |
| | | | yunnanensis |
| | Kumquats | Citrus | japonica |
| | Mango | Mangifera | indica |
| | Basil | Ocimum | basilicum |
| | bergamot orange | Citrus | x aurantium |
| Carophyllene | Peppercorn | Piper | nigrum |
| | Cloves | Syzgium | aromaticum |
| | Cannabis | Cannabis | sativa; ruderalis; indica |
| | Rosemary | Salvia | rosmarinus; jordanii |
| | Hops | Humulus | lupulus; japonicus; |
| | | | yunnanensis |
| Valencene | Nootka cypress | Callitropsis | nootkatensis |
| Geraniol | Roses | Rosa (subgenus: | persica; minutifolia; |
| | | Banksianae, Bracteatae, | stellata |
| | | Caninae, Carolinae, | |
| | | Chinensis, Gallicanae, | |
| | | Gymnocarpae, | |
| | | Laevigatae, | |
| | | Pimpinellifoliae, | |
| | | Synstylae) | |
| | Wine grapes | Vitis | vinifera |
| Borneol | Borneo camphor | Dryobalanops | aromatica |
| | Ngai camphor; | Blumea | balsamifera |
| | sambong | | |
| Pulegone | Catnip | Nepeta | cataria |
| | Peppermint | Mentha | piperita |
| | Pennyroyal | Hedeoma | pulegioides |
| Guaiazulene | Chamomile | Matricaria (or | chamomilla (or nobile) |
| | | Chamaemelum) | |
| | Guaiacum tree | Guaiacum | sanctum, angustifolium, |
| | | | coulteri, officinale |
| Lupeol | Lupine seed | Lupinus | luteus |
| Lupane | Lupine seed | Lupinus | luteus |
| Betulin | Brich tree | Betula (Subgenus: | alleghaniensis, cordifolia, |
| Betulinic acid | | Betulenta, Betulaster, | glandulosa, lenta, |
| Lupeol | | Neurobetula, | michauxii, minor, nana, |
| | | Chamaebetula) | neoalaskana, nigra, |
| | | | occidentalis, papyrifera, |
| | | | populifolia, pumila, uber |
| Squalene | Amaranth seed | Amaranthus (subgenus: | acanthochiton, |
| | | Acnida; Albersia) | acutilobus, albus, |
| | | | anderssonii, californicus |
| | Wheat germ | Triticum | aestivum |
| | Olive | Olea | europaea |
| Carvone | Caraway seed | Carum | carvi |
| | Spearmint | Mentha | spicata |
| | Dill | Anethum | graveolens |
| Patchoulol | Patchouli | Pogostemon | cablin |
| Copaene | Copaiba tree | Copaifera | langsdorfii |
| Longifolene | Pine | Pinus | longifolia |
| Pinene | Pine | Pinus (subgenus: | densata, densiflora, |
| | | Strobus; Pinus) | pinea, sylvestris |
| Vetivazulene | Vetiver | Chrysopogon | zizanioides |
| Nerol | Lemon Grass | Cymbopogon | nardus; citratus; |
| | | | flexuosus; martinii; |
| | | | schoenanthus |

Synthetically prepared terpenes, which are commercially available (e.g., Purisys™ of Athens, Ga.), are provided below.

| Terpene | CAS# |
|---|---|
| Alpha-Pinene | 51634232009 |
| Beta-Pinene | 51634232109 |
| Beta-Myrcene | 51634232209 |
| Alpha-Terpinene | 51634232309 |
| Limonene | 51634232409 |
| Beta-Ocimene | 51634232509 |
| Terpinolene | 51634232609 |
| Linalool | 51634232709 |
| Fenchyl Alcohol | 51634232809 |
| Borneol Isomers | 51634232909 |
| Alpha-Terpineol | 51634233009 |
| Trans-caryophyllene | 51634233109 |
| Alpha-humulene | 51634233209 |
| Trans-nerolidol | 51634233309 |
| Guaiol | 51634233409 |
| Alpha-Bisabolol | 51634233509 |

The term “flavonoid” refers to ubiquitous plant natural products with various polyphenolic structures. Flavonoids can be extracted from fruits, vegetables, grains, bark, roots, stems, flowers, and teas or can be biosynthetically produced. The role of flavonoids in plants includes UV protection, aid in plant growth, defense against plaques, and provide the color and aroma of flowers.

Flavonoids can be divided into classes (e.g., anthocyanin, chalcone, flavone, flavonol, isoflavone, and flavonone) and subclasses depending on the carbon of the C ring on which the B ring is attached and the degree of unsaturation and oxidation of the C ring.

| | | | Examples of |
|---|---|---|---|
| Flavonoid classes | Subclasses | Natural sources | natural sources |
| Anthocyanins | Cyanidin, Malvidin, | Fruits, vegetables, | Cranberries, plums, |
| | Delphinidin, Peonidin | nuts, dried fruits, | cherries, sweet |
| | | medicinal plants | potatoes, black |
| | | | currants, red |
| | | | grapes, merlot |
| | | | grapes, raspberries, |
| | | | strawberries, |
| | | | blueberries, |
| | | | bilberries and |
| | | | blackberries |
| Chalcones | Phloretin, Arbutin, | Fruits, vegetables, | Tomatoes, pears, |
| | Phlioridzin | medicinal plants | strawberries, |
| | | | bearberries and |
| | | | certain wheat |
| | | | products |
| Flavonones | Hesperitin, Naringin, | Fruits (citrus), | Oranges, lemons, |
| | Naringenin, Eriodictyol, | medicinal plants | grapes, rosehips |
| | Hesperidin | | |
| Flavones | Apigenin, Tangeretin, | Fruits, medicinal | Celery, parsley, red |
| | Baicalein, Rpoifolin | plants | peppers, chamomile, |
| | | | mint, ginkgo biloba, |
| | | | broccoli, green |
| | | | pepper, thyme, |
| | | | dandelion, perilla, |
| | | | tea, carrot, |
| | | | rosemary, oregano, |
| | | | Cannabis sativa |
| Flavonols | Quercetin, Myricetin, | Fruits, vegetables, | Onion, kale, |
| | Rutin, Morin, | medicine plants | lettuce, tomatoes, |
| | Kaempferol | | apples, grapes, |
| | | | berries, tea, red |
| | | | wine, broccoli, |
| | | | potatoes, brussel |
| | | | sprouts, squash, |
| | | | cucumbers, lettuce, |
| | | | green beans, |
| | | | spinach, peaches, |
| | | | blackberries |
| Isoflavonoids | Genistin, Genistein, | Legumes, medicinal | Soybeans, lupin, |
| | Daidzein, Glycetein, | plants | fava beans, kudzu, |
| | Daidzin | | psoralea, red |
| | | | clover, alfalfa |
| | | | sprouts, peanuts, |
| | | | chickpeas |

| Flavonoid classes | Structure of flavonoid classes |
|---|---|
| Anthocyanins | Double bonds between positions 1 and 2, 3 and 4 of |
| | the C ring; Hydroxyl groups at positions 5 and 7 |
| | in the A ring and 3′, 4′ and/or 5′ of the B ring; |
| | Methylation or acylation at the hydroxyl groups |
| | on the A and B rings vary |
| Chalcones | Absence of ‘C ring’ of the basic flavonoid |
| | skeleton structure |
| Flavonones | C ring is saturated (contains no double bonds) |
| Flavones | Double bond between positions 2 and 3 and a ketone |
| | in position 4 of the C ring; Most have a hydroxyl |
| | group in position 5 or 7 of the A ring of the A |
| | ring or 3′ and 4′ of the B ring (varies according |
| | to the taxonomic classification of theparticular plant) |
| Flavonols | Double bond between positions 2 and 3, a ketone in |
| | position 4 and hydroxyl group in position 3 of the C |
| | ring; the ketone group the C ring may also be |
| | glycosylated; very diverse in methylation and |
| | hydroxylation patterns |
| Isoflavonoids | B ring is attached to the 3 position of the C ring and |
| | contains a hydroxyl group at the 4′ position; |
| | hydroxylation of the A ring varies |

Studies on flavonoids have revealed an increasing number of health benefits showing anti-oxidant, anti-inflammatory, anti-mutagenic, and anti-carcinogenic properties by inhibiting numerous pro-inflammatory and pro-oxidative enzymes (e.g., xanthine oxidase (XO), cyclo-oxygenase (COS), lipoxygenase, phosphoinositide 3-kinase, and acetylcholinesterase). This may have benefits towards numerous diseases and medical conditions (e.g., pain, cancer, arthersclerosis, Alzheimer's disease). There is a growing interest in the medicinal properties of Cannabis (Cannabis sativa, Cannabis indica, Cannabis ruderalis). Studies have shown that Cannaflavin A and Cannflavin B, prenylated flavones, have anti-inflammatory properties greater than aspirin. Cannflavin A and B can be isolated from Cannabis sativa and biosynthesized.

Synthetically prepared flavonoids, which are commercially available (e.g., Cannflavin B from Toronto Research Chemicals), are provided below.

| Flavonoid | CAS# |
|---|---|
| Cannflavin A | 76735-57-4 |
| Cannflavin B | 76735-58-5 |
| Myricetin | 529-44-2 |
| (−)-Epigallocathechin | 989-51-5 |
| gallate | |
| Polyphenon 60 from green | 138988-88-2 |
| tea | |
| (−)-Gallocathechin | 3371-27-5 |
| Kaempferol | 520-18-3 |
| (±)-Catechin hydrate | 7295-85-4 (anhydrous) |
| Galangin | 548-83-4 |
| Hesperidin | 520-26-3 |
| Baicalein | 491-67-8 |
| Icariin | 489-32-7 |
| Orientin | 28608-75-5 |
| Liquiritigenin | 578-86-9 |
| Acacetin | 480-44-4 |
| Diosmetin | 520-34-3 |
| Scutellarein | 529-53-3 |
| Luteolin | 491-70-3 |

The term “transdermal delivery agent” refers to a substance that aids or facilitates the passage of desired compounds, such as pharmaceutically active ingredients (e.g., external cosmetic agent), cannabinoids, and/or terpenes, at least partially through one or more layers of the skin, including the dermis and epidermis.

The term “antioxidant” refers to an oxidation preventive agent that reduces excessive production of reactive oxygen species. Antioxidants can come from natural products or sources such as vitamin E (tocopherol), vitamin C (ascorbic acid), vitamin A, β-carotene, superoxide dismutase, coenzyme Q. Synthetically derived antioxidants include 3,5-tert-butyl-4-hydroxytoluene (BHT) and 2,3-tert-butyl-hydroxyanisole (BHA) which have been developed to inhibit oxidation of a lipid or the like.

The term “fragrance” refers to a combination of chemicals that gives each product its distinct scent. Chemical ingredients may be derived from synthetic or natural raw materials that can add or mask a scent. The ingredients are capable of imparting or modifying the odor of skin or hair or other substrate.

The term “coloring agent” refers to a natural or synthetic compound that provides a pigment used to enhance the product's appearance and aesthetic value.

The term “exfoliant” refers to an additive used in an attempt to remove dead skin cells exposing the living layer. This provides the skin a smoother softer more youthful appearance, as well as allowing the living layers to grow and stay healthy. Exfoliating topical compositions conventionally use an abrasive compound in a scrubbing motion to remove dead skin cells from the face. Common exfoliants include powders from ground apricot seeds, crushed walnut shells, coconut shells, almond seeds and shells, and sawdust, various solid polymer powders, and various inorganic particles such as sand, salt, alumina, silica, alumino-silicates, lava stone, various phosphates, borates, sulfates, and carbonates.

The term “skin surface” refers to interface of an organism with the environment, which prevents moisture loss from the body, and is a barrier functioning to prevent the invasion of biotoxic substances, such as microorganisms and allergens, from the environment. The skin contains two layers consisting of an outer epidermis and an inner dermis.

The term “human” refers to a person who can benefit from the pharmaceutical formulations and methods of the present invention. The person that could benefit from the presently described pharmaceutical formulations and methods may be an adolescent or adult. A human may be referred to as an individual, patient, subject, or recipient.

The term “topical formulation” is used herein to generally include a formulation that can be applied to skin or a mucosa. Topical formulations may, for example, be used to confer therapeutic benefit to a patient or cosmetic benefits to a consumer. Topical formulations can be used for both topical and transdermal administration of substances. The topical formulations can be configured and formulated to exist in various dosage forms, such as, e.g., gel, pump gel, gel packet, cream, lotion, roll-on liquid, roll-on gel, spray, pump spray, aerosol spray, stick, patch, ointment, liniment, or balm.

The term “topical administration” is used herein to generally include the delivery of a substance, such as a therapeutically active agent (e.g., external cosmetic agent), to the skin or a localized region of the body.

The term “transdermal administration” is used herein to generally include administration through at least a portion of the skin. Transdermal administration is often applied for delivering desired substances to tissues underlying the skin with minimal systemic absorption. As such, the transdermal administration delivers desired substances at least partially through one or more layers of the skin, including the dermis and epidermis.

The term “subject” is used herein to generally include humans, particularly human adolescents (e.g., 12-17 years old) and human adults (e.g., at least 18 years old).

The term “effective amount” is used herein to generally include an amount of topical formulation (or external cosmetic agent) effective for treating or preventing aging skin in a subject as described herein.

The term “treating” with regard to a subject, refers to improving at least one symptom of the subject's aging skin. Treating includes preventing, improving, or at least partially ameliorating the condition or the symptoms thereof.

### Method of Manufacturing

As described herein, the compositions of the present invention are suitable for topical (e.g., dermal or intradermal) administration and include, e.g., liquid or semi-solid preparations (e.g., liniments, lotions, gels, sprays, foams, film forming systems, microneedles, micro- or nano-emulsions), and oil-in-water or water-in-oil emulsions (e.g., creams, ointments or pastes). These topical dosage forms can be prepared employing well-known and routine procedures, equipment, techniques, and substances. See, e.g., A. Williams, Transdermal and Topical Delivery Systems, Pharmaceutical Press, London and Chicago, 2003; L. Lachman, The Theory and Practice of Industrial Pharmacy, 4th Ed., Stipes Publishing, 2015; Remington, Pharmaceutical Sciences, 22^{nd} Rev., Pharmaceutical Press, 2012; H. Benson, Topical and Transdermal Drug Delivery: Principles and Practice 1st Edition, Wiley, 2012; D. Osborne, Topical Drug Delivery Formulations (Drugs and the Pharmaceutical Sciences) 1st Edition, CRC Press, 1989; and M. Brown, The Art and Science of Dermal Formulation Development (Drugs and the Pharmaceutical Sciences) 1st Edition, CRC Press, 2019.

### EXAMPLES:

Production Examples 1-146 provided below illustrate active ingredients (external cosmetic agents) formulated with suitable inactive ingredients to provide various dosage forms (e.g., creams and lotions). As described herein, the formulations illustrated in Production Examples 1-146 can be formulated to further include a cannabinoid, terpene, flavonoid, or combination thereof.

### Production Example 1. Moisturizing Cream for Normal to Dry Skin

Ingredients

Water, glycerin, cetearyl alcohol, caprylic/capric triglyceride, cetyl alcohol, ceteareth-2, petrolatum, potassium phosphate, ceramide np, ceramide ap, ceramide eop, carbomer, dimethicone, behentrimonium methosulfate, sodium lauroyl lactylate, sodium hyaluronate, cholesterol, phenoxyethanol, disodium edta, dipotassium phosphate, tocopherol, phytosphingosine, xanthan gum, ethylhexylglycerin.

### Production Example 2. Moisturizing Cream Unscented

Ingredients

Water, glycerin, petrolatum, dicaprylyl ether, dimethicone, glyceryl stearate, cetyl alcohol, helianthus annuus (sunflower) seed oil, peg-30 stearate, tocopheryl acetate (vitamin E), dimethiconol, acrylates/C10-30 alkyl acrylate crosspolymer benzyl alcohol, phenoxyethanol, prunus amygdalus dulcis (sweet almond) oil, propylene glycol, disodium edta, carbomer, sodium hydroxide

### Production Example 3. Foot Cream

Ingredients

Vetetable glycerin, olive oil, oat flour, lanolin, coconut oil, quillaja extract, oat oil, rosemary leaf oil, tocopherol (vitamin E), fragrance

### Production Example 4. Moisturizing Cream for Normal to Dry Skin

Ingredients

Purified water, glycerin, ceteareth-20 and cetearyl alcohol, caprylic/capric triglyceride, behentrimonium methosulfate, cetearyl alcohol, cetyl alcohol, ceramide 3, ceramide 64-II, ceramide 1, hyaluronic acid, cholesterol, petrolatum, dimethicone, potassium phosphate, dipotassium phosphate, sodium lauroyl lactylate, disodium edta, phenoxyethanol, methylparaben, propylparaben, phytosphingosine, carbomer, xanthan gum

### Production Example 5. Intense Hydration Night Cream

Ingredients

Water, olea europaea (olive) fruit oil, cetyl alchol, stearyl alcohol, glycerin, Ricinus communis (castor) seed oil, tapioca starch, natural fragrance, propanediol, salvia sclarea (clary) extract, lactobacillus, actrocaryum tucuma seed butter, garcinia indica seed butter, trichilia emetica seed butter, beeswax, simmondsia chinensis (jojoba) seed oil, candelilla/jojoba/rice bran polyglyceryl-3 ester, jojoba esters, hydrolyzed jojoba esters, carrageenan, polymnia sonchifolia root juice, alpha-glucan oligosaccharide, maltodextrin, zea mays (corn) starch, sucrose, tocopherol, xanthan gum, glycine soja (soybean) oil, glyceryl stearate, cetearyl alcohol, sodium stearoyl lactylate, sodium PCA, zinc PCA, potassium sorbate, sodium benzoate, phenoxyethanol, amyl cinnamal, eugenol, hydroxycitronellal, linalool

### Production Example 6. Moisturizing Lotion for Nighttime use Ultra-Lightweight Night Cream

Ingredients

Purified water, glycerin, caprylic/capric triglyceride, niacinamide, cetearyl alcohol, ceramide 3, ceramide 6-II, ceramide 1, phytosphingosine, hyaluronic acid, sodium hydroxide, dimethicone, behentrimonium methosulfate, ceteareth-20, polyglyceryl-3 diisostearate, cholesterol, xanthan gum, carbomer, disodium EDTA, dipotassium phosphate, potassium phosphate, sodium lauroyl lactylate, methylparaben, propylparaben

### Production Example 7. Almond and Milk Hand Cream

Ingredients

Prunus Amygdalus Dulcis (Sweet Almond) Oil, Water, Beeswax, Aloe Barbadensis Leaf Juice, Kaolin, Natural Fragrance, Tocopherol, Nonfat Dry Milk, Carrageenan, Glycine Soja (Soybean) Oil, Stearic Acid, Glucose, Sodium Borate, Sodium Chloride, Citric Acid, Sucrose, Lactoperoxidase, Glucose Oxidase, Sodium Benzoate, Potassium Sorbate, Potassium Iodide, Potassium Thiocyanate, Eugenol, Linalool

### Production Example 8. Unscented Moisturizing Skin Cream

Ingredients

Purified Water, Petrolatum, Sorbitol, Cetearyl Alcohol, Propylene Glycol, Ceteareth-20, Simethicone, Glyceryl Stearate, Peg-30 Stearate, Sorbic Acid, Bht.

### Production Example 9. Face and Hand Moisturizing Cream

Ingredients

Water, Mineral Oil, Petrolatum, Glycerin, Microcrystalline Wax, Lanolin Alcohol, Paraffin, Panthenol, Magnesium Sulfate, Decyl Oleate, Octyldodecanol, Aluminum Stearates, Fragrance, Citric Acid, Magnesium Stearate, Methylchloroisothiazolinone, Methylisothiazolinone.

### Production Example 10. Hydrating Body Gel Cream with Hyaluronic Acid

Ingredients

Water, glycerin, isopropyl palmitate, petrolatum, dimethicone, cetyl alcohol, phenoxyethanol, sodium polyacrylate, cetearyl olivate, caprylyl glycol, ethylhexylglycerin, sorbitan, olivate, fragrane, carbomer, sodium hyaluronate, sodium hydroxide, blue 1

### Production Example 11. Cocoa Butter Body Cream for Dry Skin

Ingredients

Water, Mineral Oil, Glycerin, Cetearyl Alcohol, Glyceryl Stearate, Alcohol Denat., Glyceryl Stearate Se, Butyrospermum Parkii (Shea) Butter, Cocos Nucifera (Coconut) Oil, Theobroma Cacao (Cocoa) Seed Butter, Tocopheryl Acetate, Dimethicone, Hydrogenated Cocoglycerides, Sodium Cetearyl Sulfate, Phenoxyethanol, Fragrance, Carbomer, Sodium Hydroxide, Methylparaben, Bht.

### Production Example 12. Hydrating Dry Skin Cream

Ingredients

Water/Agua, Mineral Oil, Isopropyl Palmitate, Petrolatum, Glycerin, Stearic Acid, Ceresin, Glyceryl Stearate, Cetyl Alcohol, Sorbitan Oleate, Candelilla Wax, Triethanolamine, Laureth-23, Fragrance/Fragancia, Carbomer, Disodium Edta, Dmdm Hydantoin, Methylparaben, lodopropynyl Butylcarbamate.

### Production Example 13. Nourishing Moisturizing Cream

Ingredients

Water/Agua, Mineral Oil, Isopropyl Palmitate, Petrolatum, Glycerin, Ceresin, Stearic Acid, Glyceryl Stearate, Cetyl Alcohol, Sorbitan Oleate, Triethanolamine, Laureth-23, Fragrance/Fragancia, Dmdm Hydantoin, Carbomer, Methylparaben, Bht, Tetrasodium Edta, Disodium Edta, Fucus Vesiculosus Extract, Pyrus Malus (Apple) Fruit Extract, Citrus Grandis (Grapefruit) Fruit Extract.

### Production Example 14. Moisturizing Cream Skin Cream

Ingredients

Purified Water, Petrolatum, Sorbitol, Cetearyl Alcohol, Propylene Glycol, Ceteareth-20, Simethicone, Glyceryl Stearate, Peg-30 Stearate, Sorbic Acid.

### Production Example 15. Sensitive Eye Cream

Ingredients water, cetyl alcohol, cetearyl alcohol, glycerin, olea europaea (olive) fruit oil, helianthus annuus (sunflower) seed oil, cocos nucifera (coconut) oil, Cl 77947 (zinc oxide), alcohol denat., kaolin, coco-glucoside, gossypium herbaceum (cotton) flower extract, oryza sativa (rice) extract, beta vulgaris (beet) root extract, artemisia umbelliformis extract, euphrasia officinalis extract, butyrospermum parkii (shea) butter, saccharum officinarum (sugar cane) extract, aloe barbadensis leaf juice, carrageenan, ricinus communis (castor) seed oil, sucrose, fructose, glucose, inositol, citric acid, benzoic acid, lactic acid, trehalose, xanthan gum, potassium sorbate, sodium benzoate, sodium PCA, phenoxyethanol

### Production Example 16. Maximum Hydration 3-in-1 Nourishing Lotion

Ingredients

Water, Glycerin, Isopropyl Palmitate, Alcohol Denat., Glyceryl Stearate SE, C12-15 Alkyl Benzoate, Cetearyl Alcohol, Tapioca Starch, Dimethicone, Glyceryl Glucoside, Sea Salt, Carbomer, Fragrance, Xanthan Gum, Sodium Cetearyl Sulfate, Sodium hydroxide, Phenoxyethanol, Methylparaben, Ethylparaben.

### Production Example 17. Body, Face, and Hand Cream

Ingredients

Water, Glycerin, Myristyl Alcohol, Mineral Oil, Butylene Glycol, Alcohol Denat., Stearic Acid, Petrolatum, Myristyl Myristate, Glyceryl Stearate, Hydrogenated Coco-Glycerides, Dimethicone, Simmondsia Chinensis (Jojoba) Seed Oil, Tocopheryl Acetate, Lanolin Alcohol, Polyglyceryl-2 Caprate, Fragrance, Carbomer, Sodium Hydroxide, Phenoxyethanol.

### Production Example 18. Skin Calming Cream Enriched with Natural Oatmeal

Ingredients

Water, Glycerin, Cetyl Palmitate, Mineral Oil, Caprylic/Capric Triglyceride, Octyldodecanol, Cetyl Alcohol, Glyceryl Stearate, Colloidal Oatmeal, Dimethicone, Methylpropanediol, Peg-40 Stearate, 1, 2-Hexanediol, Phenoxyethanol, Piroctone Olamine, Carbomer, Benzyl Alcohol, Polyglyceryl-2 Caprate, Decylene Glycol.

### Production Example 19. Healing Cream

Ingredients

Water, Petrolatum, Mineral Oil, Ceresin, Lanolin Alcohol, Phenoxyethanol, Piroctone Olamine.

### Production Example 20. Hand Cream

Ingredients

Water, Glycerin, Cetyl Alcohol, Cocos Nucifera (Coconut) Oil, Adansonia Digitata Oil, Caprylic/Capric Triglyceride, Helianthus Annuus (Sunflower) Seed Oil, Fragrance*, Citrus Aurantium Dulcis (Orange) Flower Extract, Pistacia Vera Seed Oil, Citrus Limon (Lemon) Fruit Extract, Butyrospermum Parkii (Shea) Butter, Simmondsia Chinensis (Jojoba) Butter, Theobroma Grandiflorum Seed Butter, Beeswax, Ricinus Communis (Castor) Seed Oil, Hibiscus Rosa-Sinensis Flower Extract, Citrullus Lanatus (Watermelon) Seed Oil, Cucurbita Pepo (Pumpkin) Seed Oil, Carthamus Tinctorius (Safflower) Seed Oil, Acacia Decurrens Flower Wax, Helianthus Annuus (Sunflower) Seed Wax, Tocopherol, Jojoba Esters, Hydrogenated Castor Oil, Glycine Soja (Soybean) Oil, Polyglycerin-3, Biosaccharide Gum-1, Glyceryl Caprylate, Sodium Anisate, Sodium Levulinate, Glyceryl Stearate Citrate, Stearic Acid, Sucrose Stearate, Sodium Stearoyl Lactylate, Magnesium Aluminum Silicate, Sucrose Polystearate, Xanthan Gum, Arginine, Lactic Acid, Phenoxyethanol, Citral, Citronellol, Limonene, Linalool

### Production Example 21. Unscented Dry Hand Cream

Ingredients

Water, Glycerin, Cetearyl Alcohol, Stearic Acid, Sodium Cetearyl Sulfate, Methylparaben, Propylparaben, Dilauryl Thiodipropionate, Sodium Sulfate (702-85)

### Production Example 22. Advanced Repair Hand Cream

Ingredients

Water, Glycerin, Urea, Glyceryl Stearate, Stearyl Alcohol, Cyclomethicone, Dicaprylyl Ether, Sodium Lactate, Dimethicone, Glyceryl Stearate Se, Aluminum Starch Octenylsuccinate, Phenoxyethanol, Pentylene Glycol, Lactic Acid, Xanthan Gum, Ethylhexylglycerin, Sodium Cetearyl Sulfate.

### Production Example 23. Foot Cream—Honey and Bilberry

Ingredients

Water, Vitis Vinifera (Grape) Seed Oil, Simmondsia Chinensis (Jojoba) Butter, Glycerin, Beeswax, Stearic Acid, Theobroma Grandiflorum Seed Butter, Macadamia Ternifolia Seed Oil, Honey, Vaccinium Myrtillus Fruit Extract, Helianthus Annus (Sunflower) Seed Oil, Rosmarinus Officinalis (Rosemary) Leaf Extract, Tocopherol, Beta-Carotene, Xanthan Gum, Canola Oil, Glycine Soja (Soybean) Oil, Glucose, Sucrose Stearate, Sodium Borate, Sodium Chloride, Glucose Oxidase, Lactoperoxidase, Fragrance, Potassium Iodide, Potassium Thiocyanate, Phenoxyethanol, Butylphenyl Methylpropional, Coumarin, Hydroxyisohexyl 3-Cyclohexene Carboxaldehyde

### Production Example 24. Honey and Grapseed Oil Hand Cream

Ingredients

Water, Vitis Vinifera (Grape) Seed Oil, Beeswax, Glycerin, Simmondsia Chinensis (Jojoba) Butter, Stearic Acid, Theobroma Grandiflorum Seed Butter, Macadamia Ternifolia Seed Oil, Honey, Helianthus Annuus (Sunflower) Seed Oil, Rosmarinus Officinalis (Rosemary) Leaf Extract, Beta-Carotene, Tocopherol, Xanthan Gum, Sodium Borate, Sucrose Stearate, Glucose, Glycine Soja (Soybean) Oil, Sodium Chloride, Lactoperoxidase, Glucose Oxidase, Canola Oil, Fragrance, Potassium Iodide, Potassium Thiocyanate, Butylphenyl Methylpropional, Coumarin, Hydroxyisohexyl 3-Cyclohexene Carboxaldehyde

### Production Example 25. Restorative Night Cream

Ingredients

Water, Glycerin, Dimethicone, Ethylhexyl Palmitate, Isononyl Isononanoate, Propylene Glycol, Steareth-2, Butyrospermum Parkii (Shea) Butter, Vinyl Dimethicone/Methicone Silsesquioxane Crosspolymer, Hexylresorcinol, Steareth-21, Ammonium Acryloyldimethyltaurate/Vp Copolymer, Behenyl Alcohol, Phenoxyethanol, Dimethicone Crosspolymer, Ascorbyl Glucoside, Sclerotium Gum, Chlorphenesin, Fragrance, Xylitylglucoside, Anhydroxylitol, Tocopheryl Acetate, Maltodextrin, Disodium Edta, Ethylhexyglycerin, Sodium Hydroxide, Xylitol, Butylene Glycol, Peucedanum Graveolens (Dill) Extract, Rubus Fruticosus (Blackberry) Leaf Extract, Xanthan Gum

### Production Example 26. Lavender and Honey Hand Cream

Ingredients

Water, glycerin, cetyl alcohol, cocos nucifera (coconut) oil, helianthus annuus (sunflower) seed oil, adansonia digitata oil, caprylic/capric triglyceride, natural fragrance, Lavandula angustifolia (lavender) flower extract, honey, butyrospermum parkii (shea) butter, simmondsia chinensis (jojoba) butter, theobroma grandiflorum seed butter, beeswax, Ricinus communis (castor) seed oil, citrullus lanatus (watermelon) seed oil, cucurbita pepo (pumpkin) seed oil, acacia decurrens flower wax, helianthus annuus (sunflower) seed wax, tocopherol, jojoba esters, hydrogenated castor oil, glycine soja (soybean) oil, polyglycerin-3, biosaccaride gum-1, glyceryl caprylate, sodium anisate, sodium levulinate, glyceryl stearate citrate, stearic acid, sucrose stearate, sodium stearoyl lactylate, magnesium aluminum silicate, sucrose polystearate, xanthan gum, arginine, lactic acid, phenoxyethanol, citronellol, coumarin, geraniol, limonene, linalool

### Production Example 27. Leg and Foot Cream

Ingredients

Water, Helianthus Annuus (Sunflower) Seed Oil, Glycerin, Cocos Nucifera (Coconut) Oil, Beeswax, Mentha Piperita (Peppermint) Oil, Stearic Acid, Hamamelis Virginiana (Witch Hazel) Water, Menthol, Sodium Bicarbonate, Melaleuca Alternifolia (Tea Tree) Leaf Oil, Petroselinum Crispum (Parsley) Leaf Oil, Triticum Vulgare (Wheat) Germ Oil, Citrus Aurantium Dulcis (Orange) Peel Wax, Rosmarinus Officinalis (Rosemary) Leaf Extract, Tocopherol, Sucrose Stearate, Glucose, Xanthan Gum, Canola Oil, Sodium Borate, Sodium Chloride, Alcohol, Glucose Oxidase, Lactoperoxidase, Potassium Iodide, Potassium Thiocyanate, Limonene, Linalool

### Production Example 28. Facial Moisturizer

Ingredients

Water, Vinyl Dimethicone/Methicone Silsesquioxane Crosspolymer, Glycerin, Niacinamide*, Isohexadecane, Isopropyl Isostearate, Palmitoyl Pentapeptide-4**, Sodium Hyaluronate, Panthenol***, Dimethicone, Stearyl Alcohol, Sodium Polyacrylate Starch, Cetyl Alcohol, Caprylyl Glycol, 1, 2-Hexanediol, Phenoxyethanol, Behenyl Alcohol, Ceratonia Siliqua (Carob) Fruit Extract, Peg-100 Stearate, Dimethiconol, Cetearyl Glucoside, Cetearyl Alcohol, Fragrance, Stearic Acid, Palmitic Acid, Disodium Edta.

### Production Example 29. Facial Moisturizer

Ingredients

Water, Vinyl Dimethicone/Methicone Silsesquioxane Crosspolymer, Glycerin, Niacinamide*, Isohexadecane, Isopropyl Isostearate, Panthenol**, Camellia Sinensis Leaf Extract***, Dimethicone, Stearyl Alcohol, Sodium Polyacrylate Starch, Cetyl Alcohol, Caprylyl Glycol, 1, 2-Hexanediol, Phenoxyethanol, Inositol, Behenyl Alcohol, Dimethiconol, Peg-100 Stearate, Fragrance, Cetearyl Glucoside, Cetearyl Alcohol, Stearic Acid, Palmitic Acid, Bht, Disodium Edta, Zinc Gluconate, Magnesium Aspartate, Copper Gluconate.

### Production Example 30. Facial Moisturizer

Ingredients

Water, Glycerin, Niacinamide*, Tapioca Starch, Isohexadecane, Isopropyl Isostearate, Stearyl Alcohol, Panthenol**, Tocopheryl Acetate***, Sodium Ascorbyl Phosphate\hat{ }, Camellia Sinensis Leaf Extract\hat{ }\hat{ }, Dimethicone, Cetyl Alcohol, Behenyl Alcohol, Sodium Polyacrylate Starch, Caprylyl Glycol, 1, 2-Hexanediol, Phenoxyethanol, Fragrance, Dimethiconol, Peg-100 Stearate, Cetearyl Glucoside, Cetearyl Alcohol, Polymethylsilsesquioxane, Stearic Acid, Palmitic Acid, Bht, Disodium Edta

### Production Example 31. Intense Hydration Day Lotion

Ingredients

Aqua (Water, Eau), Caprylic/Capric Triglyceride, Glycerin, Cetyl Alcohol, Stearyl Alcohol, Glyceryl Stearate, (Fragrance)*, Propanediol, Salvia Sclarea (Clary) Extract, Lactobacillus, Astrocaryum Tucuma Seed Butter, Trichilia Emetica Seed Butter, Cera Alba (Beeswax, Cire D'abeille), Simmondsia Chinensis (Jojoba) Seed Oil, Aloe Barbadensis Leaf Powder, Persea Gratissima (Avocado) Oil, Citrus Aurantium Amara (Bitter Orange) Peel, Polymnia Sonchifolia Root Juice, Lecithin, Alpha-Glucan Oligosaccharide, Maltodextrin, Tocopherol, Xanthan Gum, Glycine Soja (Soybean) Oil, Stearic Acid, Sodium Pca, Potassium Stearate, Potassium Sorbate, Sodium Benzoate, Phenoxyethanol, Amyl Cinnamal, Eugenol, Hydroxycitronellal, Linalool

### Production Example 32. Firming Moisturizing Cream

Ingredients

Water, Glycerin, Helianthus Annuus (Sunflower) Seed Oil, Caprylic/Capric Triglyceride, Decyl Cocoate, Coco-Caprylate, Decyl Oleate, Cetyl Alcohol, Stearic Acid, Beeswax, Glyceryl Stearate Citrate, Fragrance+, Stearyl Alcohol, Hibiscus Sabdariffa Flower Extract, Pyrus Malus (Apple) Fruit Extract, Citrullus Lanatus (Watermelon) Fruit Extract, Lens Esculenta (Lentil) Fruit Extract, Scutellaria Alpina Flower/Leaf/Stem Extract, Adansonia Digitata Fruit Extract, Trifolium Pratense (Clover) Leaf Extract, Plankton Extract, Beta Vulgaris (Beet) Root Extract, Crambe Abyssinica Seed Oil, Phragmites Communis Extract, Poria Cocos Extract, Ribose, Tocopherol, Arginine, Dipotassium Glycyrrhizate, Sclerotium Gum, Linoleic Acid, Glycine Soja (Soybean) Oil, P-Anisic Acid, Zea Mays (Corn) Starch, Coco-Glucoside, Hydrolyzed Corn Starch, Magnesium Aluminum Silicate, Xanthan Gum, Sodium Stearoyl Lactylate, Cetearyl Alcohol, Sodium Lactate, Sodium Pca, Citric Acid, Sodium Citrate, Lactic Acid, Linolenic Acid, Sodium Benzoate, Potassium Sorbate, Phenoxyethanol, Eugenol, Limonene, Linalool

### Production Example 33. Anti-Wrinkle+Firming Night Cream

Ingredients

Water, Glycerin, Dimethicone, Hydrogenated Polyisobutene, Cetyl Alcohol, Glyceryl Stearate, Aluminum Starch Octenylsuccinate, Peg-40 Stearate, Prunus Armeniaca Kernel Oil/Apricot Kernel Oil, Shorea Robusta Butter/Shorea Robusta Seed Butter, Paraffinum Liquidum/Mineral Oil, Paraffin, Zea Mays Extract/Corn Kernel Extract, Stearic Acid, Stearyl Alcohol, Cera Microcristallina Wax, Sorbitan Tristearate, Soybean Protein, Triethanolamine, Isohexadecane, Myristyl Alcohol, Palmitic Acid, Phenethyl Alcohol, Hydrolyzed Soy Protein, Caprylyl Glycol, Acetyl Trifluoromethylphenyl Valylglycine, Yeast Extract, Polysorbate 80, Acrylamide/Sodium Acryloyledimethyltaurate Copolymer, Retinyl Palmitate, Potassium Sorbate, Sodium Benzoate, Phenoxyethanol, Linalool, Geraniol, Alpha-Isomethyl Ionone, Amyl Cinnamal, Limonene, Citral, Citronellol, Butyphenyl Methylpropional, Hexyl Cinnamal, Benzyl Alcohol, Benzyl Benzoate, Benzyl Cinnamate, Benzyl Salicylate, Fragrance

### Production Example 34. Anti-Aging and Firming Night Cream

Ingredients

Aqua/Water, Glycerin, Rhamnose, Prunus Armeniaca Kernel Oil, Apricot Kernel Oil, Caprylic/Capric Triglyceride, Pentaerythrityl Tetraethylhexanoate, Cetyl Alcohol, Zea Mays Starch, Corn Starch, Butyrospermum Parkii Butter, Shea Butter, Petrolatum, Glyceryl Stearate, Pentylene Glycol, Dimethicone, Peg-40 Stearate, Cera Alba, Beeswax, Hydroxyethylpiperazine Ethane Sulfonic Acid, Stearyl Alcohol, Sorbitan Tristearate, Dimethiconol, Triethanolamine, Myristyl Alcohol, Phenoxyethanol, Adenosine, Magnesium Ascorbyl Phosphate, Ascorbyl Glucoside, Poloxamer 338, Caprylyl Glycol, Xanthan Gum, Pentaerythrityl Tetra-Di-T-Butyl Hydroxyhydrocinnamate, Parfum/Fragrance

### Production Example 35. Anti-Wrinkle+Firming Face/Neck Contour Cream

Ingredients

Aqua/Water, Cyclohexasiloxane, Glycerin, Paraffinum Liquidum/Mineral Oil, Myristyl Myristate, Shorea Robusta Butter/Shorea Robusta Seed Butter, Stearic Acid, Palmitic Acid, Peg-100 Stearate, Cera Alba/Beeswax, Glyceryl Stearate, Peg-20 Stearate, Stearyl Alcohol, Glycine Soja Protein/Soybean Protein, Triethanolamine, Isohexadecane, Phenethyl Alcohol, Hydrolyzed Soy Protein, Acetyl Trifluoromethylphenyl Valylglycine, Faex Extract/Yeast Extract, Polysorbate 80, Acrylamide/Sodium Acryloyldimethyltaurate Copolymer, Acrylates Copolymer, Cetyl Alcohol, Retinyl Palmitate, Potassium Sorbate, Methylparaben, Sodium Benzoate, Chlorhexidine Digluconate, Ethylparaben, Linalool, Geraniol, Alpha-Isomethyl Ionone, Amyl Cinnamal, Limonene, Citronellol, Butylphenyl Methylpropional, Hexyl Cinnamal, Benzyl Alcohol, Benzyl Benzoate, Benzyl Salicylate, Parfum/Fragrance, Phenoxyethanol

### Production Example 36. Rapid Tone Retinol Hyaluronic Acid Night Cream

Ingredients

Water, Pentaerythrityl Tetraethylhexanoate, Dimethicone, Glycerin, Ppg-15 Stearyl Ether, Stearyl Alcohol, Cetearyl Alcohol, Butylene Glycol, Isohexadecane, Ceteareth 20, Retinol, Bht, Hydrolyzed Myrtus Communis Leaf Extract, Ascorbic Acid, Trisiloxane, Dimethicone Crosspolymer, Caprylyl Glycol, Sodium Hyaluronate, Polysorbate 20, Ammonium Acryloldimethyltaurate/Vp Copolymer, Polyacrylamide, Polyethylene, C13-14 Isoparaffin, Ptfe, Disodium Edta, Laureth-7, Sodium Hydroxide, Phenoxyethanol, Chlorphenesin, Fragrance

### Production Example 37. Hydrating Beauty Fluid Lotion

Ingredients

Water, Glycerin, Cetyl Palmitate, Mineral Oil , Petrolatum, Cetyl Alcohol, Glyceryl Hydroxystearate, Steareth-100, Octyldodecyl Myristate, Dimethicone, Stearic Acid, Palmitic Acid, Fragrance, Potassium Hydroxide, DMDM Hydantoin, Tetrasodium EDTA, Acrylates/C10-30 Alkyl Acrylate Crosspolymer, Carbomer, lodopropynyl Butylcarbamate, Red 4

### Production Example 38. Skin Renewing Night Cream to Soften Skin

Ingredients

Purified Water, Glycerin, Biosaccharide Gum-1, Caprylic/Capric Triglyceride, Butyrospermum Parkii (Shea Butter), Dimethicone/Vinyl Dimethicone Crosspolymer, Glyceryl Stearate Se, Stearic Acid, Cyclopentasiloxane, Cetyl Alcohol, Dimethicone, Saccharide Isomerate, Glycine Soja (Soybean) Sterols, Ceramide 6 11, Ceramide 3, Ceramide 1, Niacinamide, Hyaluronic Acid, Caprooyl Tetrapeptide-3, Tripeptide-1, Cholesterol, Phenoxyethanol, Cetearyl Alcohol, Carbomer, Behentrimonium Methosulphate, Sodium Hydroxide, Tocopherol, Allantoin, Ethylhexylglycerin, Butylene Glycol, Sodium Lauroyl Lactylate, Phytosphingosine, Dextran, Xanthan Gum

### Production Example 39. Collagen Moisture Filler Day/Night Cream

Ingredients

691418/10 Ater Cyclohexasiloxane Glycerin Butyrospermum Parkii Butter/Shea Butter Isohexadecane Pentaerythrityl Tetraethylhexanoate Dipropylene Glycol Cera Alba/Beeswax Stearic Acid

### Production Example 40. Deep Moisture Night Cream with Glycerin & Shea Butter

Ingredients

Water, Glycerin, Cetearyl Alcohol, Dicaprylyl Carbonate, Stearic Acid, Butyrospermum Parkii (Shea) Butter, Silica, Bht, Petrolatum, Ethylhexyl-Glycerin, Cholecalciferol, Calcium Chloride, Caprylic/Capric Triglyceride, Sodium Polyacrylate, Caprylyl Glycol, Phenoxyethanol, Fragrance

### Production Example 41. Oil-Free Matifying Moisturizer

Ingredients

Water, Cyclomethicone, Butylene Glycol, SD Alcohol 40-B, Glycerin, Silica, Xylitol, Pentaerythrityl Tetraoctanoate, PEG-6, PEG-32, Xanthan Gum, Magnesium Chloride, Peony Root Extract, Calcium Chloride, Eysenhardtia Polystachya (Palo Azul) Wood Extract, Rosemary Extract, Glyceryl Stearate Se, PEG-60 Glyceryl Isostearate, Carbomer, Acrylates/C10-30 Alkyl Acrylate Crosspolymer, Potassium Hydroxide, Trisodium EDTA, Tocopherol, Methylparaben, Phenoxyethanol, Benzoic Acid, Fragrance.

### Production Example 42. Wrinkle Repair Hyaluronic Acid & Retinol Cream

Ingredients

Water, Pentaerythrityl Tetraethylhexanoate, Glycerin, Dimethicone, Ppg-15 Stearyl Ether, Stearyl Alcohol, Cetearyl Alcohol, Butylene Glycol, Glyceryl Behenate, Ceteareth-20, Isohexadecane, Cyclopentasiloxane, Dimethicone Crosspolymer, Polyethylene, Sodium Polyacrylate, Phenoxyethanol, Caprylyl Glycol, Sodium Acryloyldimethyltaurate/Vp Crosspolymer, Polyacrylamide, Fragrance, C13-14 Isoparaffin, Ptfe, Chlorphenesin, Polysorbate 20, Hydrolyzed Myrtus Communis Leaf Extract, Retinol, Bht, Sodium Hyaluronate, Disodium Edta, Laureth-7, Sodium Hydroxide, Ascorbic Acid.

### Production Example 43. Recovery Night Cream

Ingredients

Hydrogenated C6-14 Olefin Polymers, Glycerin, Alcohol Denat, Diproylene Glycol, Cetyl PEG/PPG-10/1 Dimethicone.

### Production Example 44. Anti-Wrinkle Deep Wrinkle Night Moisturizer

Ingredients

Water, Ppg-2 Myristyl Ether Propionate, Cetearyl Alcohol, Cetyl Dimethicone, Pentaerythrityl Tetraethylhexanoate, Hexadecyl Stearate, Caprylyl Methicone, Dimethicone, Behenyl Alcohol, Ethylhexylglycerin, Squalane, Glycerin, Shea Butter (Butyrospermum Parkii), Bht, Panthenol, Allantoin, Arginine, Erythorbic Acid, Tocopherol, Retinol, Polysorbate 20, Sodium Hyaluronate, Dipotassium Glycyrrhizate, Bisabolol, C13-14 Isoparaffin, Laureth-7, Cetearyl Glucoside, Glyceryl Stearate, Peg-100 Stearate, Polyacrylamide, Nylon-12, Disodium Edta, Propylparaben, Ethylparaben, Methylparaben.

### Production Example 45. Glowing Water Cream Normal/Dry Skin

Ingredients

Aqua/Water, Glycerin, Dimethicone, Cetearyl Ethylhexanoate, Alcohol Denat., Hydroxyethylpiperazine Ethane Sulfonic Acid, Silanetriol, Bisabolol, Dimethiconol, Aloe Barbadensis Leaf Juice, Sodium Hyaluronate, Sodium Hydroxide, Silica Dimethyl Silylate, Hyaluronic Acid, Ascorbyl Glucoside, Ammonium Polyacryloyldimethyl Taurate, Disodium Edta, Isopropyl Myristate, Caprylyl Glycol, Citric Acid, Xanthan Gum, T-Butyl Alcohol, Betula Alba Juice, Ethylhexyl Palmitate, Butylene Glycol, Hexylene Glycol, Tocopherol, Potassium Sorbate, Sorbic Acid, Sodium Benzoate, Phenoxyethanol, Ci 42090/Blue 1, Linalool, Farnesol, Limonene, Citral, Parfum/Fragrance

### Production Example 46. Daily Renewal Cream Facial Moisturizer

Ingredients

Water, Ppg-15 Stearyl Ether, Glycerin, Stearyl Alcohol, Salicylic Acid, Cetyl Alcohol, Aloe Barbadensis Leaf Extract, Acrylamide/Sodium Acryloyldimethyltaurate Copolymer, Dimethicone, Steareth-21, C13-14 Isoparaffin, Cyclopentasiloxane, Laureth-7, Dimethiconol, Tocopheryl Acetate, Steareth-2, Disodium Edta, Fragrance.

### Production Example 47. Rich Hydrating Night Cream

Ingredients

Water, Hydrogenated Polydecene, Caprylic/Capric Triglyceride, Dimethicone, Glycerin, Butylene Glycol, Cetearyl Olivate, Sorbitan Olivate, Cyclopentasiloxane, Marrubium Vulgare Extract, Hydrolyzed Hyaluronic Acid, Olea Europaea (Olive) Fruit Oil, Tocopheryl Acetate (Vitamin E), Ascorbyl Palmitate (Vitamin C), Leuconostoc/Radish Root Ferment Filtrate, Phospholipids, Isopropyl Lauroyl Sarcosinate, Cetearyl Alcohol, Sodium Polyacrylate, 1, 2-Hexanediol, Caprylyl Glycol, Polymethylsilsesquioxane, Isohexadecane, Glyceryl Stearate, Peg-100 Stearate, Cetearyl Methicone, Phenoxyethanol, Peg-40 Stearate, Steareth-2, Steareth-21, Retinyl Palmitate, Linoleic Acid, Lycopene

### Production Example 48. Anti Fatigue Night Cream and Face Mask with Hyaluronic Acid

Ingredients

Aqua/water, glycerin, dimethicone, caprylic/capric triglyceride, squalene, ammonium polyacryldimethyltauramide/ammonium polyacryloyldimethyl taurate, ci 42090/blue 1, ci 42053/green 3, dimethiconol, sodium hyaluronate, phenoxyethanol, poloxamer 338, disodium edta, caprylyl glycol, citric acid, biosaccharide gum-1, xanthan gum, parfum/fragrance

### Production Example 49. Renewal Firming Eye Cream

Ingredients

Water, Caprylic/Capric Triglyceride, Glycerin, Stearyl Alcohol, Cetyl Alcohol, Glyceryl Stearate, Beeswax, Jbakuchiol, Tocopherol, Beta Vulgaris (Beet) Root Extract, Citrullus Lanatus (Watermelon) Fruit Extract, Lens Esculenta (Lentil) Fruit Exiract, Pyrus Malus (Apple) Fruit Extract, Glycine Soja (Soybean) Oil, Scutellaria Alpina Flower/Leaf/Stem Extract, Trifolium Pratense (Cover) Leaf Extract, Adansonia Digitata Fruit Extract, Hibiscus Sabdariffa Flower Extract, Chlorella Vulgaris Extract, Jplankton Extract, Phragmites Communis Extract, Poria Cocos Extract, Stearic Acid, Ribose, Xanthan Gum, Lecithin, Hydrolyzed Corn Starch, Citric Acid, Dipotassium Glycyrrhizate, P-Anisic Acid, Potassium Stearate, Linoleic Acid, Linolenic Acid, Lactic Acid, Magnesium Aluminum Silicate, Sodium Lactate, Sodium Pca, Sodium Citrate, Sodium Benzoate, Potassium Sorbate, Phenoxyethanol

### Production Example 50. Night Cream with Vitamin B3

Ingredients

Water, Octyldodecyl Neopentanoate, Dicaprylyl Carbonate, Glycerin, Glycine Soja (Soybean) Seed Extract, Mangifera Indica (Mango) Seed Butter, Behenyl Alcohol, Candelilla/Bojoba/Rice Bran Polyglyceryl-3 Esters, Glyceryl Stearate, Niacinamide Potassium Cetyl Phosphate, Vinyl Dimethicone/Methicone Silsesquioxane Crosspolymer, Phenoxyethanol, Cetearyl Alcohol, Sodium Stearoyl Lactylate, Microcrystalline Cellulose, Caprylyl Glycol, Fragrance, Magnesium Aluminum Silicate, Chlorphenesin, Cetyl Hydroxyethylcellulose, Disodium Edta, Sodium Pca, Urea, Tocopheryl Acetate, Ethylhexylglycerin, Cellulose Gum, Trehalose, Glyceryl Acrylate/Acrylic Acid Copolymer, Hexylene Glycol, Polyquaternium-51, Triacetin, Sodium Hyaluronate

### Production Example 51. Enriched Revitalizing Cream

Ingredients

Ingredients: Aqua (Water), Glycerin, Hydrogenated C6-14 Olefin Polymers, Petrolatum, Cetyl Octanoate, Dipropylene Glycol, Squalane, Microcrystalline Wax, Maltitol, Dimethicone, Disteardimonium Hectorite, PEG-150, Polyglyceryl-2 Diisostearate, Sodium PCA, Tocopheryl Acetate, Potassium Ascorbyl Tocopheryl Phosphate, Arginine HCI, Ginseng Extract, Sodium Acetylated Hyaluronate, Hydroxyproline, Chlorella Vulgaris Extract, Polyvinyl Alcohol, PEG-12 Dioleate, Butylene Glycol, Trisodium EDTA, Alcohol, Sodium Hexametaphosphate, Tocopherol, Ethylparaben, Butylparaben, Fragrance, Iron Oxides.

### Production Example 52. Moisturizing Gel Cream

Ingredients

Aqua, Glycerin, Butylene Glycol, Dimethicone, SD Alcohol 40-B, Xylitol, Pentaerythrityl Tetraoctanoate, Hydrogenated C6-14 Olefin Polymers, Corn Starch, Eysenhardtia Polystachya (Palo Azul) Wood Extract, Potassium Carbomer, Xanthan Gum, Dimethicone Copolyol, Potassium-Acrylates/C10-30 Alkyl Acrylate Crosspolymer, Sucrose Stearate, Trisodium EDTA, Sodium Hexametaphosphate, Magnesium Chloride, Calcium Chloride, Peony Root Extract, Rosemary Extract, Methylparaben, Benzoic Acid, Fragrance, Iron Oxides.

### Production Example 53. Night Cream

Ingredients

Water, Glycerin, Hydrogenated C6-14 Olefin Polymers, Petrolatum, Cetyl Octanoate, Dipropylene Glycol, Squalane, Microcrystalline Wax, Maltitol, Dimethicone, Disteardimonium Hectorite, PEG-150, Polyglyceryl-2 Diisostearate, Sodium PCA, Tocopheryl Acetate, Potassium Ascorbyl Tocopheryl Phosphate, Arginine HCI, Ginseng Extract, Sodium Acetylated Hyaluronate, Hydroxyproline, Chlorella Vulgaris Extract, Polyvinyl Alcohol, PEG-12 Dioleate, Butylene Glycol, Trisodium EDTA, Alcohol, Sodium Hexametaphosphate, Tocopherol, Ethylparaben, Butylparaben, Fragrance, Iron Oxides.

### Production Example 54. Facial Moisturizer

Ingredients

Water, Butylene Glycol, Niacinamide*, Pentylene Glycol, Dipropylene Glycol, Panthenol**, Sodium Hyaluronate***, Palmitoyl Pentapeptide-4\hat{ }, Aloe Barbadensis Leaf Extract, Camellia Sinensis Leaf Extract, Tremella Fuciformis Sporocarp Extract\hat{ }\hat{ }, Sodium Peg-7 Olive Oil Carboxylate, Hydrolyzed Elastin, Glycerin, Glyceryl Acrylate/Acrylic Acid Copolymer, Glycereth-25 Pca Isostearate, Disodium Edta, Pvm/Ma Copolymer, Sodium Citrate, Benzyl Alcohol, Methylparaben, Ethylparaben, Fragrance

### Production Example 55. Face Moisturizer with Green Tea, Oily Skin

Ingredients

Aqua/Water, Glycerin, Dicaprylyl Ether, Alcohol Denat., Caprylic/Capric Triglyceride, Zea Mays Starch/Corn Starch, Polyglyceryl-3 Methylglucose Distearate, Stearyl Alcohol, Glyceryl Stearate Se, Carbomer*, Salicylic Acid*, Caprylyl Glycol*, Tocopherol, Parfum/Fragrance*, Sodium Hydroxide*, Xanthan Gum, Sodium Phytate, Glycine Soja Oil/Soybean Oil, Linalool*, Maltodextrin, Benzyl Salicylate*, Camellia Sinensis Leaf Extract, Limonene*, Dextrin, Benzyl Alcohol*, Geraniol*.

### Production Example 56. Water Gel Face Moisturizer

Ingredients

Water, Dimethicone, Glycerin, Dimethicone/Vinyl Dimethicone Crosspolymer, Phenoxyethanol, Polyacrylamide, Cetearyl Olivate, Sorbitan Olivate, Dimethiconol, C13-14 Isoparaffin, Dimethicone Crosspolymer, Chlorphenesin, Carbomer, Laureth-7, Sodium Hyaluronate, Ethylhexylglycerin, Fragrance, C12-14 Pareth-12, Sodium Hydroxide, Blue 1

### Production Example 57. Unscented Hyaluronic Acid Gel Face Moisturizer to Hydrate and Smooth Extra-Dry Skin

Ingredients

Water, Dimethicone, -Glycerin, Cetearyl Olivate, Polyacrylamide, Sorbitan Olivate, Phenoxyethanol, Dimethicone/Vinyl, Dimethicone Crosspolymer, Chlorphenesin, Laureth-7, Carbomer, Sodium Hyaluronate, Ethylhexylglycerin, C12-14 Pareth -12, Sodium Hydroxide

### Production Example 58. Cicacream Anti-Wrinkle+Skin Barrier Repair

Ingredients

Aqua/Water, Glycerin, C12-15 Alkyl Benzoate, Caprylic/Capric Triglyceride, Sesamum Indicum Seed Oil/Sesame Seed Oil, Glyceryl Stearate, Butyrospermum Parkii Butter/Shea Butter, Peg-100 Stearate, Sorbitan Stearate, Theobroma Cacao Seed Butter/Cocoa Seed Butter, Cetearyl Alcohol, Phenoxyethanol, Methyl Dihydrojasmonate, Stearic Acid, Carbomer, Tocopheryl Acetate, Palmitic Acid, Chlorphenesin, Caprylyl Glycol, Ceteareth-20, Xanthan Gum, Polysorbate 20, Sodium Hydroxide, Dimethicone, Pentaerythrityl Tetra-Di-T-Butyl Hydroxyhydrocinnamate, Retinyl Palmitate, Disodium Edta, Madecassoside, Myristic Acid, Faex Extract/Yeast Extract/Extrait De Levure, Dimethiconol, Centella Asiatica Extract, Ethylhexylglycerin, Tocopherol, Bht, Citric Acid

### Production Example 59. Nourishment Eye Cream

Ingredients

Water, Glycerin, Helianthus Annuus (Sunflower) Seed Oil, Cetyl Alcohol, Cetearyl Alcohol, Olea Europaea (Olive) Fruit Oil, Butyrospermum Parkii (Shea) Butter, Coco-Glucoside, Glyceryl Rosinate, Lonicera Japonica (Honeysuckle) Flower Extract, Lonicera Caprifolium (Honeysuckle) Flower Extract, Calendula Officinalis Flower Extract, Hydrastis Canadensis (Goldenseal) Extract, Hamamelis Virginiana (Witch Hazel) Leaf Extract, Royal Jelly, Xanthan Gum, Olea Europaea (Olive) Oil Unsaponifiables, Tocopherol, Citric Acid, Glycine Soja (Soybean) Oil, Phenoxyethanol

### Production Example 60. Rapid Wrinkle Repair Eye Cream with Hyaluronic Acid

Ingredients

Water, Pentaerythrityl Tetraethylhexanoate, Dimethicone, Glycerin, Ppg-15 Stearyl Ether, Cetearyl Alcohol, Stearyl Alcohol, Butylene Glycol, Ceteareth-20, Isohexadecane, Trisiloxane, Dimethicone Crosspolymer, Phenoxyethanol, Caprylyl Glycol, Ammonium Acryloyldimethyltaurate/Vp Copolymer, Polyacrylamide, Polyethylene, Chlorphenesin, C13-14 Isoparaffin, Ptfe, Hydrolyzed Myrtus Communis Leaf Extract, Bht, Sodium Hyaluronate, Disodium Edta, Laureth-7, Ascorbic Acid, Polysorbate 20, Retinol, Sodium Hydroxide

### Production Example 61. Skin Renewing Retinol Face Cream Serum for Fine Lines and Wrinkles

Ingredients

Purified Water, Glycerin, Caprylic/Capric Triglyceride, Potassium Cetyl Phosphate, Hydrogenated Palm Glycerides, Polysorbate 20, Butyrospermum Parkii (Shea) Butter, Peg-40 Stearate, Hydroxyethylcellulose, Cyclopentasiloxane, Dimethicone/Vinyldimethicone Crosspolymer, Triethanolamine, Cetearyl Alcohol, Pentylene Glycol, Lecithin, Xanthan Gum, Hydroxyacetophenone, Phenoxyethanol, Citric Acid, Behentrimonium Methosulphate, Cichorium Intybus (Chicory) Root Extract, Tocopherol, Niacinamide, Alcohol, Retinol, Ethylhexylglycerin, Silica, Potassium Phosphate, Sodium Lauroyl Lactylate, Hyaluronic Acid, Sodium Hydroxide, Ceramide 3, Ceramide 6 Ii, Phytosphingosine, Cholesterol, Carbomer, Ceramide 1

### Production Example 62. Hydrating Eye Gel Cream

Ingredients

Water, Cyclopentasiloxane, Dimethicone Crosspolymer, Glycerin, Hdi/Trimethylol Hexyllactone Crosspolymer, Butylene Glycol, Octyldodecanol, Dihydroxyisopropyl Capryloylcaprylamide, Niacinamide, Polysorbate 20, Sodium Hyaluronate, Sodium Polyacrylate, Tocopheryl Acetate, Portulaca Oleracea Extract, Crithmum Maritimum Extract, Glycyrrhiza Glabra (Licorice) Root Extract, Camellia Sinensis Leaf Extract, Poria Cocos Extract, Vitis Vinifera (Grape) Seed Oil, Camellia Sinensis Seed Oil, Limnanthes Alba (Meadowfoam) Seed Oil, Hydrogenated Vegetable Oil, Sorbitan Olivate, Sucrose Cocoate, Caprylyl Glycol, Bisabolol, Dimethicone, Cetyl Palmitate, Sorbitan Palmitate, Serine, Phytosterols, Alanine, Glycine, Trideceth-6, Caprylic/Capric Triglyceride, Sodium Carboxymethyl Beta-Glucan, Myristoyl/Palmitoyl Oxostearamide/Arachamide Mea, Biotinoyl Hexapeptide-2 Amide, Ammonium Acryloyldimethyltaurate/Vp Copolymer, Peg/Ppg-18/18 Dimethicone, Xanthan Gum, Silica, Glyceryl Stearate, Sorbitan Stearate, Phenoxyethanol, Polyvinyl Alcohol, Cetearyl Alcohol, Stearic Acid, Citric Acid, Fragrance

### Production Example 63. Pro-Retinol Eye Cream Treatment

Ingredients

Water/Eau, Glycerin, Niacinamide, C12-15 Alkyl Benzoate, Polyethylene, Dimethicone, Stearyl Alcohol, Cetyl Palmitate, Tocopheryl Acetate, Panthenol, Carnosine, Cetyl Alcohol, Caprylic/Capric Triglyceride, Behenyl Alcohol, Sodium Acrylate/Sodium Acryloyldimethyl Taurate Copolymer, Isohexadecane, Retinyl Propionate, Cetyl Ricinoleate, Dmdm Hydantoin, Carbomer, Polysorbate 80, Stearic Acid, Polysorbate 20, Peg-100 Stearate, Disodium Edta, Sodium Hydroxide, Bht, lodopropynyl Butylcarbamate.

### Production Example 64. Brightening Eye Treatment

Ingredients

Water, Helianthus Annuus (Sunflower) Seed Oil, Prunus Armeniaca (Apricot) Kernel Oil, Butyrospermum Parkii (Shea) Butter, Olea Europaea (Olive) Fruit Oil, Glyceryl Stearate Citrate, Cetyl Alcohol, Glycerin, Bellis Perennis (Daisy) Flower Extract, Leucojum Aestivum Bulb Extract, Euphrasia Officinalis Extract, Lactobacillus/Dipteryx Odorata Seed Ferment Filtrate, Leuconostoc/Radish Root Ferment Filtrate, Quillaja Saponaria Bark Extract, Aesculus Hippocastanum (Horse Chestnut) Seed Extract, Emblica Officinalis Fruit Powder, Melissa Officinalis Leaf Extract, Vitis Vinifera (Grape) Seed Oil, Aloe Barbadensis Leaf Extract, Xanthan Gum, Maltodextrin, Arginine, Alcohol Denat., Phenethyl Alcohol, Fragrance**, Sodium Benzoate, Phenoxyethanol, Citral, Limonene, Linalool

### Production Example 65. Dark Spot Corrector

Ingredients

Ingredients: Water, Helianthus Annuus (Sunflower) Seed Oil, Glycerin, Silica, Bakuchiol, Sodium Ascorbyl Phosphate*, Ascophyllum Nodosum Extract, Tocopherol, Citric Acid, Gellan Gum, Xanthan Gum, Lauryl Glucoside, Myristyl Glucoside, Polyglyceryl-6 Laurate, Fragrance **, Potassium Sorbate, Sodium Benzoate, Phenoxyethanol, Eugenol, Geraniol, Limonene, Linalool

### Production Example 66. Hyaluronic Acid Eye Gel

Ingredients

Aqua/Water/Eau, Propanediol, Butyrospermum Parkii Butter/Shea Butter, Glycerin, Carbomer, Caffeine, Sodium Hyaluronate, Adenosine, Phenoxyethanol, Chlorella Vulgaris Extract, Citric Acid, Caprylyl Glycol, Biosaccharide Gum-1.

### Production Example 67. Honey Eye Gel

Ingredients

Aqua/Water/Eau, Glycerin, Propanediol, Alcohol Denat., Pentylene Glycol, Mel/Honey/Miel, PEG-60 Hydrogenated Castor Oil, Caffeine, Phenoxyethanol, Triethanolamine, Carbomer, Mel Extract/Honey Extract, Sodium Hyaluronate, Tocopherol, Ammonium Polyacryloyldimethyl Taurate, Olea Europaea Oil/Olive Fruit Oil, Camelina Sativa Seed Oil, Sim Mondsia Chinensis Seed Oil/Jojoba Seed Oil, Cocos Nucifera Oil/Coconut Oil, Caprylic/Capric Triglyceride, Caramel, Steareth-20, Calcium pca , Chlorhexidine Digluconate, Citric Acid , N-Hydroxysuccinimide, Potassium Sorbate, Palmitoyl Oligopeptide, Chrysin, Palmitoyl Tetrapeptide-7

### Production Example 68. Awakening Eye Balm Facial Treatment

Ingredients

Aqua, Glycerin, Dimethicone, Propanedial, Dimethicone/Peg-10/15 Crosspolymer, Ci 42090, Caffeine, Sodium Chloride, Sodium Citrate, Sodium Hyaluronate, Sodium Hydroxide, Phenoxyethanol, P-Anisic Acid, Chondrus Crispus (Carrageenan), Disodium Edta, Citric Acid, Biosaccharide Gum-1, Polymethylsilsesquioxane

### Production Example 69. Retinol Anti-Wrinkle Cream

Ingredients

Aqua/Water, Glycerin, Dicaprylyl Ether, Isopropyl Isostearate, Octyldodecanol, Sodium Tetrahydro Jasmonate, Glycine Soja Oil/Soybean Oil, Cetyl Alcohol, Propylene Glycol, Behenyl Alcohol, Peg-100 Stearate, Stearyl Alcohol, Carbomer, Arachidyl Alcohol, Cetearyl Alcohol, Cetearyl Glucoside, Retinol, Sodium Hyaluronate, Sodium Hydroxide, Phenoxyethanol, Adenosine, Ammonium Polyacryldimethyltauramide/Ammonium Polyacryloyldimethyl Taurate, Caprylyl Glycol, Parfum/Fragrance

### Production Example 70. Double Lifting Day Treatment

Ingredients

Aqua/Water, Glycerin, Cyclohexasiloxane, Alcohol Denat, Isocetyl Stearate, Butylene Glycol, Lithium Magnesium Sodium Silicate, Peg-100 Stearate, Peg-20 Stearate, Stearic Acid, Stearyl Alcohol, Linalool, Cera Alba/Beeswax, Geraniol, Glyceryl Stearate, Glycine Soja Protein/Soybean Protein, Triethanolamine, Isohexadecane, Helianthus Annuus Seed Oil/Sunflower Seed Oil, Silica, Coumarin, Limonene, Citral, Citronellol, Acetyl Trifluoromethylphenyl Valylglycine, Polymethyl Methacrylate, Polysorbate 80, Polyurethane-2, Acrylamide/Sodium Acryloyldimethyltaurate Copolymer, Cetyl Alcohol, Butylphenyl Methylpropional, Benzyl Salicylate, Retinyl Palmitate, Methylparaben, Chlorhexidine Digluconate, Caprylyl Glycol, Ethylparaben, Benzyl Alcohol, Ci 14700/Red 4, Ci 17200/Red 33, Parfum/Fragrance, Alpha-Isomethyl Ionone, Hydroxyisohexyl 3-Cyclohexene Carboxaldehyde, Sodium Dehydroacetate, Phenoxyethanol, Chlorphenesin

### Production Example 71. Anti-Wrinkle and Firming Daily Moisture

Ingredients

Aqua/Water, Glycerin, Cyclohexasiloxane, Caprylic/Capric Triglyceride, Dimethicone, Aluminum Starch Octenylsuccinate, Cyclopentasiloxane, PEG-100 Stearate, Glyceryl Stearate, Butyrospermum Parkii Butter/Shea Butter, Mentha Piperita Extract/Peppermint Leaf Extract, C12-13 Pareth-23, C12-13 Pareth-3, Stearic Acid, Triethanolamine, Dimethiconol, Lithium Magnesium Sodium Silicate, Sodium Citrate, Silica, Divinyldimethicone/Dimethicone Copolymer, Palmitic Acid, Ascorbyl Glucoside, Ammonium Polyacryldimethyltauramide/Ammonium Polyacryloyldimethyl Taurate, Xanthan Gum, Pisum Sativum Extract/Pea Extract, Acrylates Copolymer, Cetyl Alcohol, Butylene Glycol, Retinyl Palmitate, Imidazolidinyl Urea, Methylparaben, Phenoxyethanol, Butylparaben, Linalool, Limonene, Parfum/Fragrance, Potassium Sorbate, Chlorphenesin.

### Production Example 72. Face, Body, Hand Cream

Ingredients

Water, Glycerin, Mineral Oil, Alcohol Denat., Stearic Acid, Petrolatum, Cetyl Alcohol, Glyceryl Stearate, Hydrogenated Coco-Glycerides, Stearyl Alcohol, Dimethicone, Phenoxyethanol, Tocopheryl Acetate, Fragrance, Carbomer, Sodium Hydroxide, Methylparaben.

### Production Example 73. Body Cream

Ingredients

Water, Mineral Oil, Petrolatum, Glycerin, Microcrystalline Wax, Lanolin Alcohol, Paraffin, Panthenol, Magnesium Sulfate, Decyl Oleate, Octyldodecanol, Aluminum Stearates, Fragrance, Citric Acid, Magnesium Stearate, Methylchloroisothiazolinone, Methylisothiazolinone.

### Production Example 74. Antioxidant Gel-Cream Face Super Moisturizer

Ingredients

Aqua/Water, Glycerin, Dimethicone, Cetearyl, Ethylhexanoate, Alcohol Denat., Aloe Barbadensis Leaf Juice, Hydroxyethylpiperazine Ethane Sulfonic Acid, Ammonium Polyacryloyldimethyl Taurate, Phenoxyethanol, Caprylyl Glycol, Isopropyl Myristate, Tocopherol, Ascorbyl Glucoside, Dimethiconol, Bisabolol, Parfum/Fragrance, Sodium Hyaluronate, Xanthan Gum, Disodium Edta, T-Butyl Alcohol, Lycium Barbarum Fruit Extract, Sodium Hydroxide, Limonene, Farnesol, Sodium Benzoate, Linalool, Lactic Acid, Ceteareth-25, Citronellol, Potassium Sorbate, Geraniol, Cetyl Alcohol, Benzyl Alcohol, Ceramide Np, Behenic Acid, Punica Granatum Extract, Cholesterol, Ceramide Ns, Ethylhexylglycerin, Ci 42090/Blue 1, Ceramide Ap, Ceramide Eop, Ceramide Eos, Caprooyl Phytosphingosine, Caprooyl Sphingosine, Ascorbyl Palmitate

### Production Example 75. Brightening Moisturizing Cream

Ingredients

Water, cetyl alcohol, olea europaea (olive)fruit oil, glycerin, caprylic/capric triglyceride, tapiocastarch, prunus armeniaca (apricot) kernel oil, bellisperennis (daisy) flower extract, leucojum aestivum bulbextract, pearl powder, citrus limon (lemon) peel oil,emblica ocinalis fruit powder, simmondsia chinensis(jojoba) seed oil, helianthus annuus (sunflower) seed oil, vitis vinifera (grape) seed oil, melissa ocinalis leafextract, carrageenan, zea mays (corn) starch, candelilla/jojoba/rice bran polyglyceryl-3 esters, tocopherol, xanthan gum, sucrose, glyceryl stearate, sodium stearoyl lactylate, cetearyl alcohol, alcohol denat., phenethyl alcohol, sodium PCA, fragrance (natural fragrance), phenoxyethanol, citral, limonene, linalool.

### Production Example 76. Age Perfect Night Cream

Ingredients

Aqua/Water/Eau, Dimethicone, Glycerin, Paraffinum Liquidum/Mineral Oil/Huile Minerale, Octyldodecanol, Myristyl Myristate, Butyrospermum Parkii Butter/Shea Butter, Stearic Acid, Palmitic Acid, Ammonium Polyacryldimethyltauramide/Ammonium Polyacryloyldimethyl Taurate, Peg-100 Stearate, Cera Alba/Beeswax/Cire Dabeille, Glyceryl Stearate, Ethylhexyl Palmitate, Peg-20 Stearate, Hydrolyzed Soy Protein, Phenoxyethanol, Parfum/Fragrance, Capryloyl Salicylic Acid, Caprylyl Glycol, Acrylates Copolymer, Stearyl Alcohol, Cetyl Alcohol, Sodium Hydroxide, Jasminum Officinale Extract/Jasmine Flower Extract, Linalool, Limonene, Benzyl Alcohol, Geraniol, Citronellol, Eugenol, Coumarin, Citral, Alpha-Isomethyl Ionone

### Production Example 77. Hand Cream Fragrance Free

Ingredients

Water, Glycerin, Cetearyl Alcohol, Stearic Acid, Sodium Cetearyl Sulfate, Methylparaben, Propylparaben, Dilauryl Thiodipropionate, Sodium Sulfate

### Production Example 78. Hydro Boost Eye Gel-Cream

Ingredients

Water, Dimethicone, Glycerin, Cetearyl Olivate, Polyacrylamide, Sorbitan Olivate, Phenoxyethanol, Dimethicone/Vinyl Dimethicone Crosspolymer, Synthetic Beeswax, C13-14 Isoparafin, Dimethiconol, Carbomer, Dimethicone Crosspolymer, Chlorphenesin, Laureth-7, Sodium Hyaluronate, Ethylhexylglycerin, C12-14 Pareth-12, Sodium Hydroxide

### Production Example 79. Eye Firming Cream, Alpha-Hydroxy Acid

Ingredients

Water, cetyl alcohol, C12-15 alkyl benzoate, stearic acid, glycerin, glyceryl stearate, PEG-100 stearate, ethylhexyl palmitate, dimethicone, tocopheryl acetate, silica, glycolic acid, sodium hydroxide, xanthan gum, diazolidinyl urea, methylparaben, panthenol, bisabolol, melibiose, propylparaben, tetrasodium EDTA, ascorbic acid/orange/citrus limon/citrus aurantifolia polypeptides, retinyl palmitate, mica

### Production Example 80. Hand Cream, Honey and Grapeseed

Ingredients

Water, vitis vinifera (grape) seed oil, beeswax, glycerin, simmondsia chinensis (jojoba) butter, stearic acid, theobroma grandiflorum seed butter, macadamia ternifoliaseed oil, honey, helianthus annuus (sunflower) seed oil, rosmarinus o cinalis (rosemary) leaf extract, beta-carotene, tocopherol, xanthan gum, sodium borate, sucrose stearate, glucose, glycine soja (soybean) oil, sodium chloride, lactoperoxidase, glucose oxidase, canola oil (huile de colza), fragrance, potassium iodide, potassium thiocyanate, butylphenyl methylpropional, coumarin, hydroxyisohexyl3-cyclohexene carboxaldehyde.

### Production Example 81. Skin Firming and Toning Gel-cream

Ingredients

Water, Alcohol Denat., Glycerin, Dimethicone, Carnitine, Nelumbo Nucifera Leaf Extract, Ubiquinone, Glyceryl Glucoside, Chondrus Crispus Extract, Butylene Glycol, Acrylates/C10-30 Alkyl Acrylate Crosspolymer, Sodium Polyacrylate, Fragrance, Sodium Hydroxide, Phenoxyethanol, Blue 1.

### Production Example 82. Anti-wrinkle+Firming Eye Cream

Ingredients

Aqua/Water, Dimethicone, Glycerin, C12-15 Alkyl Benzoate, Shorea Robusta Butter/Shorea Robusta Seed Butter, Stearic Acid, Palmitic Acid, Peg-100 Stearate, Glyceryl Stearate, Peg-20 Stearate, Stearyl Alcohol, Paraffin, Glycine Soja Protein/Soybean Protein, Triethanolamine, Caffeine, Isohexadecane, Phenethyl Alcohol, Hydrolyzed Soy Protein, Acetyl Trifluoromethylphenyl Valylglycine, Faex Extract/Yeast Extract, Polysorbate 80, Acrylamide/Sodium Acryloyldimethyltaurate Copolymer, Acrylates Copolymer, Cetyl Alcohol, Retinyl Palmitate, Potassium Sorbate, Methylparaben, Sodium Benzoate, Chlorhexidine Digluconate, Ethylparaben, Phenoxyethanol

### Production Example 83. Firming Body Cream

Ingredients

Water, mineral oil, butylene glycol, glycerin, dimethicone, cetyl alcohol, cetyl ethylhexanoate, xylitol, microcrystalline wax, peg-60 glyceryl isostearate, peg-5 glyceryl stearate, phenoxyethanol, silica, fragrane, carbomer, paraffin, potassium hydroxide, prunus armeniaca (apricot) juice, alcohol, trisodium edta, linalool, butylphenyl methylpropional, sodium metaphosphate, agar, oryzanol, limonene, hexyl cinnamal, geraniol, citronellol, citrus unshiu peel extract, cinnamyl alcohol, crataegus monogyna flower extract, sodium hyaluronate, iron oxides (CI 77491), houttuynia cordata extract, bht, tocopherol

### Production Example 84. Cell Renewal Night Cream

Ingredients

Aqua/Water, Paraffinum Liquidum/Mineral Oil, Glycerin, Dimethicone, Squalane, Glyceryl Stearate, Cetyl Alcohol, Peg-40 Stearate, Cera Alba/Beeswax, Sorbitan Tristearate, Mel/Honey, Stearyl Alcohol, Cera Microcristallina/Microcrystalline Wax, Paraffin, Calcium Pantothenate, Dimethyl Isosorbide, Neohesperidin Dihydrochalcone, Isohexadecane, Sodium Cocoyl Glutamate, Myristyl Alcohol, Vigna Aconitifolia/Vigna Aconitifolia Seed Extract, Disodium Edta, Hydrolyzed Cicer Seed Extract, Hydroxyapatite, Capryloyl Salicylic Acid, Polysorbate 80, Acrylamide/Sodium Acryloyldimethyltaurate Copolymer, Acrylonitrile/Methyl Methacrylate/Vinylidene Chloride Copolymer, Octyldodecanol, Oryzanol, Tocopheryl Acetate, Pentaerythrityl Tetra-Di-T-Butyl Hydroxyhydrocinnamate, Phenoxyethanol Chlorphenesin, Ci 14700/Red 4, Ci 19140/Yellow 5, Linalool, Geraniol, Alpha-Isomethyl Ionone, Limonene, Hydroxycitronellal, Citronellol Hexyl Cinnamal, Benzyl Alcohol, Benzyl Salicylate, Parfum/Fragrance

### Production Example 85. Sensitive Night Cream

Ingredients

Water, helianthus annuus (sunflower) seed oil, glycerin, cetyl alcohol, CI 77947 (zinc oxide), zea mays (corn) starch, gossypium herbaceum (cotton) flower extract, oryza sativa (rice) extract, kaolin, aloe barbadensis leaf juice, sucrose, butyrospermum parkii (shea) butter, artemisia umbelliformis extract, jojoba esters, beta vulgaris (beet, extrait de racine de betterave) root extract, lecithin, saccharum officinarum (sugar cane, extrait de canne a sucre) extract, hydrolyzed jojoba esters, fructose, euphrasia officinalis extract, citric acid, xanthan gum, glucose, potassium sorbate, alcohol denat., sucrose polystearate, inositol, trehalose, sucrose stearate, benzoic acid, phenoxyethanol, sodium stearoyl lactylate, glyceryl laurate, sodium benzoate, sodium PCA.

### Production Example 86. Skin Nourishment Night Cream

Ingredients

Water, helianthus annuus (sunflower) seed oil, glycerin, cetylalcohol, stearic acid, sucrose stearate, lonicera japonica (honeysuckle) flowerextract, lonicera caprifolium (honeysuckle) flower extract, calendula o cinalisflower extract, hydrastis canadensis (goldenseal) extract, hamamelis virginiana(witch hazel) leaf extract, butyrospermum parkii (shea) butter, royal jelly, sucrose polystearate, hydrolyzed jojoba esters, xanthan gum, kaolin, lecithin, tocopherol, sodium phytate, citric acid, glycine soja (soybean) oil, jojoba esters, alcohol, fragrance (natural fragrance), phenoxyethanol, benzyl salicylate, limonene, linalool.

### Production Example 87. Age Defying Classic Night Cream

Ingredients

Water, Glycerin, PPG-15 Stearyl Ether, Stearyl Alcohol, Salicylic Acid, Cetyl Alcohol, Tocopheryl Acetate, Aloe Barbadensis Leaf Extract, Polyacrylamide, Dimethicone, C13-14 Isoparaffin, Steareth-21, Cyclopentasiloxane, Laureth-7, Fragrance, Dimethiconol, Steareth-2, Disodium EDTA, Yellow 5, Red 40

### Production Example 88. Essentially Enrich Body Cream

Ingredients

Water, Mineral Oil, Glycerin, Cetearyl Alcohol, Glyceryl Stearate, Glyceryl Stearate SE, Butyrospermum Parkii (Shea) Butter, Prunus Amydalus Dulcis (Sweet Almond) Oil, Hydrogenared Cocoglycerides, Dimethicone, Sodium Cetearyl Sulfate, Glyceryl Glucoside, Fragrance, Carbomer, Sodium Hydroxide, Phenoxyethanol, Methylparaben

### Production Example 89. Renewal Night Cream, Firming Night Cream

Ingredients

Water, glycerin, helianthus annuus (sunflower) seed oil, caprylic/capric triglyceride, decyl cocoate, coco-caprylate, decyl oleate, cetyl alcohol, stearic acid, beeswax, glyceryl stearate citrate, fragrance (natural fragrance), stearyl alcohol, hibiscus sabdariffa flower extract, pyrus malus (apple) fruit extract, citrullus lanatus (watermelon) fruit extract, lens esculenta fruit extract, scutellaria alpina flower/leaf/stem extract, adansonia digitata fruit extract, trifolium pratense (clover) leaf extract, plankton extract, beta vulgaris (beet) root extract, crambe abyssinica seed oil, phragmites communis extract, poria cocos extract, ribose, tocopherol, arginine, dipotassium glycyrrhizate, sclerotium gum, linoleic acid, glycine soja (soybean) oil, p-anisic acid, zea mays (corn) starch, coco-glucoside, hydrolyzed corn starch, magnesium aluminum silicate, xanthan gum, sodium stearoyl lactylate, cetearyl alcohol, sodium lactate, sodium PCA, citric acid, sodium citrate, lactic acid, linolenic acid, oleic acid, palmitic acid, sodium benzoate, potassium sorbate, phenoxyethanol, eugenol, limonene, linalool'

### Production Example 90. Hydra-Nutrition Honey Day Cream

Ingredients

Aqua/Water, Paraffinum Liquidum/Mineral Oil, Glycerin, Isocetyl Stearate, Hydrogenated Polyisobutene, Cyclohexasiloxane, Butyrospermum Parkii Butter/Shea Butter, Cera Alba/Beeswax, Cetyl Alcohol, Glyceryl Stearate, Peg-100 Stearate, Mel/Honey, Hydroxyethylpiperazine Ethane Sulfonic Acid, Prunus Armeniaca Kernel Oil/Apricot Kernel Oil, Zea Mays Oil/Corn Oil, Mel Extract/Honey Extract, Cera Microcristallina/Microcrystalline Wax, Paraffin, Royal Jelly Extract, Isobutane, Sorbitan Oleate, Sorbitan Tristearate, Calcium Pantetheine Sulfonate, Dimethicone, Copernicia Cerifera Cera/Carnauba Wax, Isohexadecane, Sodium Hydroxide, Ascorbyl Glucoside, Disodium Edta, Propylene Glycol, Caprylyl Glycol, Citric Acid, Passiflora Edulis Seed Oil, Xanthan Gum, Polysorbate 80, Acrylamide/Sodium Acryloyldimethyltaurate Copolymer, Acrylonitrile/Methyl Methacrylate/Vinylidene Chloride Copolymer, Oryza Sativa Bran Oil/Rice Bran Oil, Tocopherol, Ascorbyl Palmitate, Ci 15985/Yellow 6, Ci 19140/Yellow 5, Linalool, Geraniol, Eugenol, Coumarin, Limonene, Citronellol, Benzyl Alcohol, Parfum/Fragrance.

### Production Example 91. Refreshing Gel-Cream for Dry Skin

Ingredients

Aqua/Water, Glycerin, Alcohol Denat., Dimethicone, Isononyl Isononanoate, Ascorbyl Glucoside, Biosaccharide Gum-1, Caprylyl Glycol, Carbomer, Chlorphenesin, Ci 14700/Red 4, Dimethiconol, Menthoxypropanediol, Nymphaea Alba Extract/Nymphaea Alba Flower Extract, Panthenol, Phenoxyethanol, Pyrus Malus Water/Apple Fruit Water, Sodium Hydroxide, Sodium Palmitoyl Proline, Tetrasodium Edta, Tocopheryl Acetate, Xanthan Gum, Parfum/Fragrance.

### Production Example 92. Water Rose 24 H Moisture Cream

Ingredients

Aqua/water, glycerin, dimethicone, cetearyl, ethylhexanoate, alcohol denat, rosa damascena flower water, ammonium polyacryloyldimenthyl, taurate, hydroxyethylpiperazine ethane sulfonic acid, phenoxyethanol, caprylyl glycol, isopropyl myristate, ascorbyl glucoside, parfum/fragrance, dimethiconol, propanediol, sodium hyaluronate, xanthan gum, disodium edta, arginine, sodium hydroxide, citric acid, salicyclic acid.

### Production Example 93. Soothing Face Moisturizer with Rose Water

Ingredients

Aqua/Water, Glycerin, Butyrospermum Parkii Butter/Shea Butter, Caprylic/Capric Triglyceride, Cera Alba/Beeswax, Glyceryl Stearate Se, Polyglyceryl-3 Methylglucose Distearate, Alcohol Denat., Zea Mays Starch/Corn Starch, Stearyl Alcohol, Rosa Damascena Flower Water, Dicaprylyl Ether, Stearic Acid, Carbomer*, Glycine Soja Oil/Soybean Oil, Sodium Hydroxide*, Sodium Phytate, Arginine, Myristic Acid, Palmitic Acid, Prunus Armeniaca Kernel Oil/Apricot Kernel Oil, Propanediol, Caprylyl Glycol*, Citric Acid, Xanthan Gum, Tocopherol, Salicylic Acid*, Linalool*, Citronellol*, Parfum/Fragrance*

### Production Example 94. Skin Nourishment Hydrating Gel Cream

Ingredients

Water, dicaprylyl ether, sodium PCA, hydrolyzed quinoa, rice amino acids, royal jelly, lonicera japonica (honeysuckle) flower extract, lonicera caprifolium (honeysuckle) flower extract, chondrus crispus extract, tocopherol, cellulose gum, ceratonia siliqua gum, sclerotium gum, sucrose, glycerin, lysolecithin, pullulan, xanthan gum, sorbitan oleate decylglucoside crosspolymer, lactic acid, fragrance (natural fragrance), sodium benzoate, potassium sorbate, linalool

### Production Example 95. Anti-Aging Hydra Firming Cream

Ingredients

Water, Cyclopentasiloxane, Glycerin, Niacinamide, Pentylene Glycol, Ethylene/Acrylic Acid Copolymer, Dimethicone, Hexamidine Diisethionate, Palmitoyl Pentapeptide-4, Palmitoyl Dipeptide-7, Panthenol, Tocopheryl Acetate, PEG-10 Dimethicone, Dimethicone Crosspolymer, PEG-10 Dimethicone Crosspolymer, Stearyl Dimethicone, Bis-PEG/PPG-14/14 Dimethicone, Magnesium Sulfate, Benzyl Alcohol, Cetyl Ricinoleate, Methylparaben, Ethylparaben

### Production Example 96. Refreshing Gel-Cream for Normal/Combo Skin

Ingredients

Aqua/Water, Glycerin, Alcohol Denat., Dimethicone, Isononyl Isononanoate, Carbomer, Dimethiconol, Chlorphenesin, Caprylyl Glycol, Sodium Hydroxide, Vitis Vinfera Extract/Grapefruit Extract, Xanthan Gum, Tetrasodium Edta, Tocopheryl Acetate, Ascorbyl Glucoside, Menthoxypropanediol, Magnesium Gluconate, Sodium Citrate, Benzyl Salicylate, Linalool, Calcium Gluconate, Biosaccharide Gum-1, Hydroxyisohexyl 3-Cyclohexene Carboxaldehyde, Butylphenyl Methylpropional, Ci 19140/Yellow 5, Ci 42090/Blue 1, Parfum/Fragrance, Phenoxyethanol.

### Production Example 97. Face Moisturizer with Aloe, for Dry Skin

Ingredients

Aqua/Water, Glycerin, Aloe Barbadensis Leaf Juice, Dicaprylyl Ether, Alcohol Denat., Caprylic/Capric Triglyceride, Zea Mays Starch/Corn Starch, Polyglyceryl-3 Methylglucose Distearate, Stearyl Alcohol, Glyceryl Stearate Se, Carbomer*, Salicylic Acid*, Caprylyl Glycol*, Tocopherol, Parfum/Fragrance*, Sodium Hydroxide*, Xanthan Gum, Sodium Phytate, Glycine Soja Oil/Soybean Oil, Citric Acid, Potassium Sorbate*, Sodium Benzoate*

### Production Example 98. Dry Skin Relief Overnight Face Cream

Ingredients

Water (Aqua), Glycerin, Cyclopentasiloxane, Cetyl Alcohol, Caprylic/Capric Triglyceride, Cetyl Palmitate, Dipropylene Glycol, Butyrospermum Parkii (Shea) Butter, Dimethicone, Sodium Acrylate/Sodium Acryloyldimethyl Taurate Copolymer, Phenoxyethanol, Ceteth-20, Glyceryl Stearate, Caprylyl Glycol, Petrolatum, Carbomer, Disodium EDTA, Hydroxystearic Acid.

### Production Example 99. Hydrating Hand Gel Cream with Hyaluronic Acid

Ingredients

Water, Glycerin, Isopropyl Palmitate, Cetyl Alcohol, Dimethicone, Petrolatum, Phenoxyethanol, Sodium Polyacrylate, Cetearyl Olivate, Caprylyl Glycol, Ethylhexylglycerin, Sorbitan Olivate, Fragrance, Carbomer, Sodium Hyaluronate, Sodium Hydroxide, Blue 1

### Production Example 100. Hemp Hand Cream with Hemp Seed Oil for Dry Skin

Ingredients

Water, glycerin, helianthus annuus (sunflower) seed oil, cetyl alcohol, cocos nucifera (coconut) oil, adansonia digitate oil, caprylic/capric triglyceride, teobroma grandiflorum seed butter, fragrance*, butyrospermum parkii (shea) butter, cannabis sativa seed oil**, glyceryl stearate citrate, stearic acid, sucrose stearate, jojoba esters, helianthus annuus (sunflower) seed wax, acacia decurrens flower wax, xanthan gum, arginine, lactic acid, tocopherol, glycine soja (soybean) oil, polyglycerin-3, sodium stearoyl lactylate, magnesium aluminum silicate, sucrose polystearate, biosaccharide gum-1, glyceryl caprylate, sodium anisate, sodium levulinate, phenoxyethanol, citral, citronellol, eugenol, geraniol, limonene, linalool *natural fragrance **hemp seed oil.

### Production Example 101. Fragrance Free Facial Moisturizer

Ingredients

Ingredients: Water, Vinyl Dimethicone/Methicone Silsesquioxane Crosspolymer, Glycerin, Niacinamide*, Isohexadecane, Isopropyl Isostearate, Palmitoyl Pentapeptide-4**, Sodium Hyaluronate, Panthenol***, Ceratonia Siliqua (Carob) Fruit Extract, Dimethicone, Caprylyl Glycol Dimethiconol, Cetearyl Alcohol, Cetearyl Glucoside, Stearic Acid, Palmitic Acid, Stearyl Alcohol, Cetyl Alcohol, Behenyl Alcohol, Disodium Edta, Peg-100 Stearate, Sodium Polyacrylate Starch, 1, 2-Hexanediol, Phenoxyethanol.

### Production Example 102. Rapid Wrinkle Repair Night Moisturizer

Ingredients

Water, pentaerythrityl tetraethylhexanoate, dimethicone, glycerin, PPG-15 stearyl ether, cetearyl alcohol, stearyl alcohol, butylene glycol, ceteareth-20, isohexadecane, trisiloxane, dimethicone crosspolymer, phenoxyethanol, caprylyl glycol, ammonium acryloyldimethyltaurate/VP copolymer, polyacrylamide, polyethylene, fragrance, C13-14 isoparaffin, chlorphenesin, PTFE, hydrolyzed myrtus communis leaf extract, polysorbate 20, retinol, BHT, sodium hyaluronate, disodium EDTA, laureth-7, ascorbic acid, sodium hydroxide

### Production Example 103. Triple Age Repair Anti-Aging Night Face Moisturizer

Ingredients

Water, Glycerin, Dimethicone, Isononyl Isononanoate, Ethylhexyl Palmitate, Propylene Glycol, Steareth-2, Butyrospermum Parkii (Shea) Butter, Methyl Methacrylate Crosspolymer, Steareth-21, Hexylresorcinol, Ammonium Acryloyldimethyltaurate/VP Copolymer, Behenyl Alcohol, Dimethicone Crosspolymer, Caprylyl Glycol, Ascorbyl Glucoside, Sclerotium Gum, Xylitol, Xylitylglucoside, Anhydroxylitol, Disodium EDTA, Stearyl Alcohol, Arachidyl Alcohol, Cetyl Alcohol, Lignoceryl Alcohol, Sodium Hydroxide, Phenoxyethanol, Methylparaben, Propylparaben, Ethylparaben, Titanium Dioxide, Mica, Fragrance.

### Production Example 104. Calming Facial Moisturizer, Fragrance-Free

Ingredients

Water, Glycerin, Panthenol, Isohexadecane, Niacinamide*, Tapioca Starch, Isopropyl Isostearate, Stearyl Alcohol, Polyacrylamide, Aloe Barbadensis Leaf Juice, Cucumis Sativus (Cucumber) Fruit Extract, Camellia Sinensis Leaf Extract, Tocopheryl Acetate, Hydroxyacetophenone, C13-14 Isoparaffin, Laureth-7, Cetearyl Glucoside, Cetearyl Alcohol, Stearic Acid, Palmitic Acid, Cetyl Alcohol, Behenyl Alcohol, Disodium EDTA, PEG-100 Stearate, Titanium Dioxide, Polymethylsilsesquioxane, Phenoxyethanol, *Vitamin B3.

### Production Example 105. Triple Power Deep-Acting Moisturizer

Ingredients

Aqua/Water, Glycerin, Isohexadecane, Isopropyl Isostearate, Hydroxypropyl Tetrahydropyrantriol, Aluminum Starch Octenylsuccinate, Propylene Glycol Nylon-12, Octyldodecanol, Cetyl Alcohol, Ammonium Polyacryldimethyltauramide/Ammonium Polyacryloyldimethyl Taurate, Behenyl Alcohol, Dimethicone, C13-14 Isoparaffin, PEG-100 Stearate, Stearic Acid, Stearyl Alcohol, Arachidyl Alcohol, Triethanolamine, Cetearyl Alcohol, Cetearyl Glucoside, Sodium Hyaluronate, Palmitic Acid, Adenosine, Disodium EDTA, Disodium Stearoyl Glutamate, Hydrolyzed Hyaluronic Acid, Capryloyl Salicylic Aid, Laureth-7, Polyacrylamide, Polyethylene, Methylisothiazolinone, Methylparaben, Phenoxyethanol, CI 15985/Yellow 6, CI 19140/Yellow 5, CI 77891/Titanium Dioxide, Mica, Linalool, Isoeugenol, Limonene, Citronellol, Benzyl Alcohol, Parfum/Fragrance.

### Production Example 106. Triple Power Intensive Anti-Aging Day Cream Moisturizer

Ingredients

Water, Glycerin, Isohexadecane, Isopropyl Isostearate, Hydroxypropyl Tetrahydropyrantriol, Aluminum Starch Octenylsuccinate, Propylene Glycol, Nylon-12, Octyldodecanol, Cetyl Alcohol, Ammonium Polyacryldimethyltauramide/Ammonium Polyacryloyldimethyl Taurate, Behenyl Alcohol, Dimethicone, C13-14 Isoparaffin, Peg-100 Stearate, Stearic Acid, Stearyl Alcohol, Arachidyl Alcohol, Triethanolamine, Cetearyl Alcohol, Cetearyl Glucoside, Sodium Hyaluronate, Palmitic Acid, Adenosine, Disodium Edta, Disodium Stearoyl Glutamate, Hydrolyzed Hyaluronic Acid, Capryloyl Salicylic Acid, Laureth-7, Polyacrylamide, Polyethylene, Methylparaben, Phenoxyethanol, Ci 15985/Yellow 6, Ci 19140/Yellow 5, Ci 77891/Titanium Dioxide, Mica, Linalool, Isoeugenol, Limonene, Citronellol, Benzyl Alcohol, Fragrace.

### Production Example 107. Anti-Aging Eye Transforming Cream

Ingredients

Water, Glycerin, Petrolatum, Isopropyl Isostearate, Isohexadecane, Niacinamide, Stearyl Alcohol, Dimethicone, Cetyl Alcohol, Tocopheryl Acetate, Panthenol, Camellia Sinensis Leaf Extract, Allantoin, Aloe Barbadensis Leaf Extract, Cucumis Sativus (Cucumber) Fruit Extract, C13-14 Isoparaffin, Caprylyl Glycol, Dimethiconol, Cetearyl Glucoside, Cetearyl Alcohol, Laureth-7, Stearic Acid, Palmitic Acid, Behenyl Alcohol, Disodium EDTA, Polyacrylamide, Sodium Hydroxide, PEG-100 Stearate, Titanium Dioxide, Mica, 1,2-Hexanediol, Silica, Phenoxyethanol, Sodium Benzoate, Iron Oxides.

### Production Example 108. Eye Cream for Wrinkles, Puffy Eyes+Dark Circles

Ingredients

Water, Vinyl Dimethicone/Methicone Silsesquioxane Crosspolymer, Glycerin, Niacinamide, Isohexadecane, Isopropyl Isostearate, Stearyl Alcohol, Palmitoyl Pentapeptide-4, Dimethicone, Cetyl Alcohol, Behenyl Alcohol, Polyacrylamide, C13-14 Isoparaffin, Benzyl Alcohol, Ethylparaben, Peg-100 Stearate, Dimethiconol, Methylparaben, Propylparaben, Stearic Acid, Cetearyl Glucoside, Cetearyl Alcohol, Laureth-7, Disodium Edta, Titanium Dioxide, Iron oxides.

### Production Example 109. Brightening Gel Moisturizer Face Cream

Ingredients

Water, dimethicone, glycerin, mandelic acid, triethyl citrate, gluconolactone, acetyl glucosamine, hydroxyethyl acrylate/sodium acryloyldimethyl taurate copolymer, polyacrylate crosspolymer-6, butylene glycol, squalane, aluminum starch octenylsuccinate, sodium hydroxide, phenoxyethanol, dimethicone crosspolymer, cetearyl olivate, dimethiconol, sorbitan olivate, chlorphenesin, cetearyl alcohol, polysorbate 60, succinoglycan, coco-glucoside, disodium edta, fragrance, ethylhexylglycerin, silica, tin oxide, titanium dioxide, mica, red 40.

### Production Example 110. Eye Defense Eye Cream

Ingredients

Aqua/Water/Eau, Glycerin, Simmondsia Chinensis Seed Oil/Jojoba Seed Oil Propanediol, Pentaerythrityl Tetraethylhexanoate-Polyglyceryl, 2 Stearate, Phenoxyethanol, Carbomer, Caffeine, Myristyl Alcohol, Bisabolol, Methylparaben, Sodium Hydroxide, Xanthan Gum, Mica, Ethylparaben, Disodium Edta, Avena Sativa Kernel Extract/Oat Kernel Extract, Citric Acid, Ci 77891/Titanium Dioxide, Farnesol, Sodium Hyaluronate, Ci 16035/Red 40.

### Production Example 111. Dark Circle Repair Eye Cream

Ingredients

Water, Dimethicone, Pentaerythrityl Tetraethylhexanoate, Methyl Trimethicone, Dimethicone/Peg10/15 Crosspolymer, Butylene Glycol, C3045 Alkyl Methicone, Glycerin, Dicaprylyl Carbonate, C3045 Olefin, Trisiloxane, Peg9 Polydimethylsiloxyethyl Dimethicone, Silica, Zinc, Phenoxyethanol, Polyglyceryl4 Isostearate, Sodium Chloride, Cetyl Peg/Ppg-10/1 Dimethicone, Hexyl Laurate, Chlorphenesin, Disodium Edta, Ethylhexylglycerin, Peg/Ppg18/18 Dimethicone, Triethoxycaprylylsilane, Copper Powder, Mica, Titanium Dioxide, Iron Oxides.

### Production Example 112. Oil Intensive Body Lotion

Ingredients Water, Glycerin, Stearic Acid, Caprylic/Capric Triglyceride, Dimethicone, Glycol Stearate, Peg-100 Stearate, Petrolatum, Cyclopentasiloxane, Acrylates/C10-30 Alkyl Acrylate Crosspolymer, Glyceryl Stearate, Cetyl Alcohol, Fragrance, Stearamide Amp, Phenoxyethanol, Triethanolamine, Carbomer, Disodium Edta, Methylparaben, Propylparaben, Titanium Dioxide.

### Production Example 113. Triple Power Eye Treatment

Ingredients

Aqua/Water, Dimethicone, Glycerin, Silica, Acrylamide/Sodium Acryloyldimethyltaurate Copolymer, Prunus Armeniaca Kernel Oil/Apricot Kernel Oil, Triethanolamine, Dimethicone/Vinyl Dimethicone Crosspolymer, Dimethiconol, Caffeine, Isohexadecane, Adenosine, Disodium Edta, Propylene Glycol, Hydrolyzed Hyaluronic Acid, Hydroxyethylpiperazine Ethane Sulfonic Acid, Hydroxypropyl Tetrahydropyrantriol, Polysorbate 80, Acrylates/C10-30 Alkyl Acrylate Crosspolymer, Tocopheryl Acetate, Phenoxyethanol, Chlorphenesin, Ci 77891/Titanium Dioxide, Mica.

### Production Example 114. Eye Renewal Cream

Ingredients

Water, Cyclohexasiloxane, Glycerin, Alcohol Denat., Paraffinum Liquidum/Mineral Oil, Prunus Armeniaca Kernel Oil/Apricot Kernel Oil, Butyrospermum Parkii Butter/Shea Butter, Dimethicone, Stearic Acid, Palmitic Acid, Peg-100 Stearate, Cera Alba/Beeswax, Glyceryl Stearate, Cetearyl Alcohol, Silica, Rosa Canina Fruit Oil, Tin Oxide, C13-14 Isoparaffin, Peg-20 Stearate, Triethanolamine, Dimethiconol, Caffeine, Sodium Cocoyl Glutamate, Vigna Aconitifolia/Vigna Aconitifolia Seed Extract, Ammonium Polyacryldimethyltauramide/Ammonium Polyacryloyldimethyl Taurate, Disodium Edta, Propylene Glycol, Hydrolyzed Cicer Seed Extract, Hydroxypropyl Tetrahydropyrantriol, Capryloyl Salicylic Acid, Laureth-7, Lauroyl Lysine, Synthetic Fluorphlogopite, Oxothiazolidinecarboxylic Acid, Ethylhexyl Methoxycinnamate, Polyacrylamide, Polymethyl Methacrylate, Polyurethane-2, Octyldodecanol, Retinyl Palmitate, Tocopherol, Phenoxyethanol, Ci 16035/Red 40, Ci 19140/Yellow 5, Ci 77891/Titanium Dioxide, Sodium Dehydroacetate.

### Production Example 115. Tone Perfecting Cream Face Moisturizer

Ingredients

Water, Cyclopentasiloxane, Glycerin, Niacinamide*, Aluminum Starch Octenylsuccinate, Acetyl Glucosamine, Dimethicone, Dimethicone Crosspolymer, Polyethylene, Panthenol**, Tocopheryl Acetate***, Palmitoyl Pentapeptide-4\hat{ }, Camellia Sinensis Leaf Extract\hat{ }\hat{ }, Aloe Barbadensis Leaf Juice\hat{ }\hat{ }\hat{ }, Allantoin, Sodium Peg-7 Olive Oil Carboxylate, Acrylamide/Sodium Acryloyldimethyltaurate Copolymer, Dmdm Hydantoin, Laureth-10, Laureth-7, Dimethiconol, Sodium Ascorbyl Phosphate, Acrylates/C10-30 Alkyl Acrylate Crosspolymer, Disodium Edta, Peg-100 Stearate, lodopropynyl Butylcarbamate, C13-14 Isoparaffin, Fragrance, Titanium Dioxide, Yellow 5, Red 40.

### Production Example 116. Anti-Wrinkle Day Cream+Serum

Ingredients

Water/Eau, Cyclopentasiloxane, Glycerin, Aluminum Starch Octenylsuccinate, Niacinamide, Dimethicone, Dimethicone Crosspolymer, Polyethylene, Panthenol, Tocopheryl Acetate, Camellia Sinensis Leaf Extract, Dimethiconol, Polyacrylamide, C13-14 Isoparaffin, Laureth-7, Laureth-4, Benzophenone-4, Bht, Disodium Edta, Sodium Hydroxide, Dmdm Hydantoin, lodopropynyl Butylcarbamate, Fragrance/Parfum, Titanium Dioxide, Mica, Yellow 5, Red 40.

### Production Example 117. Active Hydrating Skin Cream

Ingredients

Water/Eau, Glycerin, Cetyl Alcohol, Petrolatum, Cyclopentasiloxane, Stearyl Alcohol, Isopropyl Palmitate, Dimethicone, Carbomer, Sodium Hydroxide, Stearic Acid, Peg-100 Stearate, Fragrance/Parfum, Peg/Ppg-18/18 Dimethicone, Titanium Dioxide, Edta, Dmdm Hydantoin, lodopropynyl Butylcarbamate, Mdm Hydantoin, Red 4.

### Production Example 118. Under Eye Anti-Wrinkle Cream

Ingredients

Water, Dimethicone, Propylene Glycol, Glycerin, Trisiloxane, Ammonium Acryloyldimethyltaurate/Vp Copolymer, Dimethicone Crosspolymer, C12-15 Alkyl Benzoate, Methyl Methacrylate Crosspolymer, Aluminium Starch Octenylsuccinate, Ascorbyl Glucoside, Caprylyl Glycol, Hexylresorcinol, Nylon-12, Polyacrylamide, Xanthan Gum, C13-14 Isoparaffin, Tocopheryl Acetate, Maltodextrin, Disodium Edta, Sodium Hydroxide, Laureth-7, Butylene Glycol, Peucedanum Graveolens (Dill) Extract, Rubus Fruticosus (Blackberry) Leaf Extract, Mica, Titanium Dioxide.

### Production Example 119. Brightening Eye Cream for Dark Circles Facial Moisturizer

Ingredients

Water, petrolatum, isopropyl isostearate, tapioca starch, isohexadecane, glycerin, niacinamide*, caffeine, stearyl alcohol, cetyl alcohol, magnesium ascorbyl phosphate**, citrus aurantium dulcis (orange) flower extract***, panthenol\hat{ }, tocopheryl acetate\hat{ }\hat{ }, palmitoyl pentapeptide-\hat{ }\hat{ }\hat{ }, camellia sinensis leaf extract, C13-14 isoparaffin, caprylyl glycol, cetearyl glucoside, cetearyl alcohol, laureth-7, stearic acid, palmitic acid, behenyl alcohol, disodium edta, polyacrylamide, sodium hydroxide, peg-100 stearate, titanium dioxide, mica, polymethylsilsesquioxane, 1,2-hexanediol, phenoxyethanol, fragrance. *Vitamin B3, **Vitamin C, ***Orange flower extract, \hat{ }Pro-vitamin B5, \hat{ }\hat{ }Vitamin E, \hat{ }\hat{ }\hat{ }Amino-peptide.

### Production Example 120. Fragrance Free Micro-Sculpting Cream

Ingredients

Water, Glycerin, Isohexadecane, Niacinamide, Isopropyl Isostearate, Aluminum Starch Octenylsuccinate, Nylon-12, Dimethicone, Panthenol, Tocopheryl Acetate, Palmitoyl Pentapeptide-4, Sodium Peg-7 Olive Oil Carboxylate, Sodium Hyaluronate, Caprylic/Capric Triglyceride, Ceratonia Siliqua (Carob) Fruit Extract, Stearyl Alcohol, Polyethylene, Cetyl Alcohol, Behenyl Alcohol, Sodium Acrylates Copolymer, Titanium Dioxide, Benzyl Alcohol, Methylparaben, Ethylparaben, Dimethiconol, Polyacrylamide, Peg-100 Stearate, Propylparaben, Cetearyl Glucoside, Cetearyl Alcohol C13-14 Isoparaffin, Disodium Edta, Citric Acid, Stearic Acid, Palmitic Acid, C12-13 Pareth-3, Laureth-7, Sodium Hydroxide, Mica.

### Production Example 121. Body Cream

Ingredients

Water, Petrolatum, Caprylic/Capric Triglyceride, Stearic Acid, Glycerin, Sodium Hydroxypropyl Starch Phosphate, Glycol Stearate, Peg-100 Stearate, Glyceryl Stearate, Cetyl Alcohol, Phenoxyethanol, Methylparaben, Propylparaben, Stearamide Amp, Acrylates/C10-30 Alkyl Acrylate Crosspolymer, Disodium Edta, Tocopheryl Acetate, Titanium Dioxide (Ci 77891).

### Production Example 122. Dark Spot Rescue Hand and Body Lotion

Ingredients

Water (Aqua), Glycerin, Stearic Acid, Caprylic/Capric Triglyceride, Dimethicone, Glycol Stearate, Peg-100 Stearate, Cyclopentasiloxane, Niacinamide, Petrolatum, Glyceryl Stearate, Phenoxyethanol, Cetyl Alcohol, Triethanolamine, Methylparaben, Acrylates/C10-30 Alkyl Acrylate Crosspolymer, Fragrance (Parfum), Carbomer, Propylparaben, Disodium Edta, Stearamide Amp, Hydroxystearic Acid, Titanium Dioxide (Ci 77891).

### Production Example 123. Deep Hydration Regenerating Cream Face Moisturizer

Ingredients

Water, Dimethicone, Glycerin, Niacinamide*, Dimethicone Crosspolymer, Polymethylsilsesquioxane, Acrylamide/Sodium Acryloyldimethyltaurate Copolymer, Panthenol**, Polyethylene, Palmitoyl Pentapeptide-4***, Camellia Sinensis Leaf Extract\hat{ }, Tocopheryl Acetate\hat{ }\hat{ }, Sodium Peg-7 Olive Oil Carboxylate, Allantoin, Dmdm Hydantoin, Dimethiconol, Acrylates/C10-30 Alkyl Acrylate Crosspolymer, Aminomethyl Propanol, Disodium Edta, Iodopropynyl Butylcarbamate, Laureth-4, Laureth-7, Peg-100 Stearate, C13-14 Isoparaffin, Fragrance, Red 40, Yellow 5, Titanium Dioxide.

### Production Example 124. Body Cream

Ingredients

Water, Petrolatum, Caprylic/Capric Triglyceride, Stearic Acid, Glycerin, Sodium Hydroxypropyl Starch Phosphate, Glycol Stearate, Peg-100 Stearate, Glyceryl Stearate, Cetyl Alcohol, Phenoxyethanol, Methylparaben, Propylparaben, Stearamide Amp, Acrylates/C10-30 Alkyl Acrylate Crosspolymer, Disodium Edta, Tocopheryl Acetate, Titanium Dioxide (Ci 77891).

### Production Example 125. Anti-Aging Eye Restoration Complex

Ingredients

Water, Cyclopentasiloxane, Glycerin, Niacinamide, Dimethicone, Polymethylsilsesquioxane, Isohexadecane, Polyethylene, Isopropyl Isostearate, Caffeine, Palmitoyl Pentapeptide-4, Palmitoyl Dipeptide-7, Carnosine, Panthenol, Tocopheryl Acetate, Dimethicone Crosspolymer, Stearyl Alcohol, PEG-100 Stearate, Cetearyl Glucoside, Cetearyl Alcohol, Cetyl Alcohol, Sodium Acrylate/Sodium Acryloyldimethyl Taurate Copolymer, Behenyl Alcohol, Caprylyl Glycol, 1,2-Hexanediol, Phenoxyethanol, Titanium Dioxide, Dimethiconol, Polysorbate 80, Sodium Benzoate, Stearic Acid, Palmitic Acid, Disodium EDTA, Citric Acid.

### Production Example 126. Skin Empowering Cream

Ingredients

Water (Aqua), Glycerin, Dipropylene Glycol, Pentaerythrityl Tetraethylhexanoate, Diphenylsiloxy Phenyl Trimethicone, Mineral Oil (Paraffinum Liquidum), Dimethicone, Alcohol Denat, Behenyl Alcohol, Petrolatum, Carboxydecyl Trisiloxane, Batyl Alcohol, PEG-20 Stearate, Erythritol, PEG/PPG-14/7 DIMETHYL ETHER, Carnosine, Phytosteryl Macadamiate, Phenoxyethanol, Tocopheryl Acetate, Titanium Dioxide (CI 77891), Euphorbia Cerifera (Candelilla) Wax (Candelilla Cera), Glyceryl Stearate, Sorbitan Tristearate, Fragrance (Parfum), Carbomer, Succinoglycan, Polyquaterium-51, Talc, Potassium Hydroxide, Trisodium EDTA, Sodium Metaphosphate, Silica, Alumina, Limonene, Linalool, Benzyl Benzoate, Iron Oxides (CI 77492), Butylphenyl Methylpropional, Fagus Sylvatica Bud Extract, Tocopherol.

### Production Example 127. Firming Night Cream

Ingredients

Water/Eau, Glycerin, Cetyl Palmitate, Cetyl Alcohol, Cyclopentasiloxane, Stearyl Alcohol, Isopropyl Palmitate, Dimethicone, Carbomer, Stearic Acid, Peg-100 Stearate, Fragrance/Parfum, Sodium Hydroxide, Peg/Ppg-18/18 Dimethicone, Dmdm Hydantoin, Titanium Dioxide, Edta, lodopropynyl Butylcarbamate, Mdm Hydantoin, Red 4.

### Production Example 128. Night Eye Cream

Ingredients

Water, Dimethicone, Glycerin, Dimethicone Crosspolymer, Retinol, Retinyl Propionate, Niacinamide*, Tocopheryl Acetate**, Polyacrylamide, C13-14 Isoparaffin, Titanium Dioxide, DMDM Hydantoin, Polysorbate 20, Laureth-4, Acrylates/C10-30 Alkyl Acrylate Crosspolymer, Laureth-7, Dimethiconol, Aminomethyl Propanol, Disodium EDTA, lodopropynyl Butylcarbamate, Capryl/Caprylic Triglyceride, *Vitamin B3, ** Vitamin E.

### Production Example 129. Fragrance-Free Night Recovery Cream Moisturizer

Ingredients

Water, Dimethicone, Glycerin, Niacinamide, Dimethicone Crosspolymer, Polymethylsilsesquioxane, Polyacrylamide, Polyethylene, Panthenol, Tocopheryl Acetate, Palmitoyl Pentapeptide-4, Allantoin, Camellia Sinensis Leaf Extract, Sodium Peg-7 Olive Oil Carboxylate, C13-14 Isoparaffin, Titanium Dioxide, Dmdm Hydantoin, Laureth-4, Dimethiconol, Laureth-7, Acrylates/C10-30 Alkyl Acrylate Crosspolymer, Aminomethyl Propanol, Disodium Edta, lodopropynyl Butylcarbamate.

### Production Example 130. Fragrance Free Face Moisturizer

Ingredients

Aqua/Water/Eau, Squalane, Cyclohexasiloxane, Glycerin, Cetyl Alcohol, Glyceryl Stearate, Peg-40 Stearate, Myristyl Myristate, Pentaerythrityl Tetraethylhexanoate, Octyldodecanol, Sorbitan Tristearate, Butylene Glycol, Phenoxyethanol, Acrylamide/Sodium Acryloyldimethyltaurate Copolymer, Methyldihydrojasmonate, Synthetic Fluorphlogopite, Ci 77891/Titanium Dioxide, Isohexadecane, Capryloyl Salicylic Acid, Acrylonitrile/Methyl Methacrylate/Vinylidene Chloride Copolymer, Mannitol, Ascorbyl Glucoside, Chlorphenesin, Mica, Polysorbate 80, Paeonia Albiflora Root Extract, Disodium Edta, Ci 77163/Bismuth Oxychloride, Ci 77491/Iron Oxides, Hydrolyzed Cicer Seed Extract, Sorbitan Oleate, Isobutane, Sodium Hydroxide, Iris Florentina Root Extract, Vigna Aconitifolia Seed Extract, Cyclodextrin, Sodium Cocoyl Glutamate, Faex Extract/Yeast Extract/Extrait De Levure, Tin Oxide, Disodium Succinate, Bht.

### Production Example 131. Peptide-C Facial Moisturizer

Ingredients

Aqua/Water/Eau, Glycerin, Dimethicone, Isohexadecane, Alcohol Denat., Silica, Hydroxyethylpiperazine Ethane Sulfonic Acid, Propanediol, Synthetic Wax, Peg-10 Dimethicone, Eperua Falcata Bark Extract, Peg-30 Dipolyhydroxystearate, Stearyl Alcohol, Sorbitan Oleate, Dimethicone/Peg-10/15 Crosspolymer, Dimethicone/Polyglycerin-3 Crosspolymer, Ceteareth-6, Sodium Acrylates Copolymer, Sodium Citrate, Sodium Hydroxide, Sodium Phytate, Cyclodextrin, Adenosine, Ascorbyl Glucoside, Nylon-12, Isopropyl Alcohol, Propylene Carbonate, Dipropylene Glycol, Caprylic/Capric Triglyceride, Caprylyl Glycol, Acetyl Tetrapeptide-9, Synthetic Fluorphlogopite, Disteardimonium Hectorite, Dextrin, Pisum Sativum Extract/Pea Extract, Tocopherol, Phenoxyethanol, Ci 15985/Yellow 6, Ci 77891/Titanium Dioxide, Parfum/Fragrance.

### Production Example 132. Anti-Wrinkle Cream

Ingredients

Water, Lactic Acid, Butylene Glycol, Glycerin, Cetyl Alcohol, Isopropyl Palmitate, Steareth-21, Dimethicone, Mineral Oil, Ethylhexyl Methoxycinnamate, Stearyl Dimethicone, Steareth-2, Hydroxyethyl Acrylate/Sodium Acryloyldimethyl Taurate Copolymer, Isohexadecane, Polysorbate 60, Magnesium Aluminum Silicate, Stearic Acid, Borago Officinalis Seed Oil, Fragrance, Methylparaben Hydroxyethylcellulose, Titanium Dioxide, Xanthan Gum, Dimethiconol, Disodium Edta, Benzophenone-3, Propylparaben, Tocopheryl Acetate, Hydrolyzed Collagen, Phospholipids.

### Production Example 133. Night Firming Facial Moisturizer

Ingredients

Water, Glycerin, Niacinamide, Isohexadecane, Dimethicone, Isopropyl Isostearate, Stearyl Alcohol, Polyacrylamide, Tocopheryl Acetate, Camellia Sinensis Leaf Extract, Sodium Ascorbyl Phosphate, Panthenol, Cetyl Alcohol, C13-14 Isoparaffin, Behenyl Alcohol, Polymethylsilsesquioxane, Titanium Dioxide, Caprylyl Glycol, 1, 2-Hexanediol, Phenoxyethanol, Dimethiconol, Fragrance, Laureth-7, Sodium Benzoate, Peg-100 Stearate, Cetearyl Glucoside, Cetearyl Alcohol, Disodium Edta, Stearic Acid, Palmitic Acid.

### Production Example 134. Micro-Sculpting Cream Face Moisturizer

Ingredients

Water, Glycerin, Isohexadecane, Niacinamide, Isopropyl Isostearate, Aluminum Starch Octenylsuccinate, Nylon-12, Dimethicone, Panthenol, Tocopheryl Acetate, Palmitoyl Pentapeptide-4, Sodium Peg-7 Olive Oil Carboxylate, Sodium Hyaluronate, Caprylic/Capric Triglyceride, Ceratonia Siliqua (Carob) Fruit Extract, Stearyl Alcohol, Polyethylene, Cetyl Alcohol, Behenyl Alcohol, Sodium Acrylates Copolymer, Titanium Dioxide, Benzyl Alcohol, Methylparaben, Ethylparaben, Dimethiconol, Polyacrylamide, Fragrance, Peg-100 Stearate, Propylparaben, Cetearyl Glucoside, Cetearyl Alcohol, C13-14 Isoparaffin, Disodium Edta, Citric Acid, Stearic Acid, Palmitic Acid, C12-13 Pareth-3, Laureth-7, Sodium Hydroxide, Mica.

### Production Example 135. Fragrance-Free Micro-sculpting Cream Face Moisturizer

Ingredients

Water, Glycerin, Isohexadecane, Niacinamide, Isopropyl Isostearate, Aluminum Starch Octenylsuccinate, Nylon-12, Dimethicone, Panthenol, Tocopheryl Acetate, Palmitoyl Pentapeptide-4, Sodium PEG-7 Olive Oil Carboxylate, Sodium Hyaluronate, Caprylic/Capric Triglyceride, Ceratonia Siliqua (Carob) Fruit Extract, Stearyl Alcohol, Polyethylene, Cetyl Alcohol, Behenyl Alcohol, Sodium Acrylates Copolymer, Titanium Dioxide, Benzyl Alcohol, Methylparaben, Ethylparaben, Dimethiconol, Polyacrylamide, PEG-100 Stearate, Propylparaben, Cetearyl Glucoside, Cetearyl Alcohol, C13-14 Isoparaffin, Disodium EDTA, Citric Acid, Stearic Acid, Palmitic Acid, C12-13 Pareth-3, Laureth-7, Sodium Hydroxide, Mica.

### Production Example 136. Recovery Night Cream Face Moisturizer

Ingredients

Water, Dimethicone, Glycerin, Niacinamide, Dimethicone Crosspolymer, Polymethylsilsesquioxane, Polyacrylamide, Polyethylene, Panthenol, Tocopheryl Acetate, Palmitoyl Pentapeptide-4, Allantoin, Camellia Sinensis Leaf Extract, Sodium Peg-7 Olive Oil Carboxylate, C13-14 Isoparaffin, Titanium Dioxide, Dmdm Hydantoin, Laureth-4, Dimethiconol, Laureth-7, Acrylates/C10-30 Alkyl Acrylate Crosspolymer, Aminomethyl Propanol, Disodium Edta, lodopropynyl Butylcarbamate.

### Production Example 137. Ultra-Lift Anti-Wrinkle Firming Eye Cream

Ingredients

Aqua/Water, Butylene Glycol, Glycerin, Dimethicone, Isopropyl Palmitate, Cetearyl Alcohol, Isopropyl Lauroyl Sarcosinate, Alumina, Ammonium Polyacryldimethyltauramide/Ammonium Polyacryloyldimethyl Taurate, Ascorbyl Glucoside, Butyrospermum Parkii Butter/Shea Butter, Caffeine, Capryloyl Salicylic Acid, Chlorhexidine Digluconate, Chlorphenesin, Ci 77891/Titanium Dioxide, Dimethicone/Vinyl Dimethicone Crosspolymer, Dimethiconol, Disodium Edta, Glyceryl Stearate, Hydrolyzed Rice Protein, Isopropyl Titanium Triisostearate, Methylparaben, Mica, Palmitic Acid, Peg-100 Stearate, Peg/Ppg/Polybutylene Glycol-8/5/3 Glycerin, Phenoxyethanol, Retinyl Linoleate, Sodium Hydroxide, Sodium Polyacrylate, Stearic Acid, Zea Mays Starch/Corn Starch.

### Production Example 138. Anti-Aging Night Firming Cream

Ingredients

Water, Glycerin, Niacinamide, Isohexadecane, Dimethicone, Isopropyl Isostearate, Stearyl Alcohol, Polyacrylamide, Tocopheryl Acetate, Camellia Sinensis Leaf Extract, Sodium Ascorbyl Phosphate, Panthenol, Cetyl Alcohol, C13-14 Isoparaffin, Behenyl Alcohol, Polymethylsilsesquioxane, Titanium Dioxide, Caprylyl Glycol, 1,2-Hexanediol, Phenoxyethanol, Dimethiconol, Fragrance, Laureth-7, Sodium Benzoate, Peg-100 Stearate, Cetearyl Glucoside, Cetearyl Alcohol, Disodium Edta, Stearic Acid, Palmitic Acid.

### Production Example 139. Triple Power Anti-Aging Cream, Fragrance Free

Ingredients

Aqua/Water/Eau, Glycerin, Isohexadecane, Isopropyl Isostearate, Niacinamide, Aluminum Starch Octenylsuccinate, Nylon-12, Dimethicone, Cetyl Alcohol, Polyethylene, Stearyl Alcohol, Cetearyl Alcohol, Stearic Acid, Behenyl Alcohol, Phenoxyethanol, Synthetic Fluorphlogopite, Palmitic Acid, Polyacrylamide, Capryloyl Salicylic Acid, Sodium Acrylates Copolymer, Caprylic/Capric Triglyceride, Ci 77891/Titanium Dioxide, Peg-100 Stearate, Caffeine, Ascorbyl Glucoside, C13-14 Isoparaffin, Cetearyl Glucoside, Dimethiconol, Tocopherol, Sodium Hyaluronate, Retinyl Palmitate, Disodium Edta, Sodium Hydroxide, Parfum/Fragrance, Laureth-7, Adenosine, Myristic Acid, Peg-30 Dipolyhydroxystearate, Sorbitan Oleate, Tin Oxide, Benzyl Salicylate, Benzyl Alcohol, Coumarin, Linalool, Ceteareth-6.

### Production Example 140. Triple Power Anti-Aging Cream, Fragrance Free

Ingredients

Aqua/Water/Eau, Glycerin, Isohexadecane, Isopropyl Isostearate, Niacinamide, Aluminum Starch Octenylsuccinate, Nylon-12, Dimethicone, Cetyl Alcohol, Polyethylene, Stearyl Alcohol, Cetearyl Alcohol, Stearic Acid, Behenyl Alcohol, Phenoxyethanol, Synthetic Fluorphlogopite, Palmitic Acid, Polyacrylamide, Capryloyl Salicylic Acid, Sodium Acrylates Copolymer, Caprylic/Capric Triglyceride, Ci 77891/Titanium Dioxide, Peg-100 Stearate, Caffeine, Ascorbyl Glucoside, C13-14 Isoparaffin, Cetearyl Glucoside, Dimethiconol, Tocopherol, Sodium Hyaluronate, Retinyl Palmitate, Disodium Edta, Sodium Hydroxide, Parfum/Fragrance, Laureth-7, Adenosine, Myristic Acid, Peg-30 Dipolyhydroxystearate, Sorbitan Oleate, Tin Oxide, Benzyl Salicylate, Benzyl Alcohol, Coumarin, Linalool, Ceteareth-6.

### Production Example 141. Daily Re-Volumizing Moisturizer

Ingredients

Aqua/Water, Glycerin, Dimethicone, Isohexadecane, Silica, Hydroxyethylpiperazine Ethane Sulfonic Acid, Alcohol Denat., Dipropylene Glycol, Synthetic Wax, Alpinia Galanga Leaf Extract, Peg-10 Dimethicone, Secale Cereale Extract/Rye Seed Extract, Dimethicone/Peg-10/15 Crosspolymer, Dimethicone/Polyglycerin-3 Crosspolymer, Sodium Acrylates Copolymer, Sodium Hyaluronate, Adenosine, Nylon-12, Dipotassium Glycyrrhizate, Disodium Edta, Caprylic/Capric Triglyceride, Caprylyl Glycol, Synthetic Fluorphlogopite, Pentylene Glycol, Disteardimonium Hectorite, Ethylhexyl Hydroxystearate, Phenoxyethanol, Ci 77163/Bismuth Oxychloride, Ci 77891/Titanium Dioxide, Linalool, Citronellol, Benzyl Alcohol, Benzyl Benzoate, Benzyl Salicylate, Parfum/Fragrance

### Production Example 142. Bright Reveal Dual Overnight Moisturizer

Ingredients

Aqua/Water, Cyclohexasiloxane, Glycerin, Glycolic Acid Alcohol Denat., Propylene Glycol, Dimethicone, Pentaerythrityl Tetraethylhexanoate, Sodium Hydroxide, Polysilicone-11, Hydroxyethylcellulose, Ptfe, Inulin Lauryl Carbamate, Boron Nitride Phenylethyl, Resorcinol, Ammonium Polyacryloyldimethyl Taurate, Phenoxyethanol, Caffeine, Ci 77891/Titanium Dioxide, Ammonium Acryloyldimethyltaurate/Steareth-8 Methacrylate, Copolymer, Mica, Parfum/Fragrance, Ppg-26-Buteth-26, Chlorphenesin, Bisabolol, Ascorbyl Glucoside, Xanthan Gum, Peg-40, Hydrogenated Castor Oil, Limonene, Pentaerythrityl Tetra-Di-T-Butyl Hydroxyhydrocinnamate, Retinyl Palmitate, Disodium Edta, Silica, T-Butyl Alcohol, Hexyl Cinnamal Farnesol, Linalool, Benzyl Salicylate, Benzyl Alcohol, Tin Oxide, Tocopherol

### Production Example 143. Micro-Sculpting Eyeswirl, Eye Cream

Ingredients Water, Cyclopentasiloxane, Glycerin, Niacinamide*, Pentylene Glycol, Acetyl Glucosamine, Titanium Dioxide, Dimethicone Crosspolymer, Panthenol**, Collagen, Palmitoyl Pentapeptide-4***, Tocopheryl Acetate\hat{ }, Sodium PEG-7 Olive Oil Carboxylate, Peucedanum Graveolens (Dill) Extract, Sodium Hyaluronate, Allantoin, Carbomer, Triethanolamine, Phenoxyethanol, Disodium EDTA, Mica, *Vitamin B3, **Pro-Vitamin B5, ***Amino-Peptide, \hat{ }Vitamin E.

### Production Example 144. Cell Renewal Moisturizer

Ingredients

Aqua/Water, Squalane, Cyclohexasiloxane, Glycerin, Cetyl Alcohol, Glyceryl Stearate, Peg-40 Stearate, Paraffinum Liquidum/Mineral Oil, Myristyl Myristate, Pentaerythrityl Tetraethylhexanoate, Octyldodecanol, Sorbitan Tristearate, Butylene Glycol, Acrylamide/Sodium Acryloyldimethyltaurate Copolymer, Phenoxyethanol, Synthetic Fluorphlogopite, Ci 77891/Titanium Dioxide, Isohexadecane, Capryloyl Salicylic Acid, Acrylonitrile/Methyl Methacrylate/Vinylidene Chloride Copolymer, Mannitol, Methylparaben, Ascorbyl Glucoside, Parfum/Fragrance, Chlorphenesin, Stearyl Alcohol, Myristyl Alcohol, Mica, Polysorbate 80, Paeonia Albiflora Root Extract, Disodium Edta, Ci 77163/Bismuth Oxychloride, Ci 77491/Iron Oxides, Hydrolyzed Cicer Seed Extract, Sorbitan Oleate, Isobutane, Sodium Hydroxide, Iris Florentina Root Extract, Vigna Aconitifolia Seed Extract, Cyclodextrin, Sodium Cocoyl Glutamate, Faex Extract/Yeast Extract, Hexyl Cinnamal, Tin Oxide, Benzyl Alcohol, Hydroxycitronellal, Benzyl Salicylate, Linalool, Citronellol, Limonene, Geraniol, Alpha-Isomethyl Ionone, Disodium Succinate

Production Examples 145-147 illustrate active ingredients (external cosmetic agents) suitable for beautification. The external cosmetic agents are suitable for formulation with: (1) suitable inactive ingredients (excipients), and (2) a cannabinoid, terpene, flavonoid, or combination thereof, to provide various topical dosage forms (e.g., creams, gels, lotions, etc.)

### Production Example 145. External Cosmetic Agents

Retinol (vitamin A), green tea extract, acai oil, alpha-lipoic acid, Coenzyme Q10, caffeine, glycolic acid, lactic acid, citric acid, tartaric acid, salicylic acid, L-ascorbic acid, hyaluronic acid, grape seed extract, niacinamide, hydroquinone, jojoba oil, kojic acid, malic acid, resveratrol, vitamin E (tocopherol, tocopheryl acetate), vitamin K, ceramides, raspberry seed oil, plantain extract, collagen, Centella Asiatica, acetyl hexapeptide-3, acetyl hexapeptide-8, beta glucan, carrot root extract, edelweiss extract, ginger root extract, Ginkgo biloba leaf extract, Goji berry extract, Helichrysum extract, polygonum aviculare extract, Vaccinium Vitis Idaea (lingonberry) fruit extract, orange callus culture extract, ferulic acid, tomato extract, teprenone, watermelon extract, turmeric, curcumin, dimethylaminoethanol, elastin, vitamin B5, magnesium oil, colloidal oatmeal, cocoa butter, squalane oil, argan oil, cupuacu butter, avocado oil, Hawaiian kukui nut oil, white tea extract, goat milk, glycerin, allantoin, lanolin, and dimethicone.

### Production Example 146. External Cosmetic Agents

Acetone, alcohol, ammonium alum, potassium alum, aluminum acetate aluminum chlorhydroxy complex, aluminum sulfate, aromatics, benzalkonium chloride, benzethonium chloride, benzocaine, benzoic acid, boric acid, calcium acetate, camphor gum, clove oil, colloidal oatmeal, cresol, cupric sulfate, eucalyptus oil, eugenol, ferric subsulfate (Monsel's solution), honey, isopropyl alcohol, menthol, methyl salicylate, oxyquinoline sulfate, para-tertiary-Butyl-m-cresol, peppermint oil, phenol, polyoxeythylene laurate, potassium ferrocyanide, sage oil, silver nitrate, sodium borate, sodium diacetate, talc, tannic acid glycerite, thymol, topical starch, witch hazel, zinc chloride, zinc oxide, zinc phenolsulfonate, zinc stearate, zinc sulfate, aldioxa, aluminum acetate, aluminum hydroxide, microporous cellulose, cholecalciferol, cocoa butter, cod liver oil, colloidal oatmeal, cysteine hydrochloride, dexpanthenol, dimethicone, glycerin, kaolin, lanolin mineral oil, peruvian balsam, peruvian balsam oil, pramoxine hydrochloride, protein hydrolysate, racemethionine, resorcinol, sulfur, talc, tannic acid, topical starch, vitamin A, live yeast cell derivative, zinc acetate, zinc carbonate, bismuth subnitrate, boric acid, cocoa butter, dimethicone, glycerin, pyridoxine hydrochloride, sulfur, tannic acid, topical starch, trolamine, zinc sulfate, alcohol, ethoxylated alkyl alcohol, strong ammonia solution, ammonium hydroxide, benzalkonium chloride, camphor, ergot fluid extract, ferric chloride, menthol, obtundia surgical dressing, peppermint oil, phenol, pyrilamine maleate, sodium borate, trolamine, turpentine oil, zirconium oxide, beeswax, bismuth subnitrate, boric acid, cetyl alcohol, glyceryl stearate, isopropyl palmitate, live yeast cell derivative, shark liver oil, stearyl alcohol, alcohol anion and cation exchange resins buffered, benzethonium chloride, benzocaine, benzyl alcohol, bismuth subnitrate, bithionol, boric acid, camphor, cetalkonium chloride, chloral hydrate, chlorpheniramine maleate, creosote, diperodon hydrochloride, diphenhydramine hydrochloride, eucalyptus oil, ferric chloride, glycerin, hectorite, hydrogen peroxide, impatiens biflora tincture, iron oxide, isopropyl alcohol, lanolin, lead acetate, lidocaine, menthol, merbromin, mercuric chloride, panthenol, parethoxycaine hydrochloride, phenol, phenyltoloxamine dihydrogen citrate, povidone-vinylacetate copolymers, pyrilamine maleate, salicylic acid, simethicone, tannic acid, topical starch, trolamine, turpentine oil, zirconium oxide, zyloxin, beeswax, bismuth subnitrate, boric acid, cetyl alcohol, glyceryl stearate, isopropyl palmitate, live yeast cell derivative, shark liver oil, stearyl alcohol, beeswax, bismuth subnitrate, boric acid, cetyl alcohol, glyceryl stearate, isopropyl palmitate, live yeast cell derivative, shark liver oil, stearyl alcohol, tannic acid, live yeast cell derivative, zinc acetate, alcohol, allantoin, allantoin, aluminum hydroxide gel, bismuth subnitrate, calamine, cocoa butter, colloidal oatmeal, dimethicone, glycerin, hard fat, kaolin, lanolin, mineral oil, petrolatum, white petrolatum, sodium bicarbonate, sulfur, topical starch, zinc acetate, zinc carbonate, or zinc oxide.

### Production Example 147. External Cosmetic Agents

TABLE A

| ACTIVE | AMOUNT | |
|---|---|---|
| Retinol (vitamin A) | 0.01 wt. %-1 wt. %; | |
| | 0.02-0.2 wt. % | |
| Green tea extract | 0.1-2 | wt. % |
| Acai oil | 2-5 | wt. % |
| Alpha-lipoic acid | 5 | wt. % |
| Coenzyme Q10 | 0.3-1 | wt. % |
| Caffeine | .002 | wt. % |
| Glycolic acid | 5-15 | wt. % |
| Lactic acid | 1-20 | wt. % |
| Citric acid | up to 10 | wt. % |
| Tartaric acid | 5 wt. % (not over | |
| | 20 wt. %) | |
| Salicylic acid | 0.4-3 | wt. % |
| L-ascorbic acid | 0.5-10 | wt. % |
| Hyaluronic acid | 0.1-10 | wt. % |
| Grape seed extract | 5-10 | wt. % |
| Niacinamide | 1-6 | wt. % |
| Hydroquinone | 2 | wt. % |
| Jojoba oil | 1-50 | wt. % |
| Kojic acid | 1-3 | wt. % |
| Malic acid | <1 | wt. % |
| Resveratrol | 1 wt. %-10 wt. % | |
| vitamin E (Tocopherol) | 0.5 wt. %-2.5 wt. % | |
| vitamin E (Tocopheryl acetate) | 1-10 | wt. % |
| Vitamin K | 2-5 | wt. % |
| Ceramides | 1-15 | wt. % |
| Raspberry seed oil | 1-100 | wt. % |
| Plantain extract | 5-10 | wt. % |
| Collagen | 0.2-2 | wt. % |
| Centella asiatica (Gotu kola) extract | 5-10 | wt. % |
| Acetyl Hexapeptide-3 | 10 | wt. % |
| Acetyl Hexapeptide-8 | 3-10 | wt. % |
| Beta Glucan | 1-5 | wt. % |
| Carrot root extract | 1-5 | wt. % |
| Edelweiss extract | 3-5 | wt. % |
| Ginger root extract | 0.1-2 | wt. % |
| Ginkgo Biloba Leaf extract | 0.5-3 | wt. % |
| Goji berry extract | 0.5-5 | wt. % |
| Helichrysum extract | 0.1-3 | wt. % |
| Polygonum aviculare (knotgrass) extract | 0.5-2 | wt. % |
| Vaccinium vitis idaea (lingonberry) fruit | 1-2 wt. % | |
| extract | (plant stem cell) | |
| Turmeric | at least 1 | wt. % |
| Curcumin | 200 | mg/pump |
| Dimethylaminoethanol | 3-5 | wt. % |
| Elastin | 1-5 | wt. % |
| Vitamin B5 | 0.5-5 | wt. % |
| Magnesium oil | ~15 | wt. % |
| Colloidal oatmeal | 5-30 | wt. % |
| Cocoa butter | 3-6 | wt. % |
| Squalane oil | 1-10 | wt. % |
| Argan oil | 1-4 | wt. % |
| Cupuacu butter | 2-5 | wt. % |
| Avocado oil | 0.2-1 | wt. % |
| Hawaiian kukui nut oil | 5 wt. % or more | |
| | (over 23 wt. %) | |
| White tea extract | 3-5 | wt. % |
| goat milk | 38 | wt. % |
| Allantoin | 0.2-2 | wt. % |
| Lanolin alcohol | 0.5-15 | wt. % |
| Lanolin wax | 1-25 | wt. % |
| Dimethicone | 1-20 | wt. % |
| Paullinia Cupana Seed Extract | 0.5-3 | wt. % |
| Acai fruit extract | 0.5-5 | wt. % |
| Multi-herb firming (extracts composed of Asiatic | 0.5-3 | wt. % |
| Centella, Coneflower Seaweed and Fenugreek) | | |
| Knotgrass flavonoids | 0.5-2 | wt. % |
| Carrot seed oil & Beta-Carotene | 0.5-5 | wt. % |
| Coenzyme Q10 and Tocopheryl acetate | 1-3 | wt. % |
| Cephalin | 0.1-2 | wt. % |
| Hydroxy stearic acid | 0.1-1 | wt. % |
| Superoxide Dismutase | 0.2-0.5 | wt. % |
| Carob Tree Gum & Casein | 3-8 | wt. % |
| Dihydromyricetin | 3-7 | wt. % |
| Carob Tree Gum & Wheat Gluten | 2-5 | wt. % |
| Carob Tree Gum | 1-5 | wt. % |
| Amino Acid Complex with Soybean Protein | 3-7 | wt. % |
| Porica Cocos Polysaccharides | 3-6 | wt. % |
| Superoxide Dismutase and soy protein | 3-6 | wt. % |
| Synthetic peptide complex | 1-3 | wt. % |
| Tripeptide-5 & Marrubium extract | 1-5 | wt. % |
| Dunaliella salina micro-algae extract | 1-3 | wt. % |
| Scenedesmus Rubescens Micro-Algae extract | 1-3 | wt. % |
| Asparagus officinalis stem extract | 1-3 | wt. % |
| Paeonia Lactiflora extract | 1-3 | wt. % |
| Marine seaweed extract | 2-5 | wt. % |
| Sesame seed extract | 1-3 | wt. % |
| Amino acid complex with yeast extract | 1-5 | wt. % |
| Sericin protein | 3-7 | wt. % |
| Sodium hyaluronate & Glycosaminoglycans | 2-5 | wt. % |
| Antiaging peptide blend | 1 | wt. % |
| White Genepi Extract | 1-3 | wt. % |
| Summer lilac extract | 1-3 | wt. % |
| Masterwort leaf extract | 1-3 | wt. % |
| Elderberry flower extract | 1-3 | wt. % |
| Rhapontic Rhubarb extract | 0.5-1.5 | wt. % |
| Soybean germ, Wheat germ & Scutellaria root | 2-4 | wt. % |
| Extract Blend | | |
| Rosa Hybrid Flower extract | 0.5-5 | wt. % |
| Kali Musli Extract | 3-5 | wt. % |
| Cinnamon extract | 0.5-5 | wt. % |
| Cocoa Extract | 0.5-5 | wt. % |
| Coffee Seed extract | 0.5-5 | wt. % |
| Roselle plant extract | 0.1-4 | wt. % |
| Licorice root extract | 0.1-2 | wt. % |
| Peppermint leaf extract | 0.5-5 | wt. % |
| Hydrolyzed pearl | 0.5-5 | wt. % |
| Soybean germ & wheat germ extract blend | 1-2 | wt. % |
| Seven herb extract blend (Silybum Marianum | 3-7 | wt. % |
| fruits, Alchemilla Vulgaris plant, Equisetum | | |
| Arvense plant, Soybean seeds, Wheat seeds, | | |
| Alfalfa plant, Radish seeds) | | |
| Multi-vitamin comoplex, hydro-alcoholic | 0.5-5 | wt. % |
| Pumpkin fruit extract | 0.5-5 | wt. % |
| Red clover extract | 0.5-5 | wt. % |
| Rosemary Leaf Extract | 0.1-2 | wt. % |
| Sea Minerals Complex (composed of fucus | 0.5-5 | wt. % |
| vesiculosus, algae extract and sea salt) | | |
| Seaweed oil | 0.5-5 | wt. % |
| vitamin F Glyceric ester | 0.5-5 | wt. % |
| Baicalin | 1-5 | wt. % |
| Red Date Seed Extract | 2-4 | wt. % |
| Indian Soapberry extract | 3-7 | wt. % |
| Yeast extract | 0.5-5 | wt. % |
| Multi-mineral complex (zinc, chlorine, | 0.5-5 | wt. % |
| magnesium, potassium, sodium and lysine) | | |
| Marine green algae extract | 1-3 | wt. % |
| Multi-vitamin complex | 0.5-5 | wt. % |
| Strawberry fruit extract | 0.5-5 | wt. % |
| Natural Anti-Cellulite complex | 2-3 | wt. % |
| Micro-algae extract | 1-3 | wt. % |
| Orange blossom flower water | 5-25 | wt. % |
| Zein & Multi-herb extract complex (Ruscus | 0.5-5 | wt. % |
| Aculeatus Roots, Lemon Peels, Paullinia | | |
| Cupana Seeds, and Corn) | | |
| Chili pepper fruit extract & alcohol | 1-3 | wt. % |
| Fisetin & Raspberry Ketone | 2-5 | wt. % |
| Grape Leaf Extract | 0.1-3 | wt. % |
| Royal Jelly extract | 0.5-5 | wt. % |
| Adenosine, Beta-cyclodextrin | 0.01-0.1 | wt. % |
| Saccharomyces-copper ferment | 0.5-5 | wt. % |
| Folic acid | 0.05-0.2 | wt. % |
| Niacin | 0.01 | wt. % |
| Retinyl acetate (vitamin A) | up to 1 | wt. % |

TABLE B

| ACTIVE | AMOUNT | |
|---|---|---|
| salicylic acid | 0.5-2 wt. % or 2-5 wt. % | |
| Dimethicone | 1-30 | wt. % |
| Lanolin | 12.5-50 | wt. % |
| Retinol | 0.15 wt. % (Monograph not final) | |
| Green tea leaf | 2X; 6X; 10X, 12X, 15X, 30X (unapproved | |
| | homeopathic) | |
| Alpha lipoic acid | 12X; 30X; 60X; 200X (unapproved | |
| | homeopathic) | |
| Coenzyme Q10 | 50 mg (dietary supplement) | |
| Caffeine | 0.01 wt. %; 0.05 wt. % (unapproved | |
| | homeopathic) | |
| Glycolyic acid | 50 mg/mL (approved with 30 mg/mL | |
| | Salicylic acid); 10 wt. % (Unapproved drug | |
| | other) | |
| Hydroquinone | up to 2 | wt. % |

| | |
|---|---|
| Turmeric | 1X, 25 g out of 100 g (unapproved |
| | homeopathic) |
| Colloidal oatmeal | 0.007 wt. % minimum, and a 0.003 wt. % |
| | minimum in combination with mineral oil |
| Cocao butter | 50 wt. % to 100 wt. % |

### Enumerated Embodiments

Specific enumerated embodiments <1> to <104> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

<1> A topical cosmetic composition including the formulation of any one of Production Examples 1-144, further including a cannabinoid, terpene, flavonoid, or combination thereof.

<2> A topical cosmetic composition including the external cosmetic agent of any one of Production Examples 145-147, further including a cannabinoid, terpene, flavonoid, or combination thereof.

<3> A topical cosmetic composition including the formulation of any one of Production Examples 1-146, further including a cannabinoid obtained as a distillate from cannabis.

<4> A topical cosmetic composition including the formulation of any one of Production Examples 1-146, further including a cannabinoid obtained as an extract from cannabis.

<5> A topical cosmetic composition including the formulation of any one of Production Examples 1-146, further including a cannabinoid obtained as a resin from cannabis.

<6> A topical cosmetic composition including the formulation of any one of Production Examples 1-146, further including a cannabinoid isolate obtained from cannabis.

<7> A topical cosmetic composition including the formulation of any one of Production Examples 1-146, further including a cannabinoid obtained from Cannabis indica, Cannabis ruderalis, or Cannabis sativa.

<8> A topical cosmetic composition including the formulation of any one of Production Examples 1-146, further including a cannabinoid present as an oil from cannabis.

<9> A topical cosmetic composition including the formulation of any one of Production Examples 1-146, further including a cannabinoid present as hempseed oil.

<10> A topical cosmetic composition including the formulation of any one of Production Examples 1-146, further including a cannabinoid that is synthetically prepared.

<11> A topical cosmetic composition including the formulation of any one of Production Examples 1-146, further including a cannabinoid that is at least one of THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol) CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), and CBT (cannabicitran).

<12> A topical cosmetic composition including the formulation of any one of Production Examples 1-146, further including a cannabinoid that is at least one of CBD and THC.

<13> A topical cosmetic composition including the formulation of any one of Production Examples 1-146, further including a terpene.

<14> A topical cosmetic composition including the formulation of any one of Production Examples 1-146, further including a terpene that is a sesquiterpene.

<15> A topical cosmetic composition including the formulation of any one of Production Examples 1-146, further including a terpene obtained as a distillate from plant matter.

<16> A topical cosmetic composition including the formulation of any one of Production Examples 1-146, further including a terpene obtained as an extract from plant matter.

<17> A topical cosmetic composition including the formulation of any one of Production Examples 1-146, further including a terpene obtained as a resin from plant matter.

<18> A topical cosmetic composition including the formulation of any one of Production Examples 1-146, further including a terpene obtained from Cannabis sativa, Syzygium aromaticum (cloves), rosemary, or hops.

<19> A topical cosmetic composition including the formulation of any one of Production Examples 1-146, further including a terpene that is synthetically prepared.

<20> A topical cosmetic composition including the formulation of any one of Production Examples 1-146, further including a terpene that is Beta-Caryophyllene.

<21> The topical cosmetic composition of any one of the above embodiments, wherein the amount of external cosmetic agent expressed in terms of concentration (weight percent), present in the formulation of the any one of Production Examples 1-146, remains unchanged with the addition of the cannabinoid, terpene, flavonoid, or combination thereof. For Example, if the amount of external cosmetic agent present in the formulation of the any one of Production Examples 1-146 is 1 wt. % retinol. With the addition of the cannabinoid, terpene, flavonoid, or combination thereof, the resulting amount of the retinol will remain at 1 wt. %.

<22> The topical cosmetic composition of any one of the above embodiments, wherein the amount of inactive ingredient expressed in terms of concentration (weight percent), present in the formulation of the any one of Production Examples 1-146, decreases with the amount added of the cannabinoid, terpene, flavonoid, or combination thereof. For Example, if the amount of external cosmetic agent present in the formulation of the any one of Production Examples 1-146 is 1 wt. % retinol, with the addition of the cannabinoid, terpene, flavonoid, or combination thereof, the resulting amount of the retinol will be less than 1 wt. %.

<23> The topical cosmetic composition of any one of the above embodiments, wherein the aggregate amount of inactive ingredients, present in the formulation of the any one of Production Examples 1-146, decreases in proportion to the amount added of the cannabinoid, terpene, flavonoid, or combination thereof. For Example, if the aggregate amount of inactive ingredients present in the formulation of the any one of Production Examples 1-146 is 90 wt. %. With the addition of 1 wt. % of cannabinoid, terpene, flavonoid, or combination thereof, the resulting aggregate amount of those inactive ingredients will be 89 wt. %.

<24> The topical cosmetic composition of any one of the above embodiments, wherein the aggregate amount of active ingredient (external cosmetic agent) and inactive ingredient in terms of concentration (weight percent), present in the formulation of the any one of Production Examples 1-146, decreases in proportion to the amount added of the cannabinoid, terpene, flavonoid, or combination thereof. For Example, the aggregate amount of active and inactive ingredients present in the formulation of the any one of Production Examples 1-146 is 100 wt. %. With the addition of 1 wt. % of cannabinoid, terpene, flavonoid, or combination thereof, the resulting aggregate amount of those active and inactive ingredients will be 99 wt. %.

<25> The topical cosmetic composition of any one of the above embodiments, wherein the cannabinoid, terpene, flavonoid, or combination thereof, is present in an aggregate amount of up to 20 wt. %.

<26> The topical cosmetic composition of any one of the above embodiments, wherein the cannabinoid, terpene, flavonoid, or combination thereof, is present in an aggregate amount of up to 10 wt. %.

<27> The topical cosmetic composition of any one of the above embodiments, wherein the cannabinoid, terpene, flavonoid, or combination thereof, is present in an aggregate amount of up to 5 wt. %.

<28> The topical cosmetic composition of any one of the above embodiments, wherein the cannabinoid, terpene, flavonoid, or combination thereof, is present in an aggregate amount of up to 1 wt. %.

<29> The topical cosmetic composition of any one of the above embodiments, wherein the cannabinoid, terpene, flavonoid, or combination thereof, is present in an aggregate amount of up to 0.5 wt. %.

<30> The topical cosmetic composition of any one of the above embodiments, wherein the cannabinoid, terpene, flavonoid, or combination thereof, is present in an aggregate amount of 0.330±0.1 wt. %.

<31> The topical cosmetic composition of any one of the above embodiments, wherein the cannabinoid, terpene, flavonoid, or combination thereof, is present in an aggregate amount of 0.1 to 20 wt. %.

<32> The topical cosmetic composition of any one of the above embodiments, wherein the cannabinoid, terpene, flavonoid, or combination thereof, is present in an aggregate amount of 0.1 to 10 wt. %.

<33> The topical cosmetic composition of any one of the above embodiments, wherein the cannabinoid, terpene, flavonoid, or combination thereof, is present in an aggregate amount of 0.1 to 5 wt. %.

<34> The topical cosmetic composition of any one of the above embodiments, wherein the cannabinoid, terpene, flavonoid, or combination thereof, is present in an aggregate amount of 0.01 to 2.5 wt. %.

<35> The topical cosmetic composition of any one of the above embodiments, wherein the cannabinoid, terpene, flavonoid, or combination thereof, is present in an aggregate amount of 0.01 to 1 wt. %.

<36> The topical cosmetic composition of any one of the above embodiments, wherein the cannabinoid, terpene, flavonoid, or combination thereof, is present in an aggregate amount of 0.01 to 0.5 wt. %.

<37> The topical cosmetic composition of any one of the above embodiments, wherein the cannabinoid, terpene, flavonoid, or combination thereof, is present in an aggregate amount of 1 to 20 wt. %.

<38> The topical cosmetic composition of any one of the above embodiments, wherein the cannabinoid, terpene, flavonoid, or combination thereof, is present in an aggregate amount of 1 to 10 wt. %.

<39> The topical cosmetic composition of any one of the above embodiments, wherein the cannabinoid, terpene, flavonoid, or combination thereof, is present in an aggregate amount of 1 to 5 wt. %.

<40> The topical cosmetic composition of any one of the above embodiments, wherein the cannabinoid, terpene, flavonoid, or combination thereof, is present in an aggregate amount of 0.05 to 2.5 wt. %.

<41> The topical cosmetic composition of any one of the above embodiments, wherein the cannabinoid, terpene, flavonoid, or combination thereof, is present in an aggregate amount of 0.05 to 1 wt. %.

<42> The topical cosmetic composition of any one of the above embodiments, wherein the cannabinoid, terpene, flavonoid, or combination thereof, is present in an aggregate amount of 0.05 to 0.5 wt. %.

<43> The topical cosmetic composition of any one of the above embodiments, which is in the form of a gel, pump gel, gel packet, cream, lotion, roll-on liquid, roll-on gel, spray, pump spray, aerosol spray, stick, patch, ointment, liniment, or balm.

<44> A topical cosmetic composition including the formulation of any one of Production Examples 1-146, further including a cannabinoid, terpene, flavonoid, or combination thereof; wherein the cannabinoid, terpene, flavonoid, or combination thereof, while present in the aggregate amounts, are characterized as inactive ingredients.

<45> A topical cosmetic composition including the formulation of any one of Production Examples 1-146, further including a cannabinoid, terpene, flavonoid, or combination thereof; wherein the cannabinoid, terpene, flavonoid, or combination thereof, while present in the aggregate amounts, are characterized as an excipient.

<46> A topical cosmetic composition including the formulation of any one of Production Examples 1-146, further including a cannabinoid, terpene, flavonoid, or combination thereof; wherein the cannabinoid, terpene, flavonoid, or combination thereof, while present in the aggregate amounts, are characterized as active ingredients.

<47> A topical cosmetic composition including the formulation of any one of Production Examples 1-146, further including a cannabinoid, terpene, flavonoid, or combination thereof; wherein the cannabinoid, terpene, flavonoid, or combination thereof, while present in the aggregate amounts, are characterized as an external cosmetic agent.

<48> A topical cosmetic composition including one or more external cosmetic agents of any one of Production Example 145-147, further including a cannabinoid, terpene, flavonoid, or combination thereof, and a carrier system.

<49> A topical cosmetic composition including one or more external cosmetic agents of any one of Production Example 145-147, further including a cannabinoid, and a carrier system.

<50> A topical cosmetic composition including one or more external cosmetic agents of any one of Production Example 145-147, further including a cannabinoid obtained as a distillate from cannabis, and a carrier system.

<51> A topical cosmetic composition including one or more external cosmetic agents of any one of Production Example 145-147, further including a cannabinoid obtained as an extract from cannabis, and a carrier system.

<52> A topical cosmetic composition including one or more external cosmetic agents of any one of Production Example 145-147, further including a cannabinoid obtained as a resin from cannabis, and a carrier system.

<53> A topical cosmetic composition including one or more external cosmetic agents of any one of Production Example 145-147, further including a cannabinoid isolate obtained from cannabis, and a carrier system.

<54> A topical cosmetic composition including one or more external cosmetic agents of any one of Production Example 145-147, further including a cannabinoid obtained from Cannabis indica, Cannabis ruderalis, or Cannabis sativa, and a carrier system.

<55> A topical cosmetic composition including one or more external cosmetic agents of any one of Production Example 145-147, further including a cannabinoid present as an oil from cannabis, and a carrier system.

<56> A topical cosmetic composition including one or more external cosmetic agents of any one of Production Example 145-147, further including a cannabinoid present as hempseed oil, and a carrier system.

<57> A topical cosmetic composition including one or more external cosmetic agents of any one of Production Example 145-147, further including a cannabinoid that is synthetically prepared, and a carrier system.

<58> A topical cosmetic composition including one or more external cosmetic agents of any one of Production Example 145-147, further including a cannabinoid that is at least one of THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol) CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), and CBT (cannabicitran), and a carrier system.

<59> A topical cosmetic composition including one or more external cosmetic agents of any one of Production Example 145-147, further including a cannabinoid that is at least one of CBD and THC, and a carrier system.

<60> A topical cosmetic composition including one or more external cosmetic agents of any one of Production Example 145-147, further including a terpene, and a carrier system.

<61> A topical cosmetic composition including one or more external cosmetic agents of any one of Production Example 145-147, further including a terpene that is a sesquiterpene, and a carrier system.

<62> A topical cosmetic composition including one or more external cosmetic agents of any one of Production Example 145-147, further including a terpene obtained as a distillate from plant matter, and a carrier system.

<63> A topical cosmetic composition including one or more external cosmetic agents of any one of Production Example 145-147, further including a terpene obtained as an extract from plant matter, and a carrier system.

<64> A topical cosmetic composition including one or more external cosmetic agents of any one of Production Example 145-147, further including a terpene obtained as a resin from plant matter, and a carrier system.

<65> A topical cosmetic composition including one or more external cosmetic agents of any one of Production Example 145-1474, further including a terpene obtained from Cannabis sativa, Syzygium aromaticum (cloves), rosemary, or hops, and a carrier system.

<66> A topical cosmetic composition including one or more external cosmetic agents of any one of Production Example 145-147, further including a terpene that is synthetically prepared, and a carrier system.

<67> A topical cosmetic composition including one or more external cosmetic agents of any one of Production Example 145-147, further including a terpene that is Beta-Caryophyllene, and a carrier system.

<68> The topical cosmetic composition of any one of the above embodiments, wherein the cannabinoid, terpene, flavonoid, or combination thereof, is present in an aggregate amount of up to 20 wt. %.

<69> The topical cosmetic composition of any one of the above embodiments, wherein the cannabinoid, terpene, flavonoid, or combination thereof, is present in an aggregate amount of up to 10 wt. %.

<70> The topical cosmetic composition of any one of the above embodiments, wherein the cannabinoid, terpene, flavonoid, or combination thereof, is present in an aggregate amount of up to 5 wt. %.

<71> The topical cosmetic composition of any one of the above embodiments, wherein the cannabinoid, terpene, flavonoid, or combination thereof, is present in an aggregate amount of up to 1 wt. %.

<72> The topical cosmetic composition of any one of the above embodiments, wherein the cannabinoid, terpene, flavonoid, or combination thereof, is present in an aggregate amount of up to 0.5 wt. %.

<73> The topical cosmetic composition of any one of the above embodiments, wherein the cannabinoid, terpene, flavonoid, or combination thereof, is present in an aggregate amount of 0.330±0.1 wt. %.

<74> The topical cosmetic composition of any one of the above embodiments, wherein the cannabinoid, terpene, flavonoid, or combination thereof, is present in an aggregate amount of 0.1 to 20 wt. %.

<75> The topical cosmetic composition of any one of the above embodiments, wherein the cannabinoid, terpene, flavonoid, or combination thereof, is present in an aggregate amount of 0.1 to 10 wt. %.

<76> The topical cosmetic composition of any one of the above embodiments, wherein the cannabinoid, terpene, flavonoid, or combination thereof, is present in an aggregate amount of 0.1 to 5 wt. %.

<77> The topical cosmetic composition of any one of the above embodiments, wherein the cannabinoid, terpene, flavonoid, or combination thereof, is present in an aggregate amount of 0.01 to 2.5 wt. %.

<78> The topical cosmetic composition of any one of the above embodiments, wherein the cannabinoid, terpene, flavonoid, or combination thereof, is present in an aggregate amount of 0.01 to 1 wt. %.

<79> The topical cosmetic composition of any one of the above embodiments, wherein the cannabinoid, terpene, flavonoid, or combination thereof, is present in an aggregate amount of 0.01 to 0.5 wt. %.

<80> The topical cosmetic composition of any one of the above embodiments, wherein the cannabinoid, terpene, flavonoid, or combination thereof, is present in an aggregate amount of 1 to 20 wt. %.

<81> The topical cosmetic composition of any one of the above embodiments, wherein the cannabinoid, terpene, flavonoid, or combination thereof, is present in an aggregate amount of 1 to 10 wt. %.

<82> The topical cosmetic composition of any one of the above embodiments, wherein the cannabinoid, terpene, flavonoid, or combination thereof, is present in an aggregate amount of 1 to 5 wt. %.

<83> The topical cosmetic composition of any one of the above embodiments, wherein the cannabinoid, terpene, flavonoid, or combination thereof, is present in an aggregate amount of 0.05 to 2.5 wt. %.

<84> The topical cosmetic composition of any one of the above embodiments, wherein the cannabinoid, terpene, flavonoid, or combination thereof, is present in an aggregate amount of 0.05 to 1 wt. %.

<85> The topical cosmetic composition of any one of the above embodiments, wherein the cannabinoid, terpene, flavonoid, or combination thereof, is present in an aggregate amount of 0.05 to 0.5 wt. %.

<86> The topical cosmetic composition of any one of the above embodiments, which is in the form of a gel, pump gel, gel packet, cream, lotion, roll-on liquid, roll-on gel, spray, pump spray, aerosol spray, stick, patch, ointment, liniment, or balm.

<87> A topical cosmetic composition including one or more external cosmetic agents of any one of Production Example 145-147, further including a cannabinoid, terpene, flavonoid, or combination thereof, and a carrier system; wherein the cannabinoid, terpene, flavonoid, or combination thereof, while present in the aggregate amounts, are characterized as inactive ingredients.

<88> A topical cosmetic composition including one or more external cosmetic agents of any one of Production Example 145-147, further including a cannabinoid, terpene, flavonoid, or combination thereof, and a carrier system; wherein the cannabinoid, terpene, flavonoid, or combination thereof, while present in the aggregate amounts, are characterized as an excipient.

<89> A topical cosmetic composition including one or more external cosmetic agents of any one of Production Example 145-147, further including a cannabinoid, terpene, flavonoid, or combination thereof, and a carrier system; wherein the cannabinoid, terpene, flavonoid, or combination thereof, while present in the aggregate amounts, are characterized as active ingredients.

<90> A topical cosmetic composition including one or more external cosmetic agents of any one of Production Example 145-1474, further including a cannabinoid, terpene, flavonoid, or combination thereof, and a carrier system; wherein the cannabinoid, terpene, flavonoid, or combination thereof, while present in the aggregate amounts, are characterized as an external cosmetic agent.

<91> The topical cosmetic composition of any one of the above embodiments, which is in the form of a cream, gel, lotion, patch, liniment, spray, roll-on, stick, or balm.

<92> The topical cosmetic composition of any one of embodiments <1> to <91>, having a total THC content of less than 0.05 mg/mL, wherein the total THC content is defined as the amount of THCA/mL*0.877, plus the amount of THC/mL*1, expressed as:

Total THC content=(amount of THCA/mL*0.877)+(amount of THC/mL*1)<0.05 mg/mL

<93> The topical cosmetic composition of any one of embodiments <1> to <92>, having a total THC content of less than 0.025 mg/mL, wherein the total THC content is defined as the amount of THCA/mL*0.877, plus the amount of THC/mL*1, expressed as:

Total THC content=(amount of THCA/mL*0.877)+(amount of THC/mL*1)<0.025 mg/mL

<94> The topical cosmetic composition of any one of embodiments <1> to <93>, substantially free from (a)-(e):

(a) Tetrahydrocannabinol (THC),

(b) Tetrahydrocannabinolic Acid (THCA),

(c) Cannabidiolic Acid (CBDA),

(d) Cannabinol (CBN),

(e) Cannabigerol (CBG),

such that any of (a)-(e) present in the topical cosmetic composition is present such that the topical cosmetic composition includes each in no more than 0.01 mg/mL.

<95> The topical cosmetic composition of any one of embodiments <1> to <94>, substantially free from (a)-(e):

(a) Tetrahydrocannabinol (THC),

(b) Tetrahydrocannabinolic Acid (THCA),

(c) Cannabidiolic Acid (CBDA),

(d) Cannabinol (CBN),

(e) Cannabigerol (CBG),

such any one or more of (a)-(e) that is present in the topical cosmetic composition is present in a total amount of up to 0.02 mg/mL.

<96> A method that includes topically administering to a subject the topical cosmetic composition of any one of the above embodiments.

<97> The method of embodiment <96>, wherein the topical cosmetic composition is administered to the skin surface of the subject to treat or prevent at least one of: wrinkles, fine lines, blemishes, skin discoloration, dry skin, skin irritation, saggy skin, inelastic skin, enlarged pores, acne scars, blackheads, whiteheads, inflammation, crow's feet, laugh lines, drooping eyelids, crepey skin, frown lines, dull skin tone, dark circles under the eyes, lackluster skin, itchy skin, hyperpigmentation, uneven skin tone, dry skin, itchy skin, and collagen loss.

<98> The method of any one of embodiments <96> to <97>, wherein the topical cosmetic composition is applied to a clean and dry topical skin surface of the subject.

<99> The method of any one of embodiments <96> to <98>, wherein the topical cosmetic composition is applied to at least one of the face and neck of the subject.

<100> The method of any one of embodiments <96> to <99>, wherein the topical cosmetic composition is applied up to four times a day.

<101> The method of any one of embodiments <96> to <100>, wherein the subject is at least 12 years old.

<102> The method of any one of embodiments <96> to <100>, wherein the subject is at least 18 years old.

<103> The method of any one of embodiments <96> to <102>, wherein the subject is a human.

<104> The method of any one of embodiments <96> to <103>, wherein after administering to a topical skin surface, the hands are washed with soap and water.

1. A method comprising topically administering to a skin surface of a human subject a topical cosmetic composition comprising:
external cosmetic agent;
solvent;
emulsifier;
thickening agent;
emollient; and
at least one of cannabinoid, terpene, and flavonoid;
wherein,
the topical cosmetic composition is substantially free from (a)-(c): (a) Cannabidiolic Acid (CBDA), (b) Cannabinol (CBN), and (c) Cannabigerol (CBG); such that any of (a)-(c) present in the topical cosmetic composition is present such that the topical cosmetic composition includes each in no more than 0.01 mg/mL; and
the topical cosmetic composition has a total THC content of less than 0.02 mg/mL, wherein the total THC content is defined as the amount of THCA*0.877, plus the amount of THC*1, expressed as:
Total THC content=(amount of THCA/mL*0.877)+(amount of THC/mL*1)<0.02 mg/mL.

2. The method of claim 1, wherein the topical cosmetic composition is administered to the skin surface of the subject to treat or prevent at least one of: wrinkles, fine lines, blemishes, skin discoloration, dry skin, skin irritation, saggy skin, inelastic skin, enlarged pores, acne scars, inflammation, crow's feet, laugh lines, drooping eyelids, crepey skin, frown lines, dull skin tone, dark circles under the eyes, lackluster skin, itchy skin, hyperpigmentation, uneven skin tone, and collagen loss.

3. The method of claim 1, wherein the external cosmetic agent comprises at least one of retinol (vitamin A), green tea extract, acai oil, alpha-lipoic acid, Coenzyme Q10, caffeine, glycolic acid, lactic acid, citric acid, tartaric acid, salicylic acid, L-ascorbic acid, hyaluronic acid, grape seed extract, niacinamide, hydroquinone, jojoba oil, kojic acid, malic acid, resveratrol, vitamin E (tocopherol, tocopheryl acetate), vitamin K, ceramides, raspberry seed oil, plantain extract, collagen, Centella Asiatica, acetyl hexapeptide-3, acetyl hexapeptide-8, beta glucan, carrot root extract, edelweiss extract, ginger root extract, Ginkgo biloba leaf extract, Goji berry extract, Helichrysum extract, polygonum aviculare extract, Vaccinium Vitis Idaea (lingonberry) fruit extract, orange callus culture extract, ferulic acid, tomato extract, teprenone, watermelon extract, turmeric, curcumin, dimethylaminoethanol, elastin, vitamin B5, magnesium oil, colloidal oatmeal, cocoa butter, squalane oil, argan oil, cupuacu butter, avocado oil, Hawaiian kukui nut oil, white tea extract, goat milk, glycerin, allantoin, lanolin and dimethicone.

4. The method of claim 1, wherein the external cosmetic agent is present in 0.1-7.5 wt. %.

5. The method of claim 1, wherein the solvent comprises at least one of water, glycerin, propylene glycol, propanediol, butylene glycol, ethoxydiglycol, isododecane, isohexadecane, octyldodecanol, hexylene glycol, 1,2-hexane diol, and dicaprylyl carbonate.

6. The method of claim 1, wherein the solvent is present in 60-80 wt. % of the topical cosmetic composition.

7. The method of claim 1, wherein the emulsifier comprises at least one of polysorabate 60, laureth-4, potassium cetyl sulfate, cetyl alcohol, cetearyl alcohol, stearyl alcohol, glyceryl stearate, propylene glycol, polyglyceryl-6 laurate, ceteareth-20, PEG-100 stearate, sodium lauroyl lactylate, myristyl myristate, carbomer, polysorbate 80, polawax, sorbitan stearate, gum Arabic, brassica alcohol, carbomer 980 QD, sodium stearate, polyhydroxystearic acid, PEG-150 distearate, glyceryl oleate, emulsifying wax, glyceryl monooleate, phospholipids, polyoxyethylene alkyl ethers, polyoxyethylene, castor oil derivatives, sorbitan fatty acid esters, polyoxyethylene stearates, polyoxylglycerides, polysorbate, and sorbitan esters.

8. The method of claim 1, wherein the emulsifier is present in 3-8 wt. % of the topical cosmetic composition.

9. The method of claim 1, wherein the thickening agent comprises at least one of Carbopol 980, carbomer, cetyl alcohol, stearic acid, carnauba wax, hydroxyethyl cellulose, guar gum, xanthan gum, gelatin, magnesium aluminum silicate, silica, bentonite, cetyl palmitate, ammonium acryloyldimethyltaurate, cetearyl alcohol, glucose-D, hectorite gel, stearyl palmitate, gum arabic, hydroxypropyl starch phosphate, tapioca starch, acrylates octylacrylamide copolymer, carbomer 940, polyamide 3, castor wax, hydroxypropyl methylcellulose, caesalpinia spinosa gum, brassica alcohol, carbomer 980 QD, sodium stearate, polyhydroxystearic acid, tribehenin, arrowroot starch, rice starch, candelilla wax, beeswax, ozokerite wax, sunflower wax, PEG-150 distearate, polyacrylate crosspolymer-6, acrylates C10-30 alkyl acrylate crosspolymer, hydroxypropyl guar, and cyclopentasiloxane (silicone gel).

10. The method of claim 1, wherein the thickening agent is present in 0.3-3 wt. % of the topical cosmetic composition.

11. The method of claim 1, wherein the emollient comprises at least one of beeswax, olive oil, coconut oil, lanolin oil (isolanolin), petrolatum (petroleum jelly), mineral oil, glycerin, methyl glyceth, zinc oxide, goat milk, butyl stearate, diglycol laurate, C12-15 alkyl benzoate, aloe vera palmitate, butylene glycol, ethylhexyl palmitate, isododecane, isohexadecane, isopropyl myristate, octyldodecanol, PEG-40 hydrogenated castor oil, pentylene glycol BIO, (1, 3) propanediol, allantoin, lecithin, caprylic capric triglyceride, octyl deodecanol, cetearyl alcohol, squalene, squalene, tocopheryl acetate, polyglyceryl-6 laurate, ceteareth-20, dimethicone, PEG-100 stearate, octyldodecanol, caprylyl glycol, dicaprylyl carbonate, myristyl myristate, hectorite gel, stearyl palmitate, polyamide 3, sorbitol, polyhydroxystearic acid, lauryl laurate, and cyclopentasiloxane (silicone gel).

12. The method of claim 1, wherein the emollient is present in 10-22 wt. % of the topical cosmetic composition.

13. The method of claim 1, wherein the topical cosmetic composition is formulated as a cream or lotion.

14. The method of claim 1, wherein the external cosmetic agent is dispersed throughout the topical composition.

15. The method of claim 1, wherein the external cosmetic agent is at least partially dissolved in the solvent.

16. The method of claim 1, wherein the topical cosmetic composition further comprises at least one of a pH adjusting agent, antioxidant, buffer, transdermal delivery agent, preservative, fragrance, coloring agent, and exfoliant.

17. The method of claim 1, wherein the topical cosmetic composition further comprises a pH adjusting agent comprising at least one of triethanolamine, citric acid, sodium gluconate, magnesium hydroxide, sodium bicarbonate, and lactic acid.

18. The method of claim 1, wherein the topical cosmetic composition further comprises a preservative comprising at least one of butylparaben, ethylparaben, methylparaben, propylparaben, sorbic acid, benzyl alcohol, salicylic acid, formaldehyde, tetrasodium ethylenediaminetetra-acetic acid (EDTA), neroli hydrosol, phenoxyethanol, ethylhexylglycerin, methylisothiazolinone, methylchloroisothiazolinone, citric acid, sodium benzoate, and tetrasodium glutamate diacetate.

19. The method of claim 1, wherein the topical cosmetic composition further comprises a transdermal delivery agent comprising at least one of dimethyl sulfoxide (DMSO), transcutol® (2-(2-ethoxyethoxy) ethanol), ethanol, and isopropyl alcohol.

20. The method of claim 1, wherein the topical cosmetic composition further comprises an antioxidant comprising at least one of tocopherol (vitamin E), tocopheryl acetate (vitamin E acetate), anthocyanins, proanthocyanins, alpha-lipoic acid, catechins, retinol (vitamin A), beta-carotene (vitamin A), Coenzyme Q10, chlorogenic acid, curcumin, vitamin C, theograndin, luteolin, acetophenone derivatives, monoterpenes, sesquiterpenes, triterpenes, phytosterols, ferulic acid, and lycopene.

21. The method of claim 1, wherein the topical cosmetic composition further comprises an antioxidant present in 0.05-0.5 wt. %.

22. The method of claim 1, wherein the topical cosmetic composition further comprises a fragrance comprising at least one of blood orange, citrus punch, clean linen, coconut, coconut lime verbena, coral reef, lemon verbena, mandarin berry, mangosteen, natural rose, neroli, pineapple lily, pink grapefruit and passion fruit, white peach and ginger, white tea, lavender, peppermint, sweet fennel, sweet orange, tea tree, woodland essential, vanilla, cherry blossom, white jasmine, apple blossom, pumpkin, cinnamon, peony, apple, blackberry, cranberry, plum blossom, grapefruit, ginger flower, freesia petal, chamomile, eucalyptus, violet, sweet pea, frangipani, pomegranate, honeydew, cantaloupe, white gardenia, pink nectarine, cotton musk, magnolia blossom, sage, cedarwood, freesia, black currant, white patchouli, sweet almond, and cashmere musk.

23. The method of claim 1, wherein the topical cosmetic composition further comprises a coloring agent comprising at least one of caramel colorant, Red No.6 D&C Lake liquid, Red No.7 D&C Lake liquid, Blue No.1 FD&C Lake, Red No.28 D&C Lake, Red No.40 FD&C Lake, Red No.7 D&C Lake, Light Red No.7 D&C Lake, Violet No.2 External D&C, Yellow No.5 FD&C Lake, mica beige, mica blackstar red, mica Bordeaux, mica bronze, mica carmine red, mica cinnamon, mica diamond cluster, mica diamond sparkle, mica fine silver, mica gold, mica gold rush, mica interference copper, mica interference green, mica interference violet, mica light blue, mica luster black, mica magenta, mica majectic green, mica patina silver, mica pearl white, mica pink camel, mica purple sky, mica red, mica sand gold, mica sappan red, mica sugar blush, and mica walnut brown.

24. The method of claim 1, wherein the topical cosmetic composition further comprises an exfoliant comprising at least one of glycolic acid, lactic acid, citric acid, tartaric acid, salicylic acid, avocado powder, bacillus ferment, bamboo stem powder, pumpkin puree, glucosamine HCl, Laminario digitata extract, Saccharomyces cerevisiae extract, urea, walnut shell powder, and yogurt filtrate.

25. The method of claim 1, wherein the topical cosmetic composition further comprises an exfoliant present in 1-2 wt. %.
